(19) United States
(12) Patent Application Publication
(10) Pub. No.: US 2005/0261836 A1
(43) Pub. Date: Nov. 24, 2005

(54) Crystal structure of mitogen-activated protein kinase-activated protein kinase 2 and binding pockets thereof

(76) Inventors: Wuyi Meng, Westborough, MA (US); Lovorka Swenson, Belmont, MA (US)

Correspondence Address: FISH & NEAVE IP GROUP;ROPES & GRAY LLP, 1251 AVENUE OF THE AMERICAS FL C3, NEW YORK, NY, 10020-1105, US

(21) Appl. No.: 10/497,767

(22) PCT Filed: Dec. 5, 2002
(86) PCT No.: PCT/US02/39070

§ 371 Date: Nov. 23, 2004

Related U.S. Application Data
(60) Provisional application No. 60/337,513, filed on Dec. 5, 2001.

(51) Int. Cl. G06F 19/00; G01N 33/48; G01N 33/50

(52) U.S. Cl. 702/19

(57) ABSTRACT

The invention relates to crystalline molecules or molecular complexes that comprise binding pockets of mitogen activated protein kinase activated protein kinase 2 (MAPKAPK2) or its homologues. The invention also relates to crystals comprising MAPKAPK2. The present invention also relates to a computer comprising a data storage medium encoded with the structural coordinates of MAPKAPK2 binding pockets and methods of using a computer to evaluate the ability of a compound to bind to the molecule or molecular complex. This invention also relates to methods of using the structure coordinates to solve the structure of homologous proteins or protein complexes. In addition, this invention relates to methods of using the structure coordinates to screen for, design and optimize compounds, including agonists and antagonists, which bind to MAPKAPK2 or homologues thereof.

### TECHNICAL FIELD OF THE INVENTION

The present invention relates to crystalline molecules or molecular complexes that comprise binding pockets of the Mitogen-activated Protein Kinase-activated Protein Kinase 2 (MAPKAPK2) and its homologues, the structure of these molecules or molecular complexes, and methods of using these molecules or molecular complexes.

### BACKGROUND OF THE INVENTION

Protein kinases mediate intracellular signal transduction by affecting a phosphoryl transfer from a nucleoside triphosphate to a protein acceptor involved in a signaling pathway. There are a number of kinases and pathways through which extracellular and other stimuli cause a variety of cellular responses to occur inside the cell. Examples of such stimuli include environmental and chemical stress signals (e.g., osmotic shock, heat shock, ultraviolet radiation, bacterial endotoxin, H₂O₂), cytokines (e.g., interleukin-1 (IL-1) and tumor necrosis factor-α (TNF-α)), growth factors (e.g., granulocyte macrophage-colony-stimulating factor (GM-CSF), and fibroblast growth factor (FGF). An extracellular stimulus may effect one or more cellular responses related to cell growth, migration, differentiation, secretion of hormones, activation of transcription factors, muscle contraction, glucose metabolism, control of protein synthesis and regulation of cell cycle. Many disease states are associated with abnormal cellular responses triggered by protein kinase-mediated events. These diseases include autoimmune diseases, inflammatory diseases, neurological and neurodegenerative diseases, cancer, cardiovascular diseases, allergies and asthma, Alzheimer's disease and hormone-related diseases. Thus, an understanding of the structure, function, and inhibition of kinase activity could lead to useful human therapeutics.

Among medically important kinases are the serine/threonine kinases, which include the mammalian mitogen-activated protein (MAP) kinases. MAP kinases are activated by dual phosphorylation of threonine and tyrosine at the Thr-X-Tyr segment in the activation loop. Members of the MAP kinase family also share sequence similarity and conserved structural domains, and include the extracellular-signal regulated kinases (ERKs), Jun N-terminal kinases (JNKs) and p38 kinases. MAP kinases also phosphorylate various substrates including transcription factors, which in turn regulate the expression of specific sets of genes and mediate a specific response to the stimulus.

For instance, in response to cellular stresses, such as heat or osmotic shock [Rouse et al., Cell, pp. 1027-037 (1994)], bacterial lipopolysaccharide [Han et al., Science, 265, pp. 808-811 (1994)], proinflammatory cytokines and TNF-α [Freshney et al., Cell, 78, pp. 1039-1049 (1994)], a subfamily of MAP kinase, p38/RK (reactivating kinase), are activated by upstream kinases, including MEK3 (MKK3), MEK6 (MKK6) [Han et al., J. Biol. Chem., 271, pp. 2886-2891 (1996); Lin et al., Science, 268, pp. 286-290 (1995); Raingeaud et al., Mol. Cell. Biol., 16, pp. 1247-1255 (1996)] and SEK1 (MKK4 or JNKK1, [Sanchez et al., Nature, 372, pp. 794-798 (1994)]. Upon activation, p38 phosphorylates MAPKAPK2 [Stokoe et al., EMBO. J., 11, pp. 3985-3994 (1992)], MAPKAPK3/3pk [McLaughlin et al., J. Biol. Chem., 271, pp. 8488-8492 (1996)], PRAK (p38-related/activated protein kinase, [New et al., EMBO. J., 17, pp. 3372-3384 (1998)], MNK1/2 (MAP kinase interacting kinase, [Waskiewicz et al., EMBO. J., 16, pp. 1909-1920 (1997); Fukunaga et al., EMBO. J., 16, pp. 1921-1933 (1997)], MSK1 (mitogen and stress activated kinase, [Deak et al., EMBO. J., 17, pp. 4426-4441 (1998)] and transcription factors ATF2 (activating transcription factor, [Jiang et al., J. Biol. Chem., 271, pp. 17920-17926 (1996)], CHOP/GADD153 [Wang et al., Mol. Cell. Biol., 16, pp. 4273-4280 (1996)], Elk-1 [Whitmarsh et al., Mol. Cell. Biol., 17, pp. 2360-2371 (1997)], SAP1a [Janknecht et al., Oncogene, 10, pp. 1209-1216 (1995)] and MEF2C (myocyte enhancer factor, [Han et al., Nature, 386, pp. 296-299 (1997)]. The MAP kinases also mediate intracellular signal transduction pathways [M. H. Cobb et al., J. Biol. Chem., 270, pp. 14843-6 (1995); R. J. Davis, Mol. Reprod. Dev., 42, pp. 459-67 (1995)]; cell proliferation and apoptosis [M. J. Robinson and M. H. Cobb, Curr. Opin. Cell Biol., 9, pp. 189-186].

Human MAPKAPK2 [Engel et al., FEBS Lett., 336, pp. 143-147 (1993); Stokoe et al., Biochem. J., 296, pp. 843-849 (1993); Zu et al., Biochem. Biophys. Res. Commun., 200, pp. 1118-1124 (1994)], a 400 residue enzyme has two proline-rich segments at the N-terminus followed by the kinase domain and a C-terminal regulatory domain. The N-terminal proline rich segments have been shown to bind to the c-ABL SH3 domain in vitro [Plath et al., Biochem. Biophys. Res. Commun., 203, pp. 1188-1194 (1994)]. The kinase domain has low homology with other serine/threonine kinases except MAPKAPK3/4 (FIG. 3). The N-terminal proline-rich domain and C-terminal regulatory domain share no significant homology with other non-MAPKAP proteins. The C-terminal regulatory domain contains a bipartite nuclear localization signal and a nuclear export signal [Ben-Levy et al., Curr. Biol., 8, pp. 1049-1057 (1998); Engel et al., EMBO. J., 17, pp. 3363-3371 (1998)].

MAPKAPK2 was originally identified as a kinase that is phosphorylated and activated in vitro by p42/p44 (ERK1/ERK2), isoforms of MAP kinases, and inactivated by protein phosphatase 2A (PP2A, [Stokoe et al., FEBS Lett., 313, pp. 307-313 (1992)]. Later studies have shown that MAPKAPK2 is activated in vivo only by p38/p40/RK [Freshney et al., Cell, 78, pp. 1039-1049 (1994); Guay et al., J. Cell. Sci., 110, pp. 357-368 (1997); Han et al., Science, 265, pp. 808-811 (1994); Rouse et al., Cell, 78, pp. 1027-1037 (1994)]. In fact, mice that lack MAPKAPK2 show increased stress resistance and survive bacterial LPS-induced endotoxic shock due to a 90% reduction in the production of tumor necrosis factor-α [Kotlyarov et al., Nat. Cell. Biol., 1, pp. 94-97 (1999)].

MAPKAPK2 is located in the nucleus of unstimulated cells and rapidly moves to the cytoplasm upon stimulation [Ben-Levy et al., Curr. Biol., 8, pp. 1049-1057 (1998)]. While in the nucleus, MAPKAPK2 contributes to the phosphorylation of CREB (cAMP response element-binding protein) at Ser133 and may regulate its ability to activate transcription in response to cAMP, Ca²⁺ and nerve growth factors [Ginty et al., Cell, 77, pp. 713-725 (1994); Gonzalez and Montminy, Cell, 59, pp. 675-680 (1989); Sheng et al., Science, 252, pp. 1427-1430 (1991)]. MAPKAPK2 also phosphorylates serum response factor at Ser103 both in vivo and in vitro in response to tumor-promoting and stress-inducing stimuli [Heidenreich et al., J. Biol. Chem., 274, pp. 14434-14443 (1999)]. Furthermore, both MAPKAPK2 and MAPKAPK3/3pk (chromosome 3p kinase) interact with basic helix-loop-helix transcription factor E47 in vivo and phosphorylate E47 in vitro, suggesting that they are regulators of E47 activity and E47-dependent gene expression [Neufeld et al., J. Biol. Chem., 275, pp. 20239-20242 (2000)]. In the cytoplasm, MAPKAPK2 phosphorylates the small heat shock protein HSP25/HSP27 [Sutherland et al., Eur. J. Biochem., 217, pp. 715-722 (1993)] and the lymphocyte specific protein (LSP1)[Huang et al., J. Biol. Chem., 272, pp. 17-19 (1997)], which are both F-actin binding proteins. Other substrates of MAPKAPK2 include glycogen synthase [Stokoe et al., EMBO. J., 11, pp. 3985-3994 (1992)], tyrosine hydroxylase, the rate-limiting enzyme in catecholamine synthesis [Stokoe et al., FEBS Lett., 313, pp. 307-313 (1992); Sutherland et al., Eur. J. Biochem., 217, pp. 715-722 (1993)], and 5-lipoxygenase, a key enzyme in leukotriene biosynthesis [Werz et al., Proc. Nat'l. Acad Sci USA, 97, pp. 5261-5266 (2000)].

Accordingly, there has been an interest in finding MAPKAPK2 inhibitors that are effective as therapeutic agents. A challenge has been to find protein kinase inhibitors that act in a selective manner. Since there are numerous protein kinases that are involved in a variety of cellular responses, non-selective inhibitors may lead to unwanted side effects. In this regard, the three-dimensional structure of the kinase would assist in the rational design of inhibitors. The determination of the amino acid residues in MAPKAPK2 binding pockets and the determination of the shape of those binding pockets would allow one to design inhibitors that bind favorably to this class of enzymes.

Furthermore, despite the fact that the genes and the crystal structures for various kinases are known, no one has described X-ray crystal structural coordinate information of any of the MAPKAP kinases. Such information would be extremely useful in identifying and designing potential inhibitors of various MAPKAP kinases, which, in turn, could have therapeutic utility.

### SUMMARY OF THE INVENTION

Applicants have solved this problem by providing, for the first time, a crystallizable composition and crystal comprising MAPKAPK2. The crystal was resolved at 2.8 Å resolution. Solving this crystal structure has allowed applicants to determine the key structural features of MAPKAPK2, particularly the shape of its substrate and ATP-binding pockets, and more particularly the mechanism of its nuclear export with p38.

Thus, the present invention provides molecules or molecular complexes comprising all or part of the MAPKAPK2 binding pockets, or MAPKAPK2-like binding pockets that have similar three-dimensional shapes.

The invention further provides a computer comprising a data storage medium that comprises the structure coordinates of molecules and molecular complexes comprising all or part of the MAPKAPK2 or MAPKAPK2-like binding pockets and means for extracting three-dimensional structural information from the structure coordinates. The computer may be used to produce three-dimensional information of the crystalline molecule or molecular complex comprising such binding pockets.

The invention provides methods for screening, designing, optimizing, evaluating and identifying compounds that bind to the molecules or molecular complexes or their binding pockets. The methods can be used to identify agonists and antagonists of MAPKAPK2 and its homologues.

The invention also provides a method for determining at least a portion of the three-dimensional structure of molecules or molecular complexes which contain at least some structurally similar features to MAPKAPK2, particularly MAPKAPK2 homologues. This is achieved by using at least some of the structural coordinates obtained from the MAPKAPK2 structure.

The invention also provides a method for crystallizing MAPKAPK2, a MAPKAPK2 protein complex, or homologues thereof.

### BRIEF DESCRIPTION OF THE DRAWINGS

FIG. 1 lists the atomic structure coordinates for MAPKAPK2 as derived by X-ray diffraction from the crystal. The following abbreviations are used in FIG. 1:

“Atom type” refers to the element whose coordinates are measured. The first letter in the column defines the element.

“Res” refers to the amino acid residue.

“X, Y, Z” crystallographically define the atomic position of the element measured.

“B” is a thermal factor that measures movement of the atom around its atomic center.

“Occ” is an occupancy factor that refers to the fraction of the molecules in which each atom occupies the position specified by the coordinates. A value of “1” indicates that each atom has the same conformation, i.e., the same position, in all molecules of the crystal.

“Mol” refers to the molecule in the asymmetric unit.

FIG. 2 depicts a ribbon diagram of the overall fold of MAPKAPK2. The N-terminal domain (around residues 44 to 327), the C-terminal domain (around residues 328 to 400) and the regulatory loop are shown. Key features of the kinase, such as the Thr334 and the activation-loop, are indicated. The dotted line within the activation-loop are regions where the electron density was poor and no model was built.

FIG. 3 is a sequence alignment of MAPKAPK2 (indicated as “mk2”, SEQ ID NO: 1), MAPKAPK3 (indicated as “mk3”, SEQ ID NO: 2), calcium/calmodulin-dependent protein kinase I (CaMKI)(SEQ ID NO: 3), and cyclic adenosine monophosphate-dependent protein kinase (cAPK)(SEQ ID NO: 4). The secondary structure of MAPKAPK2 (αB and α2 starting from the N-terminus) and cAPK are shown above the sequences. The secondary structure nomenclature of cAPK follows that used in Knighton et al., Science, 253, pp. 414-420 (1991)]. Residues that are identical and similar in the four sequences are indicated.

FIG. 4 depicts a superposition of molecule A (light shade) with molecule B (dark shade) (FIG. 4A); molecule B (dark shade) with cAPK (light shade) (FIG. 4B); and molecule B (dark shade) with CaMKI (light shade) (FIG. 4C).

FIG. 5 depicts a superposition of MAPKAPK2 (dark shade) with active cAPK (light shade). The catalytically important residues Lys93, Glu104, Arg185, Asp186, Asp207 and Asp366 are labeled.

FIG. 6 depicts the interaction between the kinase domain C-lobe and the regulatory domain second helix.

FIG. 7 depicts the nuclear export signal (NES) structure of MAPKAPK2 and p53 (FIG. 7A) and the sequence alignment of the leucine-rich NES between MAPKAPK2, PKI, p53 and Rev (FIG. 7B).

FIG. 8 shows a surface representation of the MAPKAPK2 substrate binding pocket.

FIG. 9 shows a diagram of a system used to carry out one embodiment of the instructions encoded by the storage medium of FIGS. 10 and 11.

FIG. 10 shows a cross section of a magnetic storage medium.

FIG. 11 shows a cross section of a optically-readable data storage medium.

### DETAILED DESCRIPTION OF THE INVENTION

In order that the invention described herein may be more fully understood, the following detailed description is set forth.

Throughout the specification, the word “comprise”, or variations such as “comprises” or “comprising” will be understood to imply the inclusion of a stated integer or groups of integers but not exclusion of any other integer or groups of integers.

The following abbreviations are used throughout the application:

| | | | | | |
|---|---|---|---|---|---|
| A = | Ala = | Alanine | T = | Thr = | Threonine |
| V = | Val = | Valine | C = | Cys = | Cysteine |
| L = | Leu = | Leucine | Y = | Tyr = | Tyrosine |
| I = | Ile = | Isoleucine | N = | Asn = | Asparagine |
| P = | Pro = | Proline | Q = | Gln = | Glutamine |
| F = | Phe = | Phenylalanine | D = | Asp = | Aspartic Acid |
| W = | Trp = | Tryptophan | E = | Glu = | Glutamic Acid |
| M = | Met = | Methionine | K = | Lys = | Lysine |
| G = | Gly = | Glycine | R = | Arg = | Arginine |
| S = | Ser = | Serine | H = | His = | Histidine |

Additional definitions are set forth below.

The term “about” when used in the context of RMSD values takes into consideration the standard error of the RMSD value, which is ±0.1 Å.

The term “active site” refers to the portion of the protein kinase to which the nucleotides bind. The active site of MAPKAPK2 is at the N-terminus between two proline-rich segments and the C-terminal regulatory domain [See, Meng et al., J. Biol. Chem., 277(40), pp. 37401-37405 (2002), incorporated herein by reference].

The term “ATP analogue” refers to a compound derived from adenosine-5′-triphosphate (ATP). The compound can be adenosine, AMP, ADP, or a non-hydrolyzable analogue, such as, but not limited to AMP-PNP. The analogue may be in complex with magnesium or manganese ions.

The term “associating with” refers to a condition of proximity between a chemical entity or compound, or portions thereof, and a binding pocket or binding site on a protein. The association may be non-covalent—wherein the juxtaposition is energetically favored by hydrogen bonding or van der Waals or electrostatic interactions—or it may be covalent.

The term “binding pocket”, as used herein, refers to a region of a molecule or molecular complex, that, as a result of its shape and/or charge, favorably associates with another chemical entity or compound. The term “pocket” includes, but is not limited to, a cleft, channel or site or a combination thereof. MAPKAPK2 or MAPKAPK2-like molecules may have binding pockets, which include, but are not limited to, peptide or substrate binding sites, and ATP-binding sites.

The term “part of a binding pocket” refers to less than all of the amino acid residues that define the binding pocket. For example, the structure coordinates of residues that constitute part of a binding pocket may be specific for defining the chemical environment of the binding pocket, or useful in designing fragments of an inhibitor that may interact with those residues. For example, the portion of residues may be key residues that are spatially related and define a three-dimensional compartment of the binding pocket. The residues may be contiguous or non-contiguous in primary sequence. In one embodiment, part of a binding pocket has at least two amino acid residues, preferably at least four, six or eight amino acid residues.

The term “chemical entity”, as used herein, refers to chemical compounds, complexes of at least two chemical compounds, and fragments of such compounds or complexes that bind to MAPKAPK2 or a homologue thereof. The chemical entity may be, for example, a ligand, a substrate, a nucleotide triphosphate, a nucleotide diphosphate, phosphate, a nucleotide, an agonist, antagonist, inhibitor, antibody, drug, peptide, protein or compound.

The term “conservative substitutions” refers to residues that are physically or functionally similar to the corresponding reference residues. That is, a conservative substitution and its reference residue have similar size, shape, electric charge, chemical properties including the ability to form covalent or hydrogen bonds, or the like. Preferred conservative substitutions are those fulfilling the criteria defined for an accepted point mutation in Dayhoff et al., Atlas of Protein Sequence and Structure, 5, pp. 345-352 (1978 & Supp.), which is incorporated herein by reference. Examples of conservative substitutions are substitutions including but not limited to the following groups: (a) valine, glycine; (b) glycine, alanine; (c) valine, isoleucine, leucine; (d) aspartic acid, glutamic acid; (e) asparagine, glutamine; (f) serine, threonine; (g) lysine, arginine, methionine; and (h) phenylalanine, tyrosine.

The term “correspond to” or “corresponding amino acid” when used in the context of amino acid residues that correspond to MAPKAPK2 amino acids refers to particular amino acids or analogues thereof in a MAPKAPK2 homologue that corresponds to amino acids in the MAPKAPK2 protein. The corresponding amino acid may be an identical, mutated, chemically modified, conserved, conservatively substituted, functionally equivalent or homologous amino acid when compared to the MAPKAPK2 amino acid to which it corresponds.

Methods for identifying a corresponding amino acid are known in the art and are based upon sequence, structural alignment, its functional position or a combination thereof as compared to the MAPKAPK2 protein. For example, corresponding amino acids may be identified by superimposing the backbone atoms of the amino acids in MAPKAPK2 and the MAPKAPK2 homologue using well known software applications, such as QUANTA (Accelrys, San Diego, Calif. ©2001, 2002). The corresponding amino acids may also be identified using sequence alignment programs such as the “bestfit” program available from the Genetics Computer Group that uses the local homology algorithm described by Smith and Waterman in Advances in Applied Mathematics 2, 482 (1981), which is incorporated herein by reference.

The term “crystallization solution” refers to a solution that promotes crystallization. The solution comprises at least one agent, and may include a buffer, one or more salts, a precipitating agent, one or more detergents, sugars or organic compounds, lanthanide ions, a poly-ionic compound and/or a stabilizer.

The term “domain” refers to a portion of the MAPKAPK2 protein or homologue that can be separated according to its biological function, for example, catalysis or regulatory function. The domain is usually conserved in sequence or structure when compared to other kinases or related proteins. The domain can comprise a binding pocket, or a sequence or structural motif. In MAPKAPK2 protein, the protein is separated into two domains, the N-terminal catalytic domain and the C-terminal regulatory domain.

The term “full length protein” or “full length MAPKAPK2” refers to the complete MAPKAPK2 protein (amino acid residues 1 to 400 of SEQ ID NO: 1), which includes the standard two-lobe kinase architecture plus an extra C-terminal regulatory domain.

The term “generating a three-dimensional structure” refers to converting the lists of structure coordinates into structural models in three-dimensional space. This can be achieved through commercially or publicly available software. The three-dimensional structure may be displayed as a graphical representation or used to perform computer modeling or fitting operations. In addition, the structure coordinates themselves may be used to perform computer modeling and fitting operations.

The term “homologue of MAPKAPK2” or “MAPKAPK2 homologue” refers to a molecule or molecular complex that is homologous to MAPKAPK2 by three-dimensional structure or sequence, but retains the kinase activity of a mitogen-activated protein kinase activated protein kinase. Examples of homologues include but are not limited to the following: human MAPKAPK1, MAPKAPK1-beta, MAPKAPK2, MAPKAPK3, MAPKAPK4, chromosome 3p kinase with mutations, conservative substitutions, additions, deletions or a combination thereof; and MAPKAPK1, MAPKAPK1-beta, MAPKAPK2, MAPKAPK3, MAPKAPK4, chromosome 3p kinase from a species other than human, or with mutations, conservative substitutions, additions, deletions or a combination thereof.

The term “homology model” refers to a structural model derived from known three-dimensional structure(s). Generation of the homology model, termed “homology modeling”, can include sequence alignment, residue replacement, residue conformation adjustment through energy minimization, or a combination thereof.

The “MAPKAPK2 ATP-binding pocket” of a molecule or molecular complex is defined by the structure coordinates of a certain set of amino acid residues present in the MAPKAPK2 structure, as described below. In general, the ligand for the ATP-binding pocket is a nucleotide such as ATP. This binding pocket is in the active site of the kinase domain. In the protein kinase family, the ATP binding pocket is generally located at the interface of the α-helical and β-strand domain, and is bordered by the glycine rich loop and the hinge [See, Xie et al., Structure, 6, pp. 983-991 (1998), incorporated herein by reference].

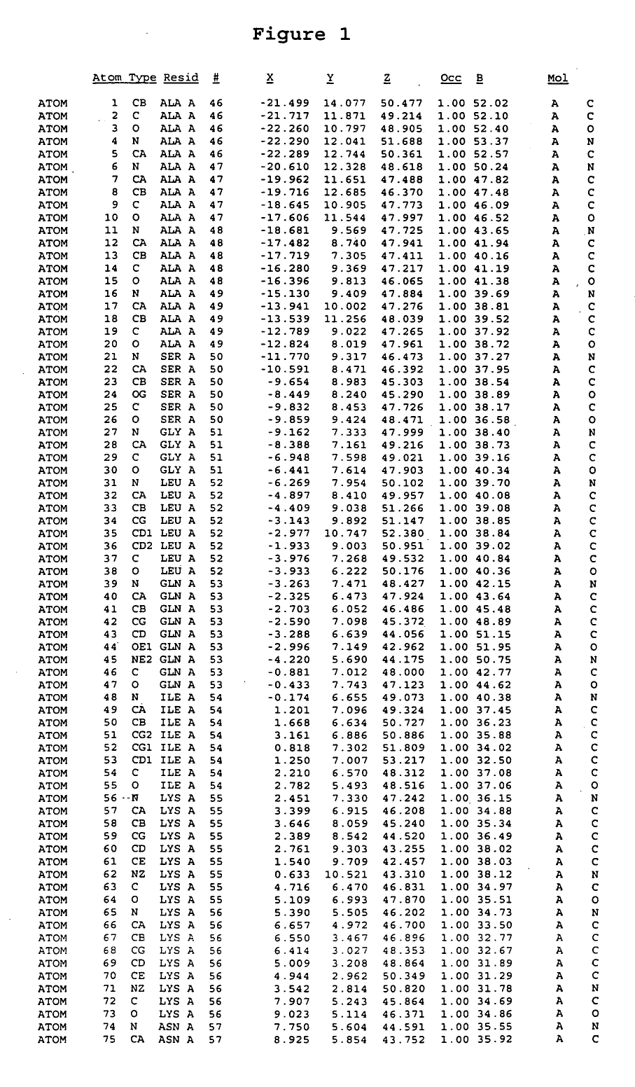

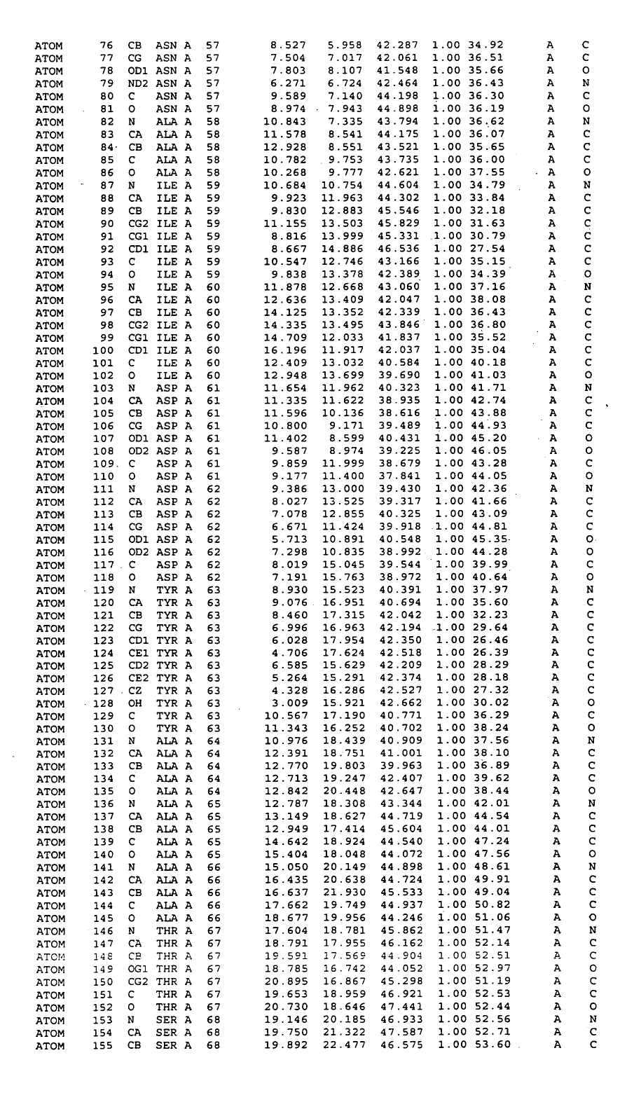

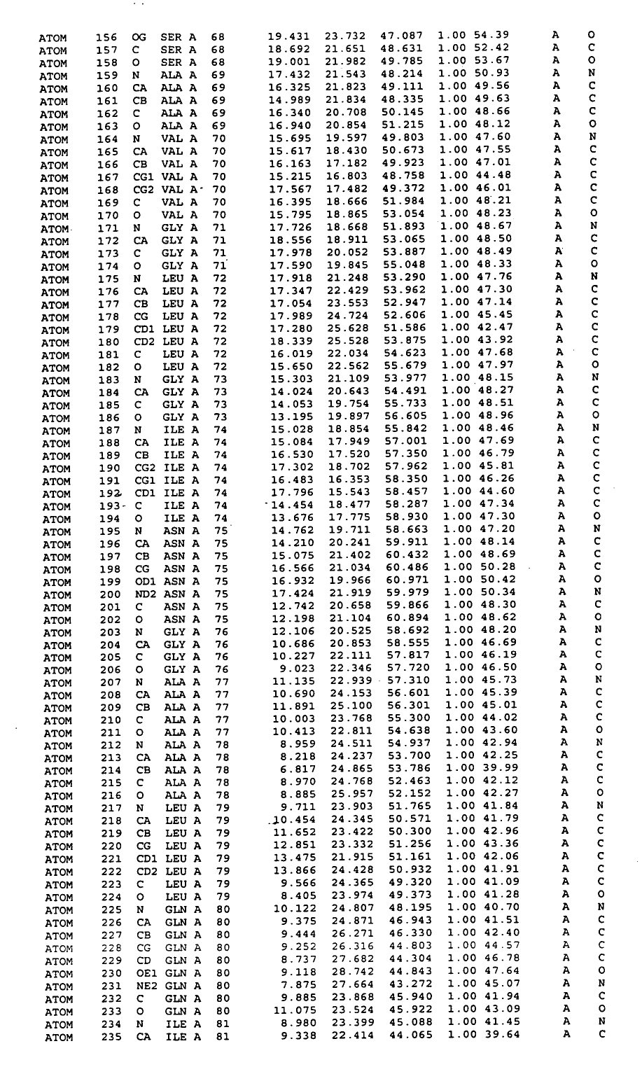

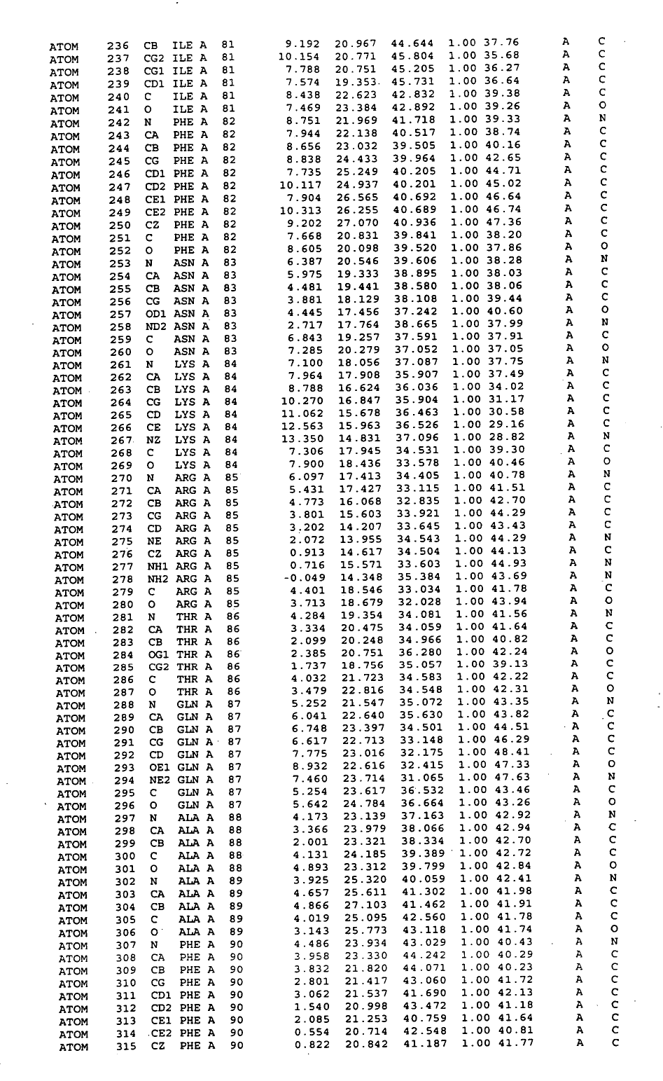

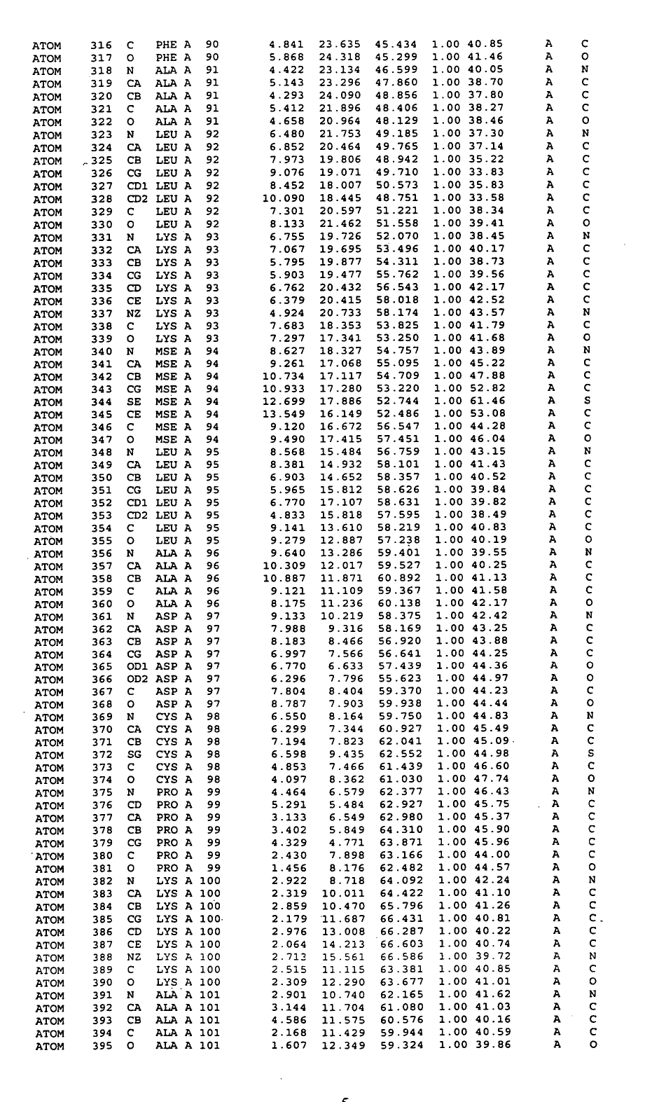

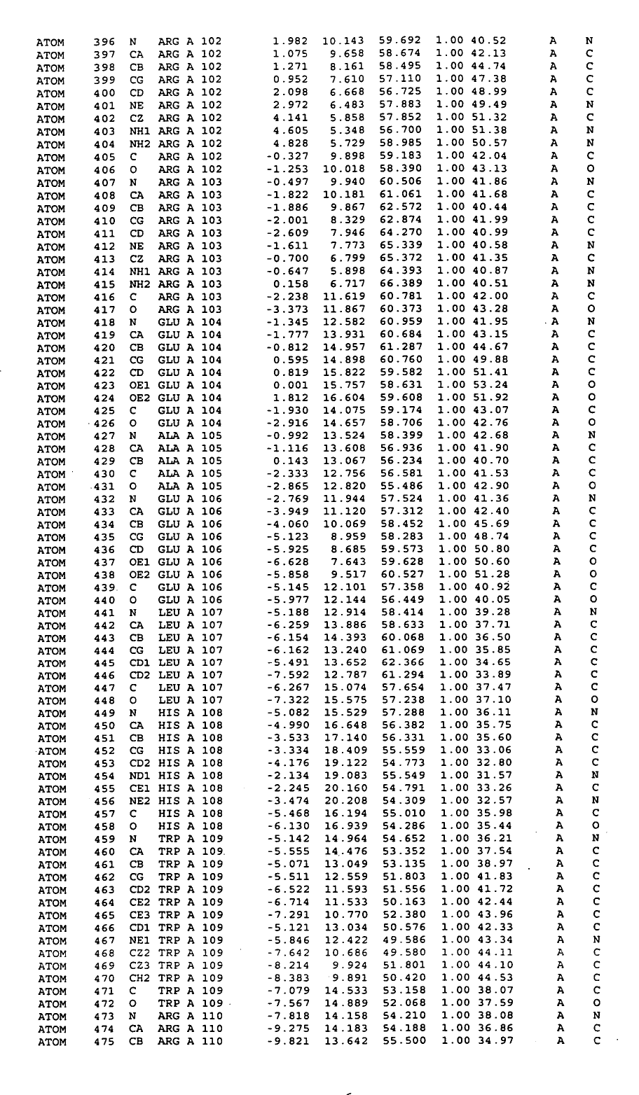

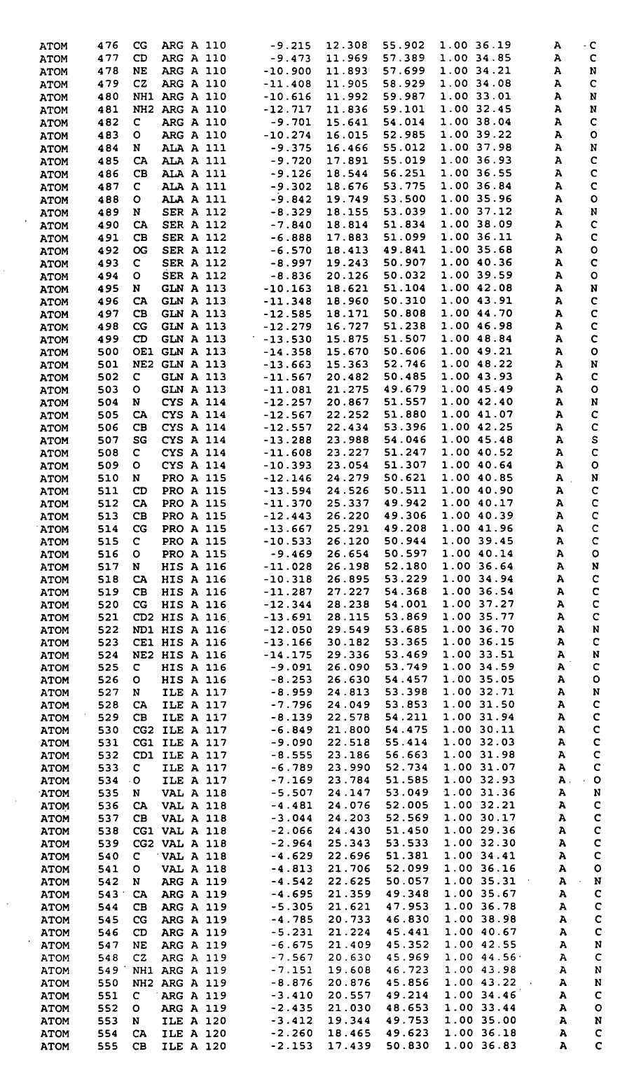

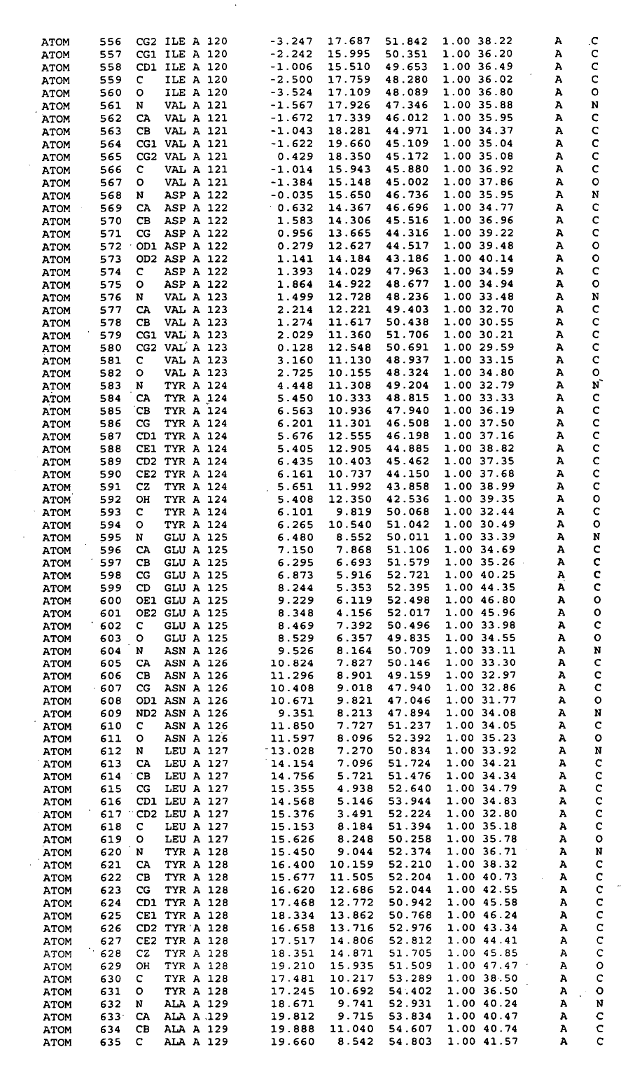

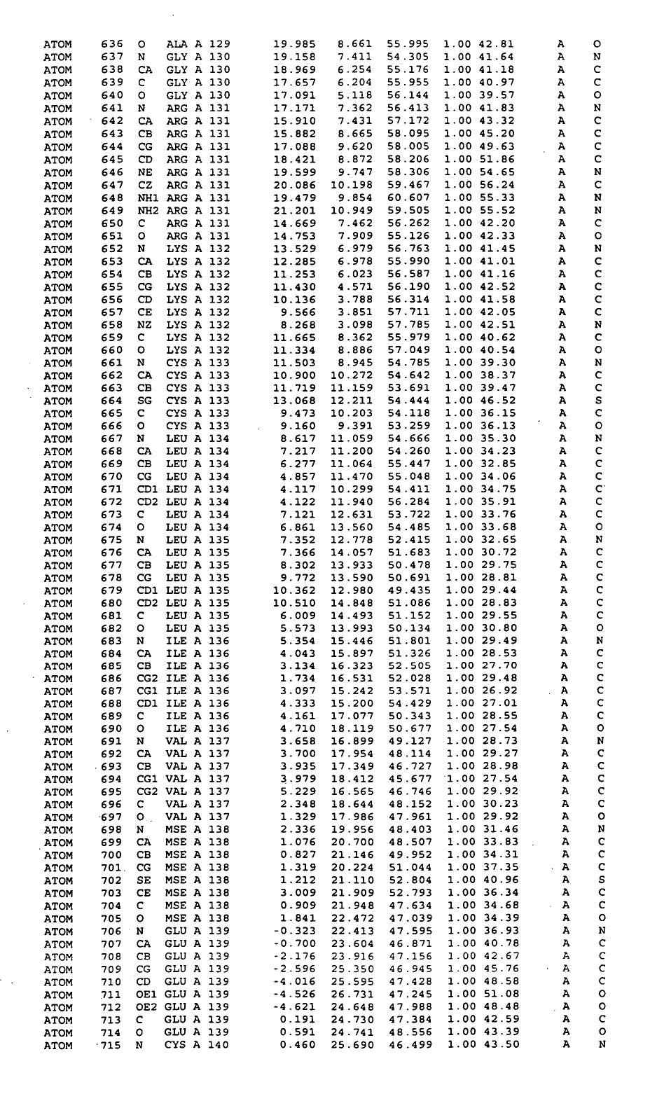

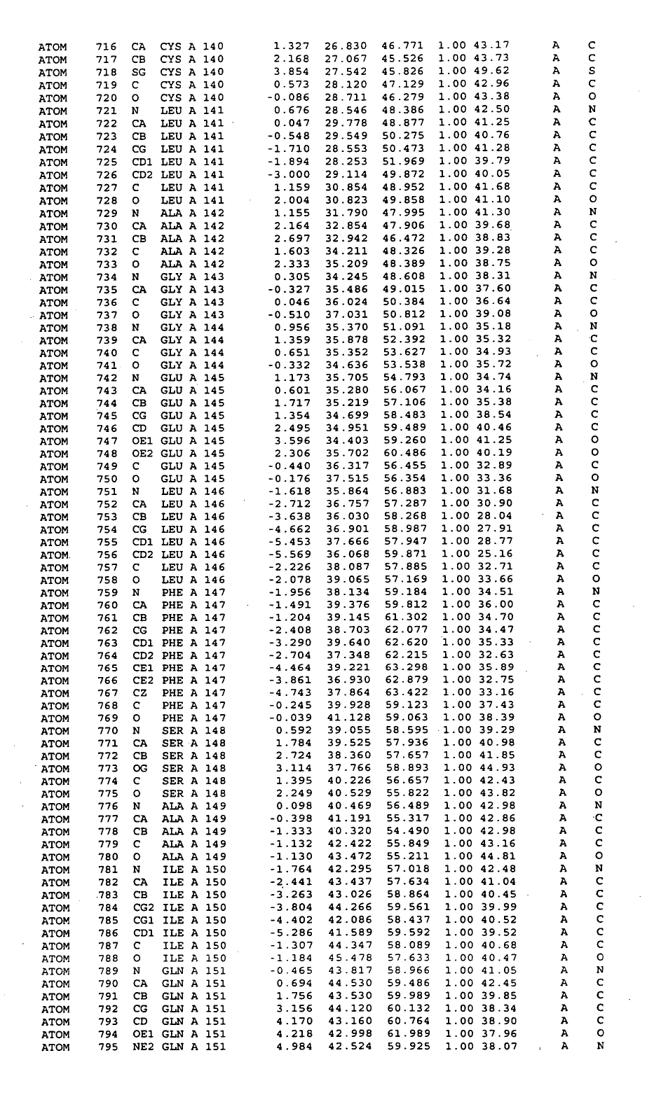

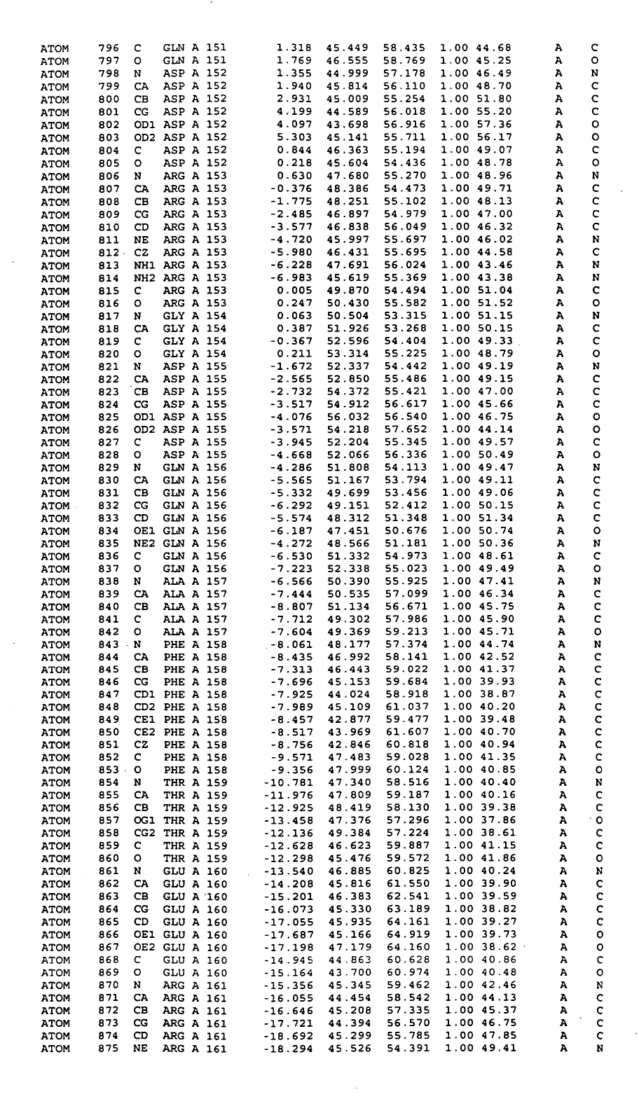

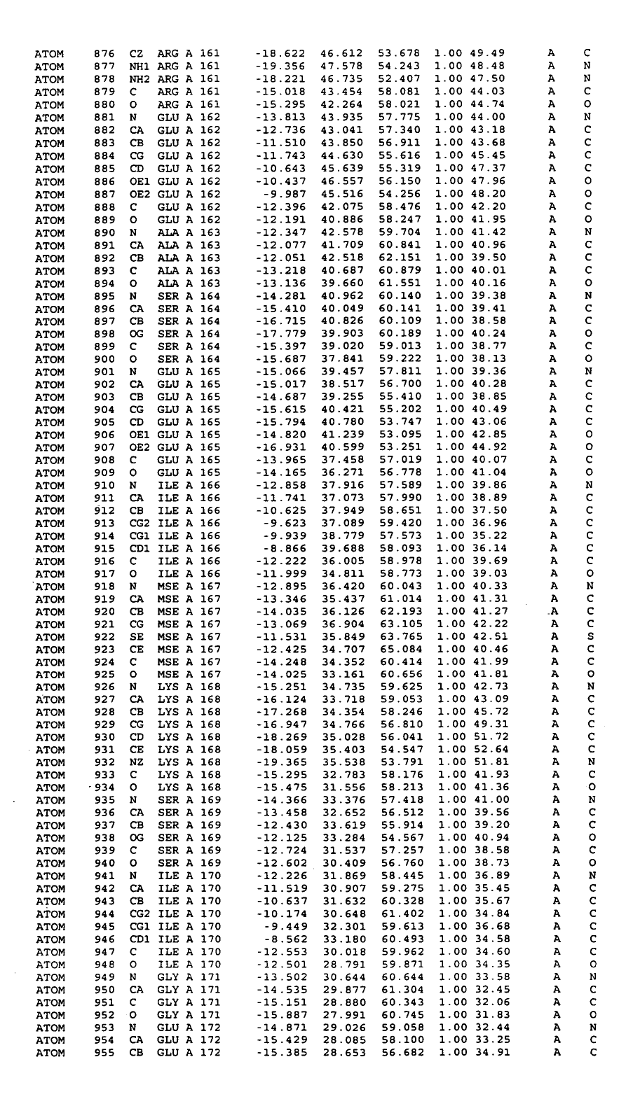

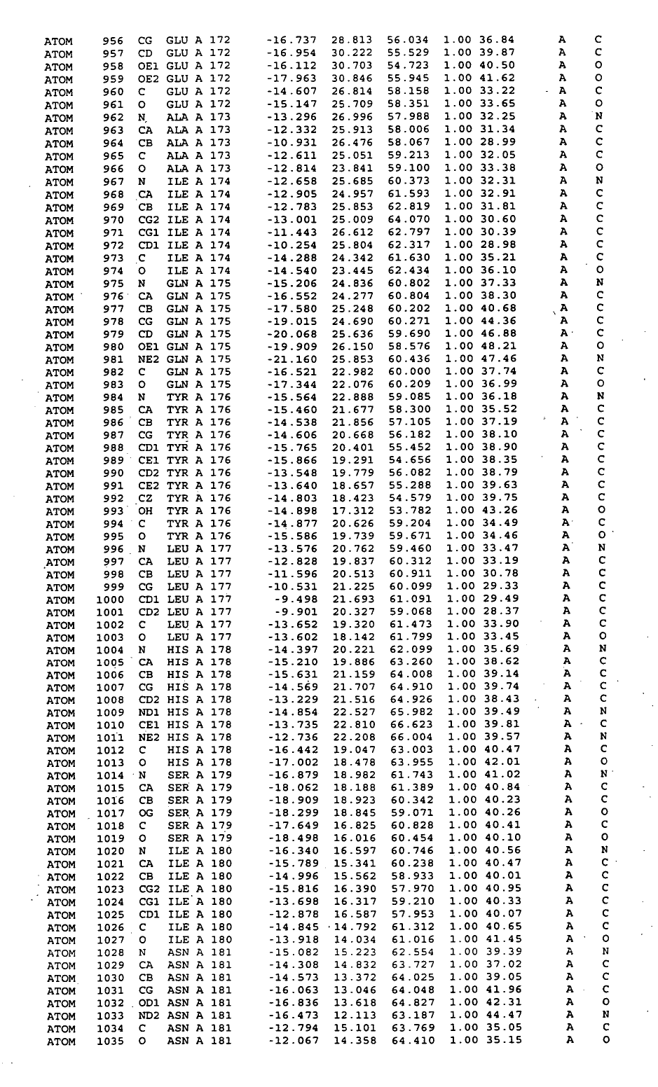

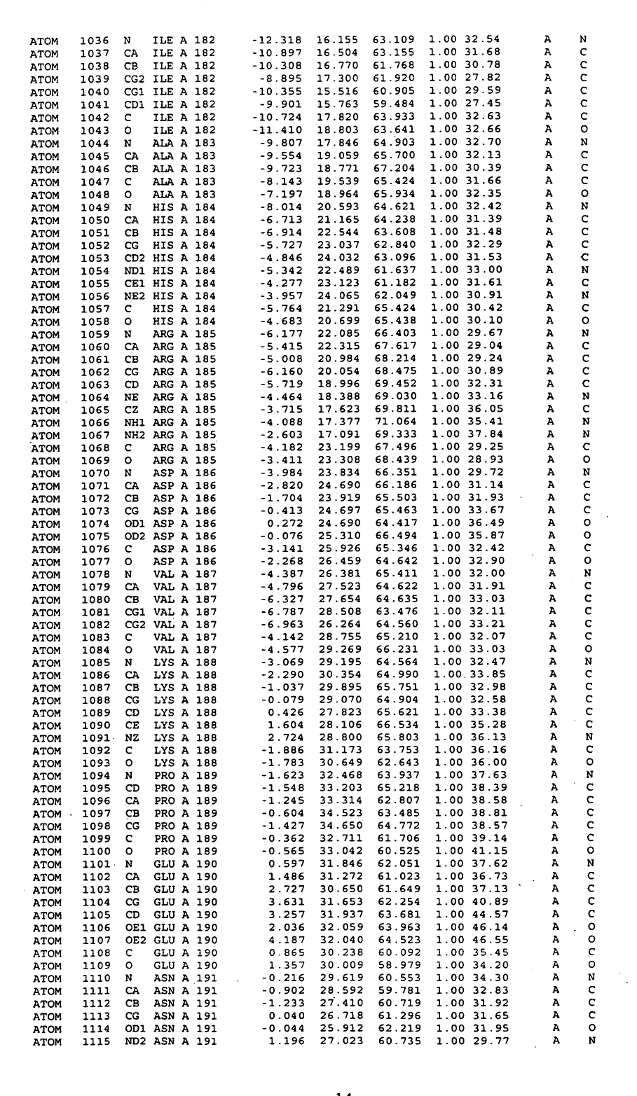

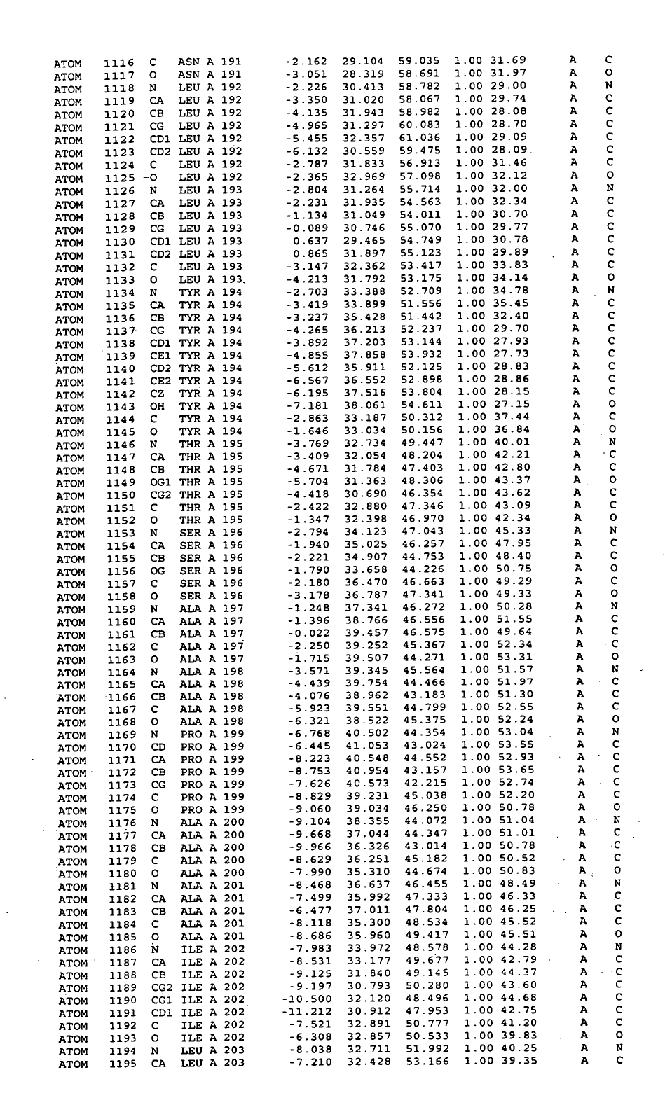

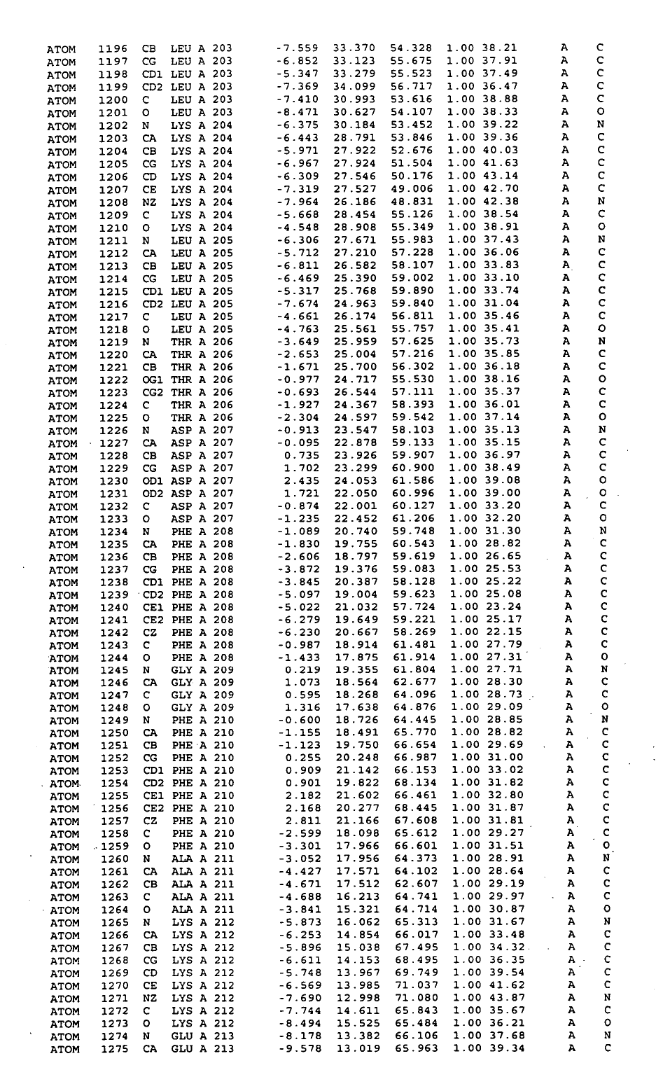

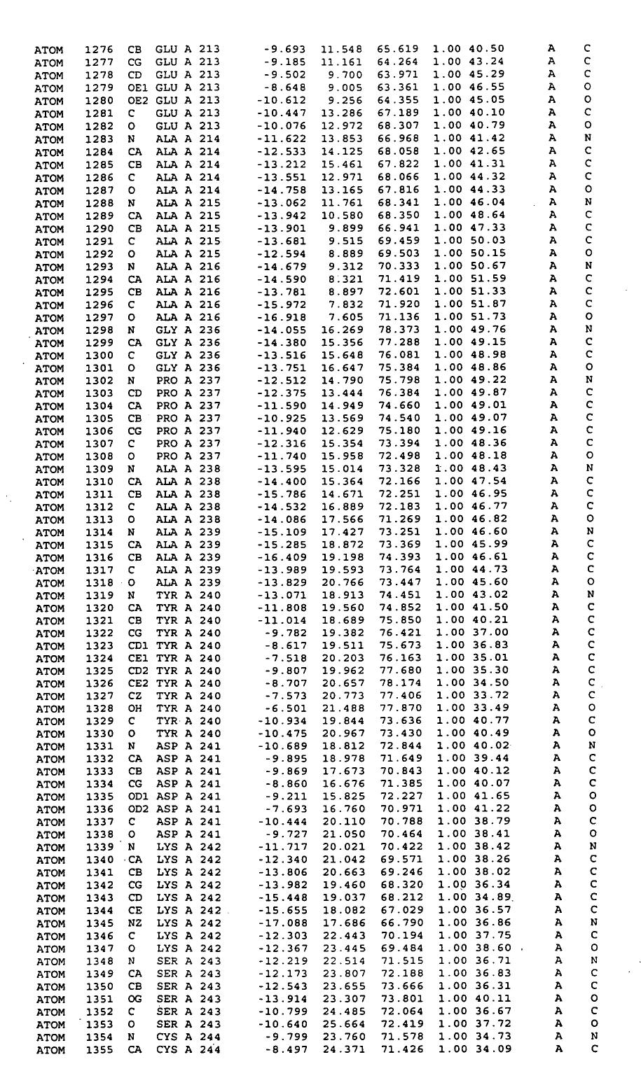

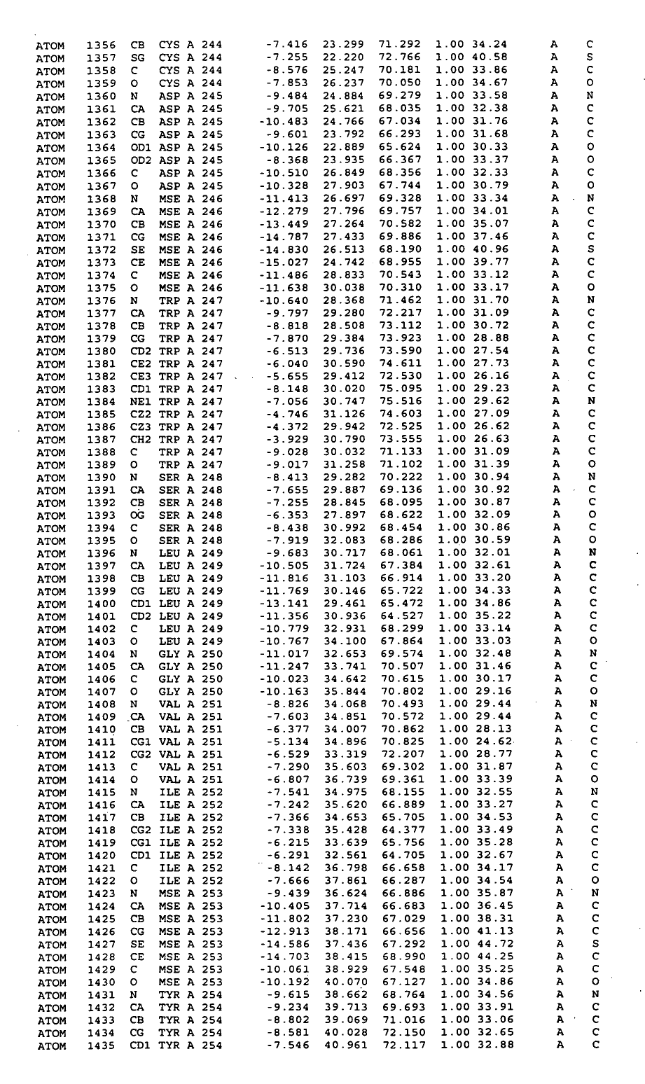

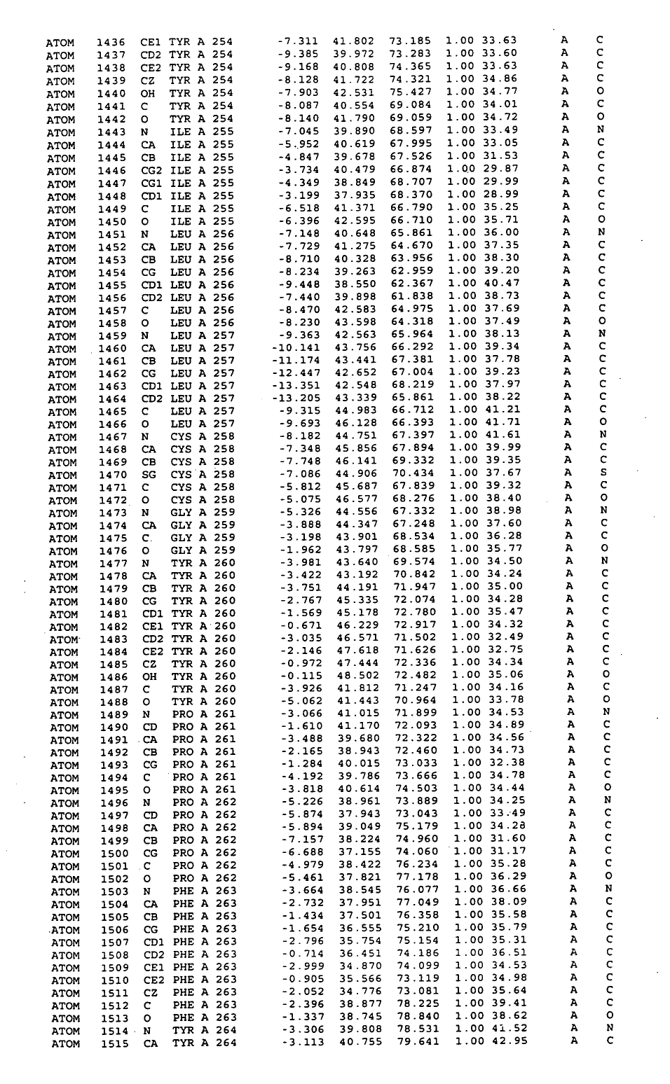

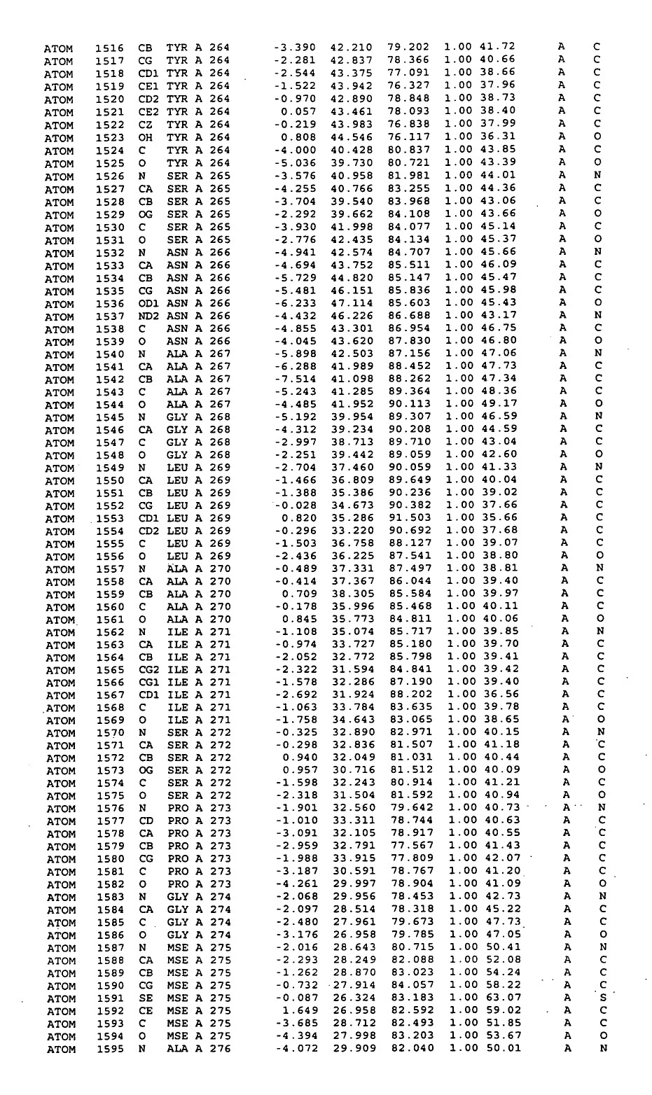

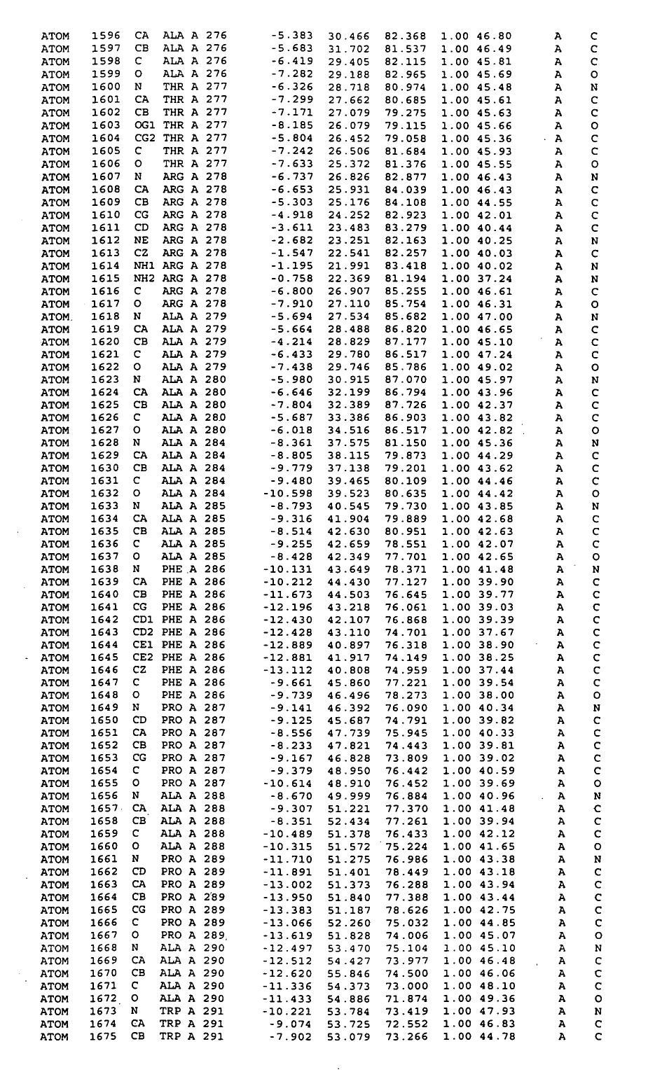

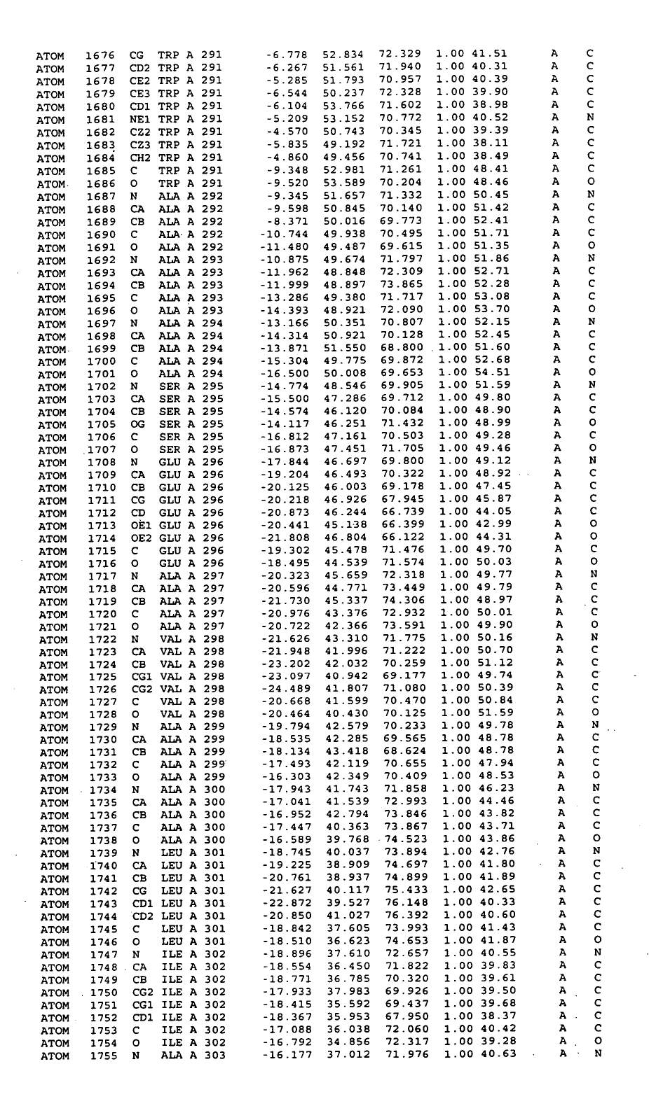

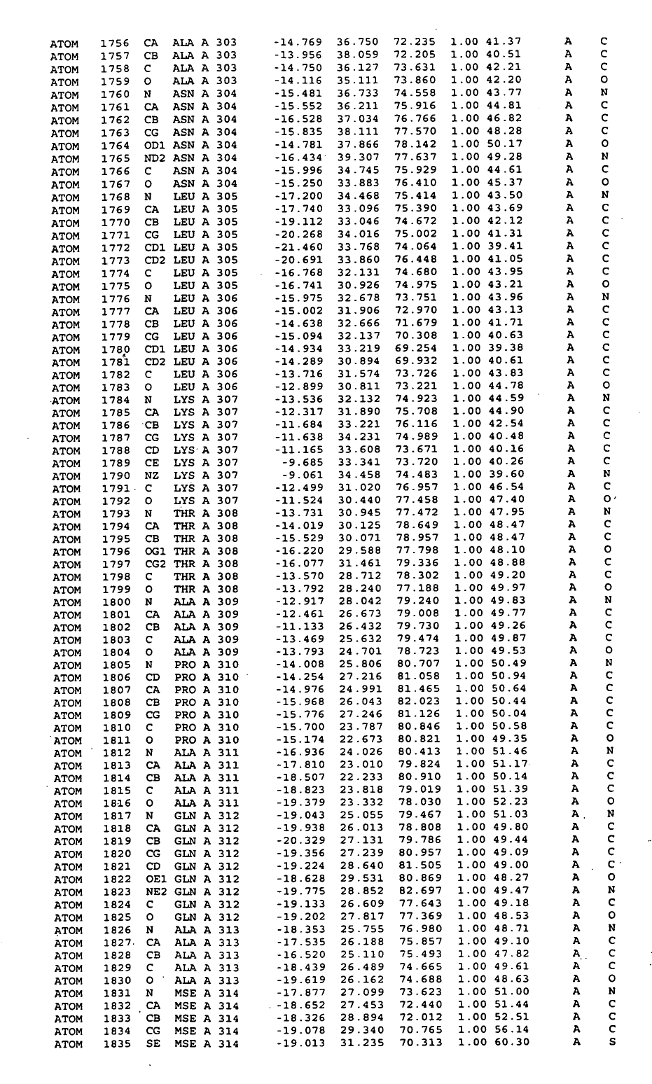

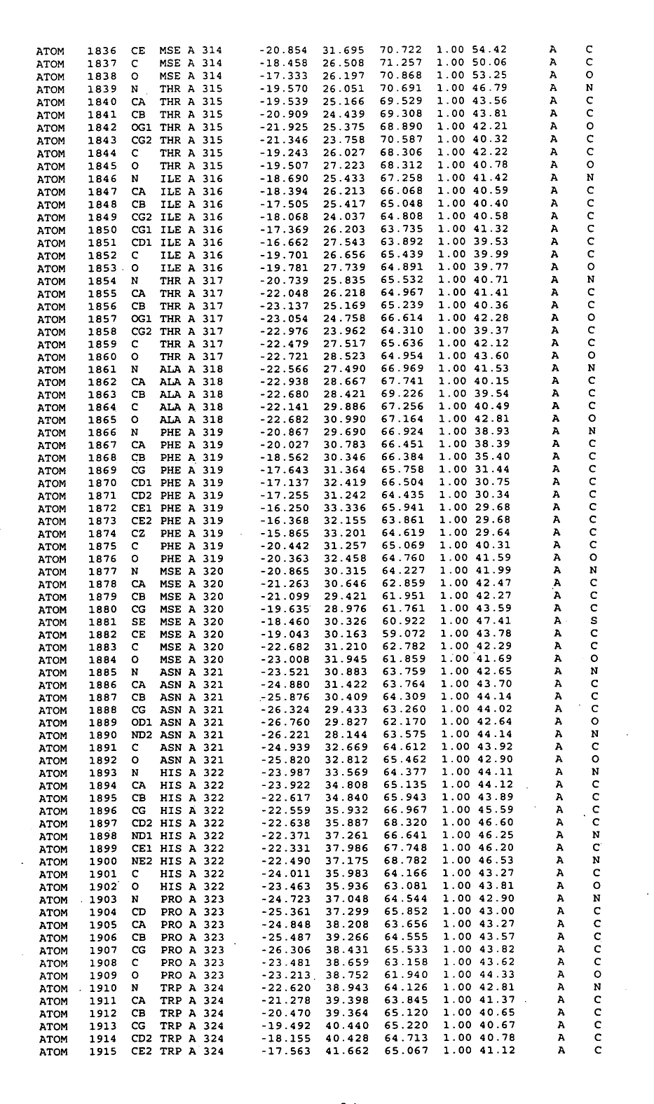

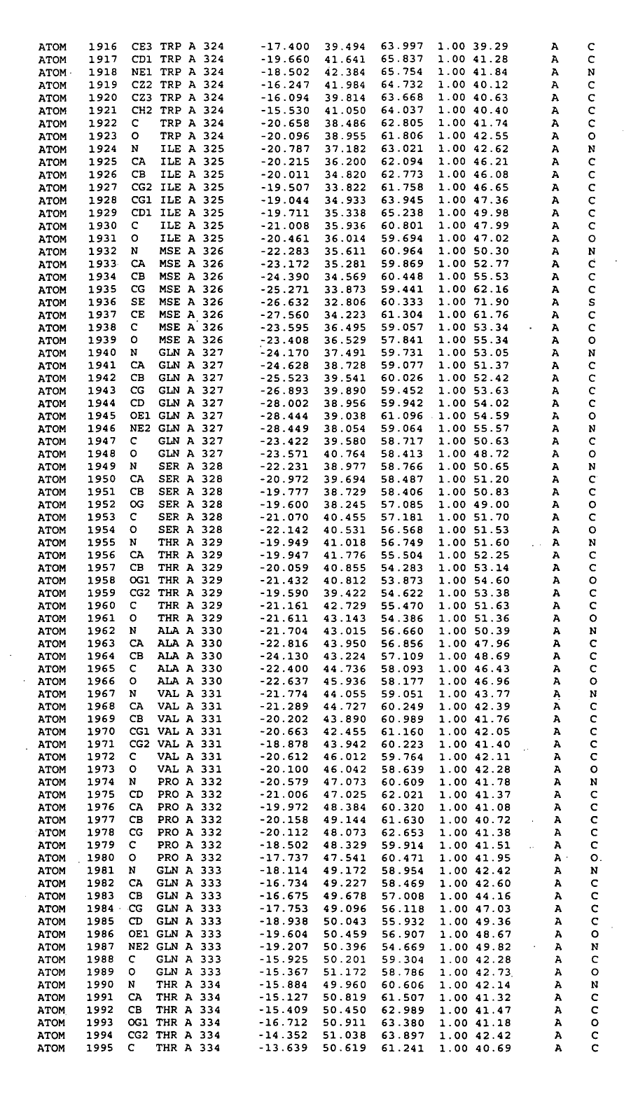

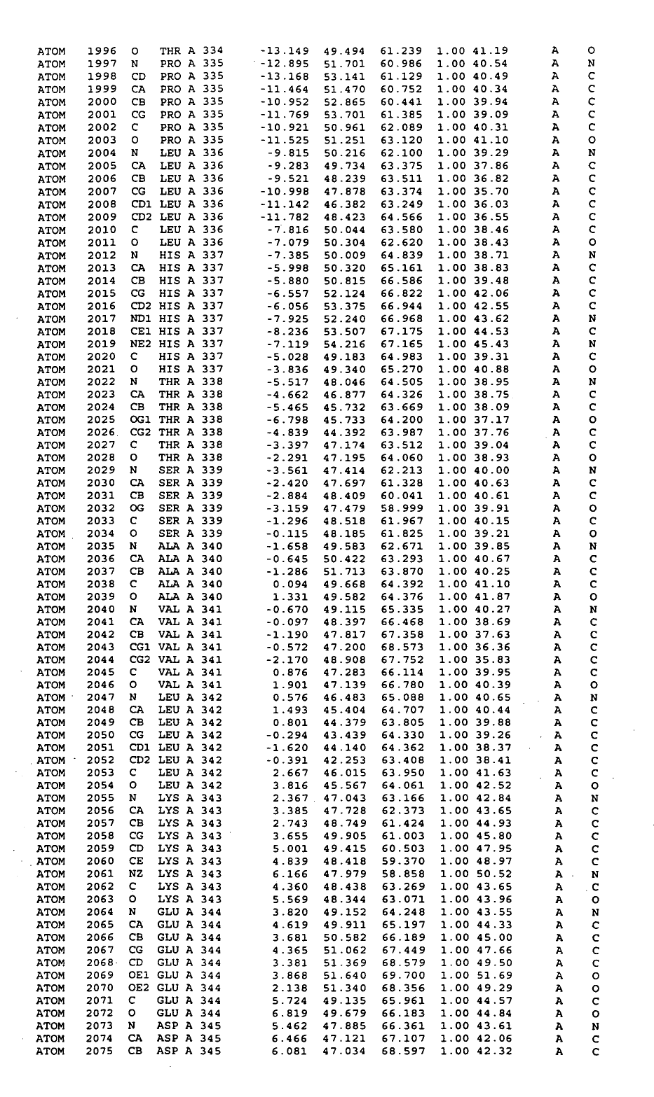

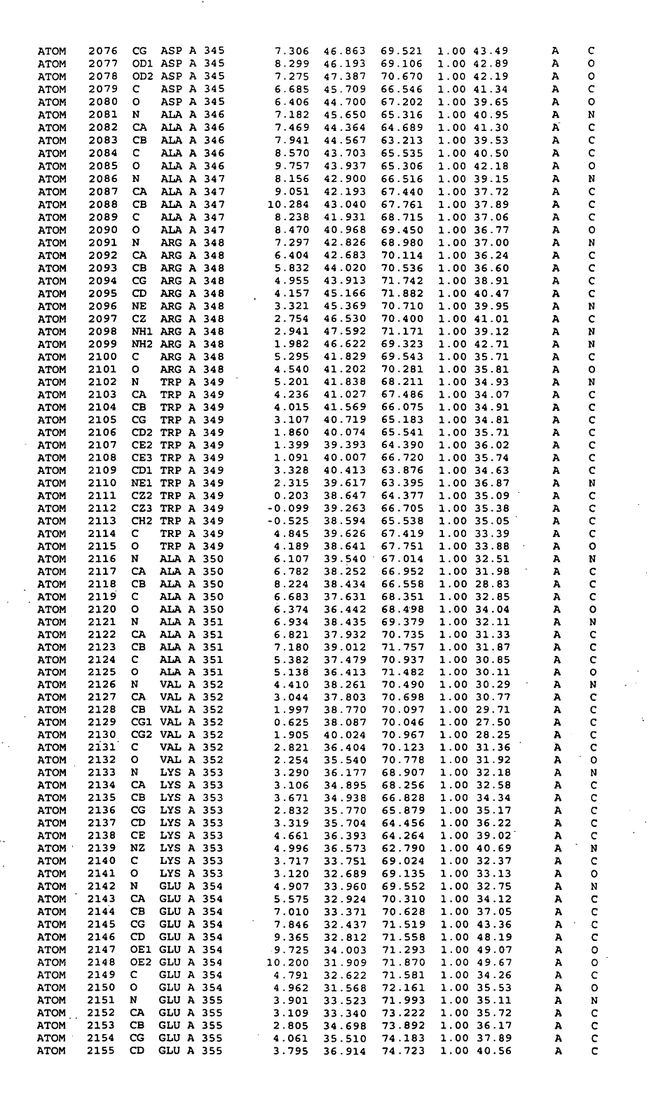

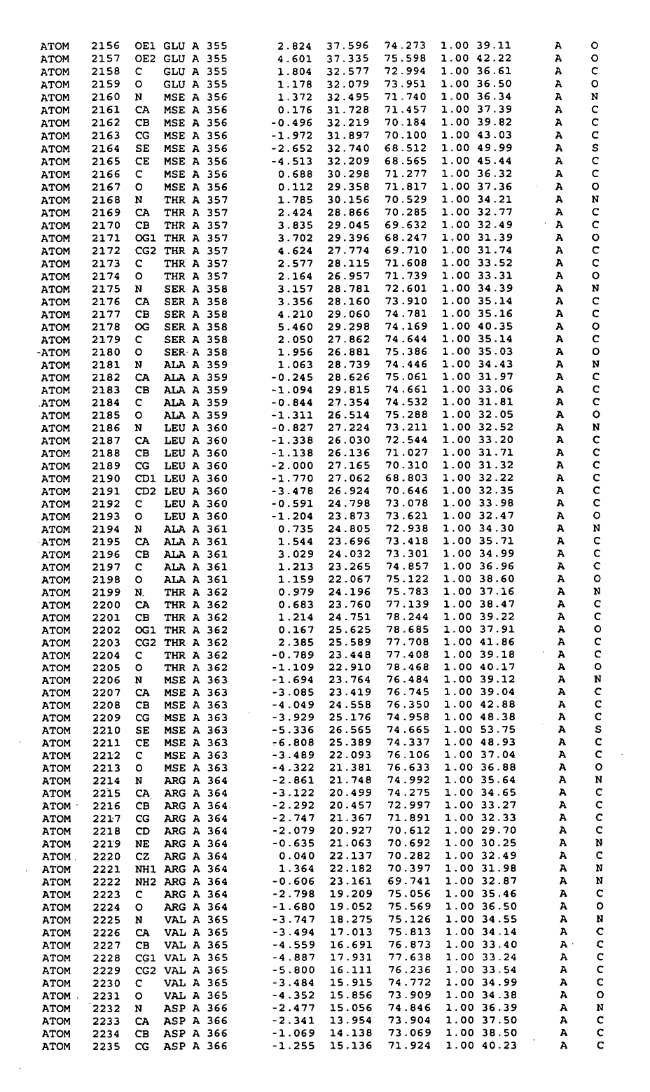

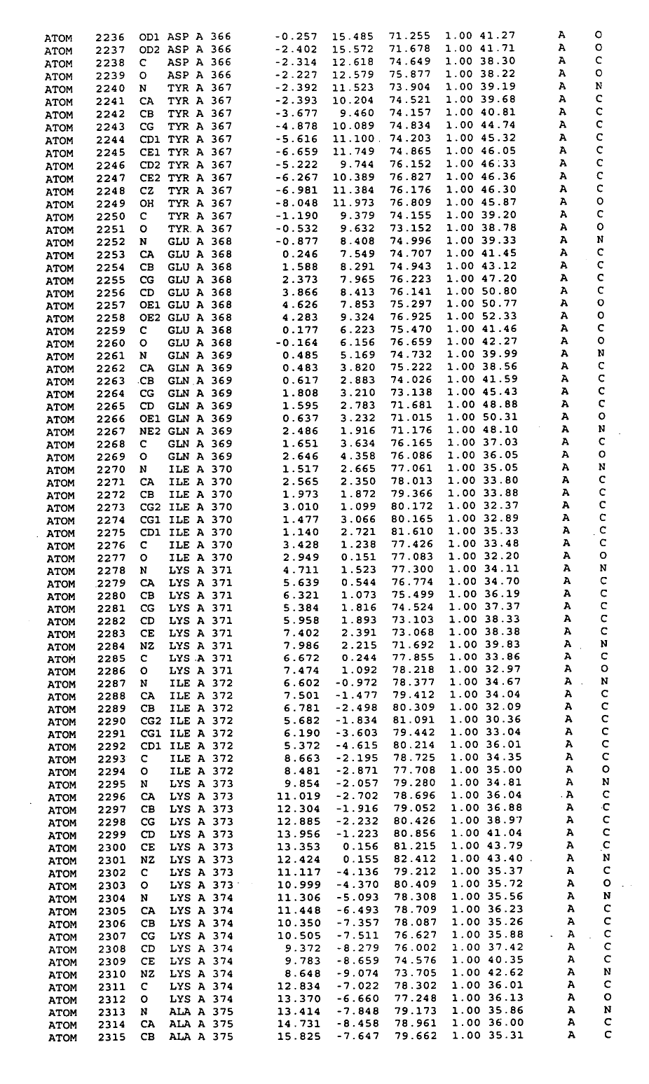

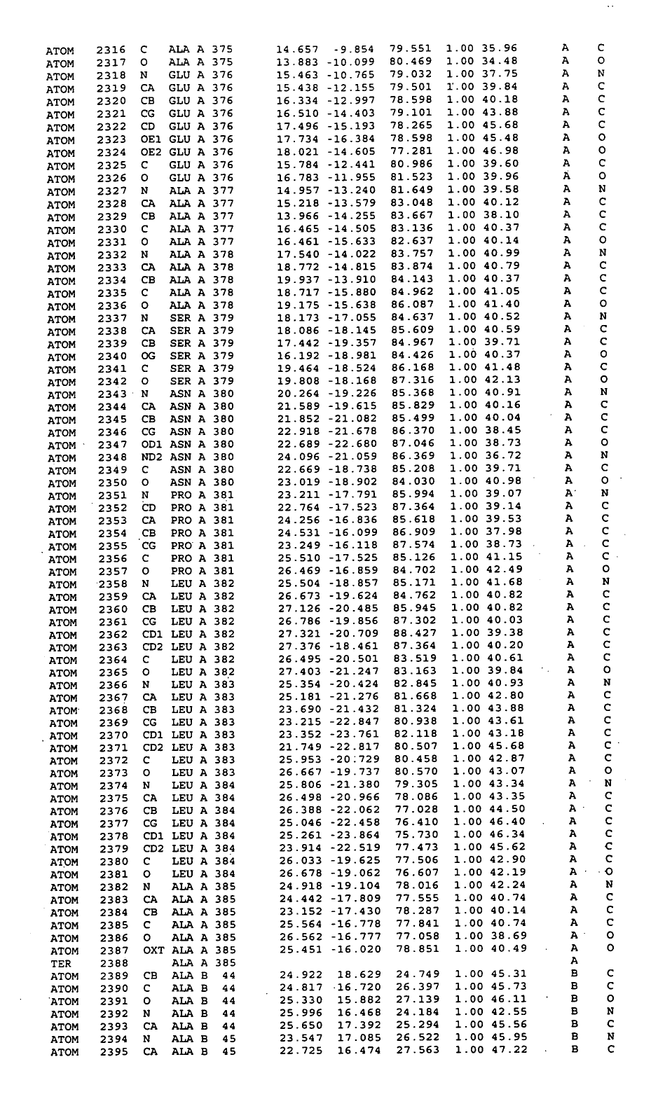

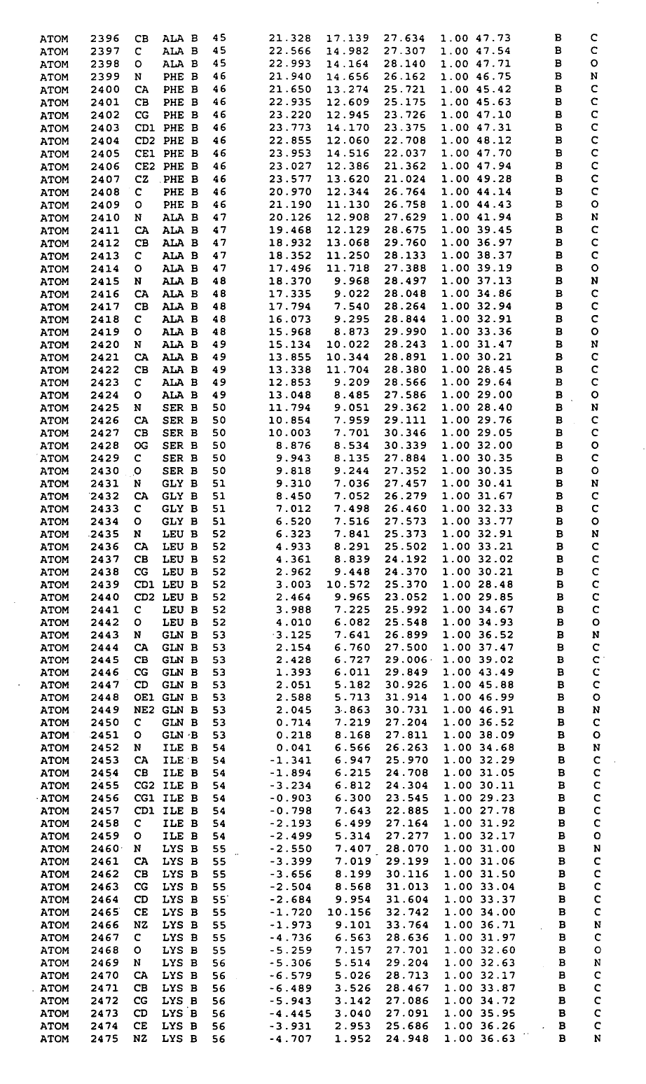

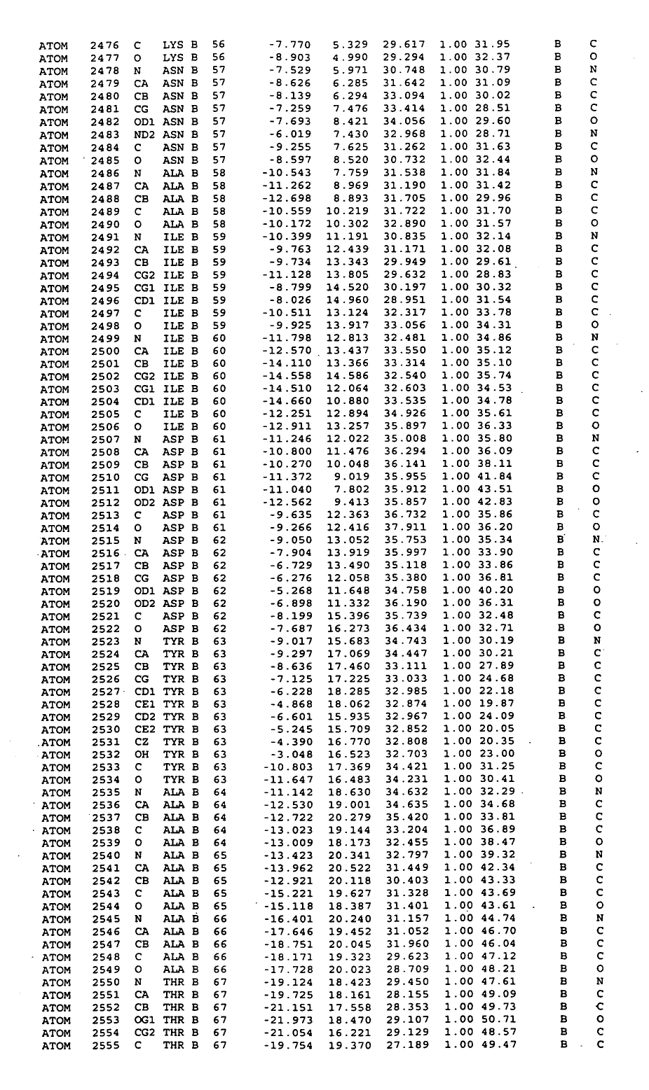

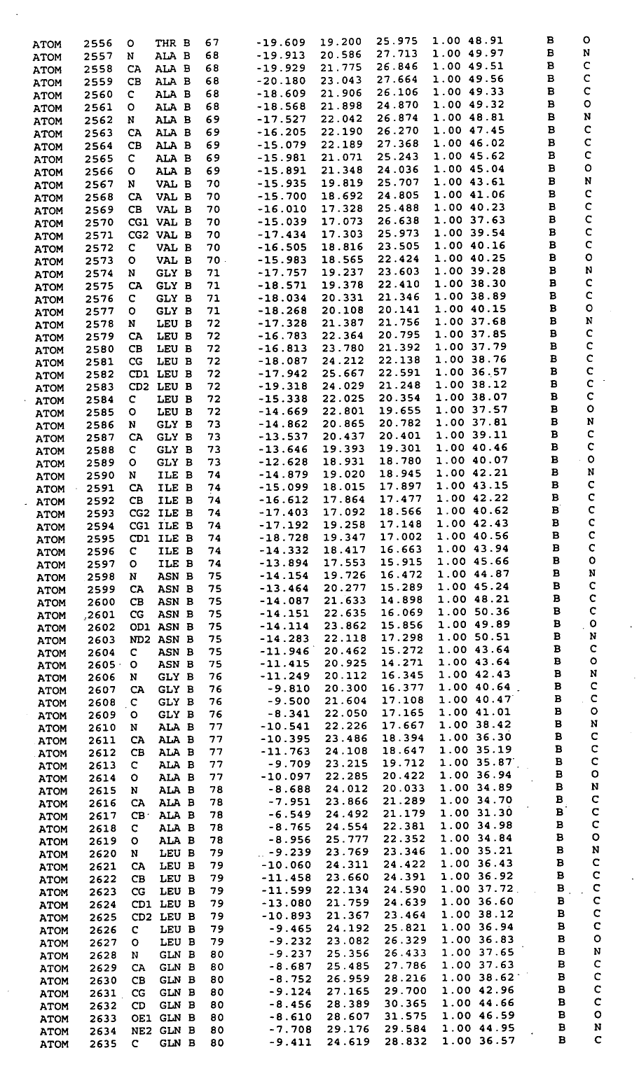

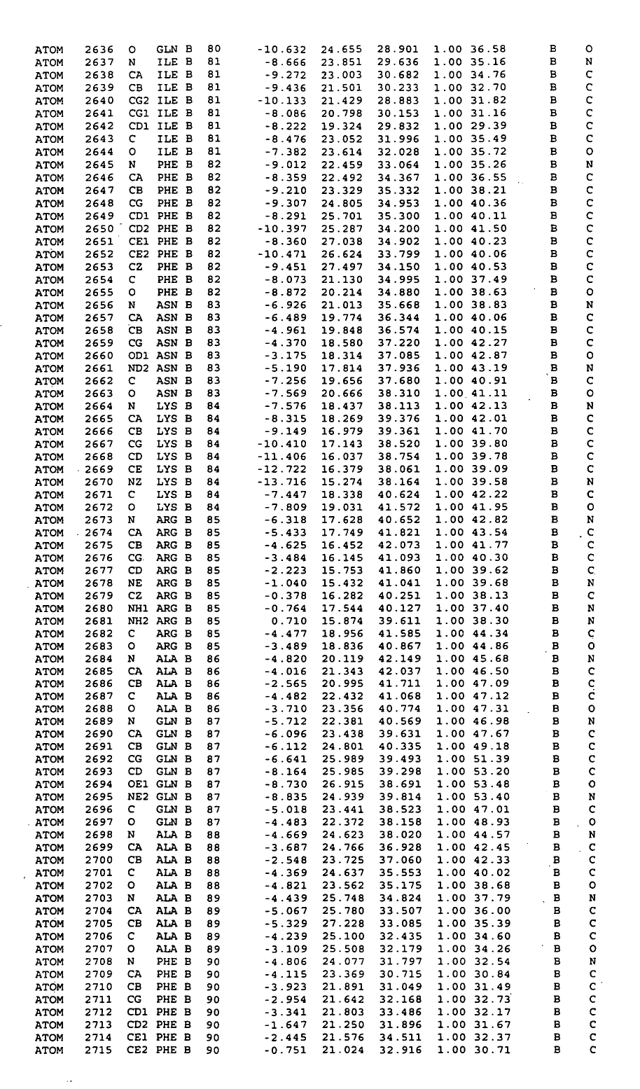

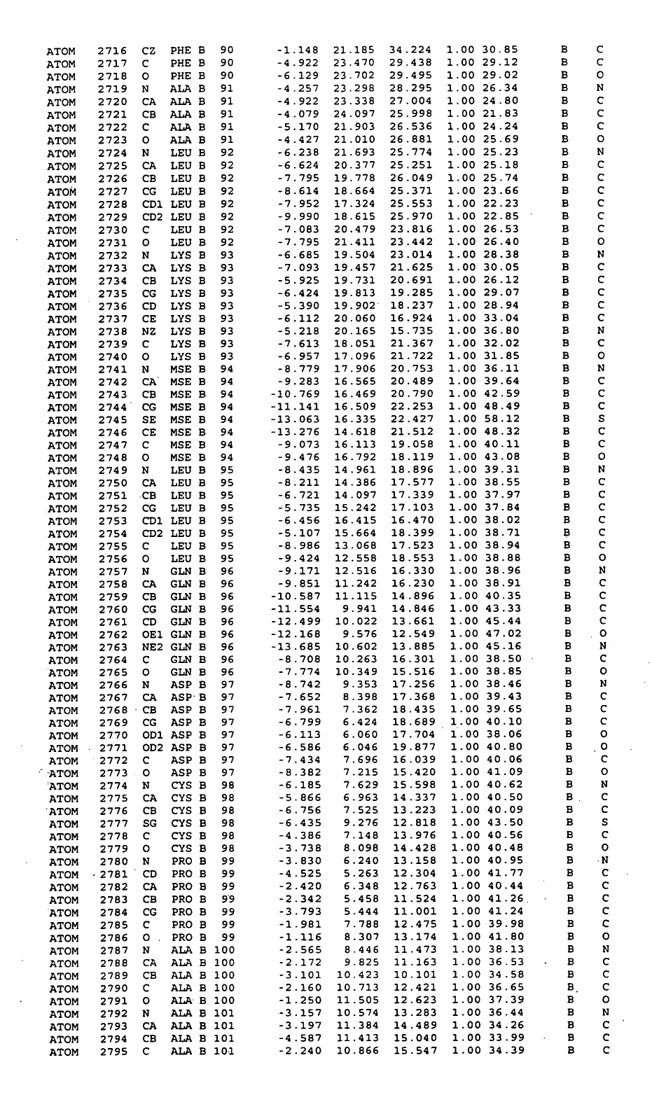

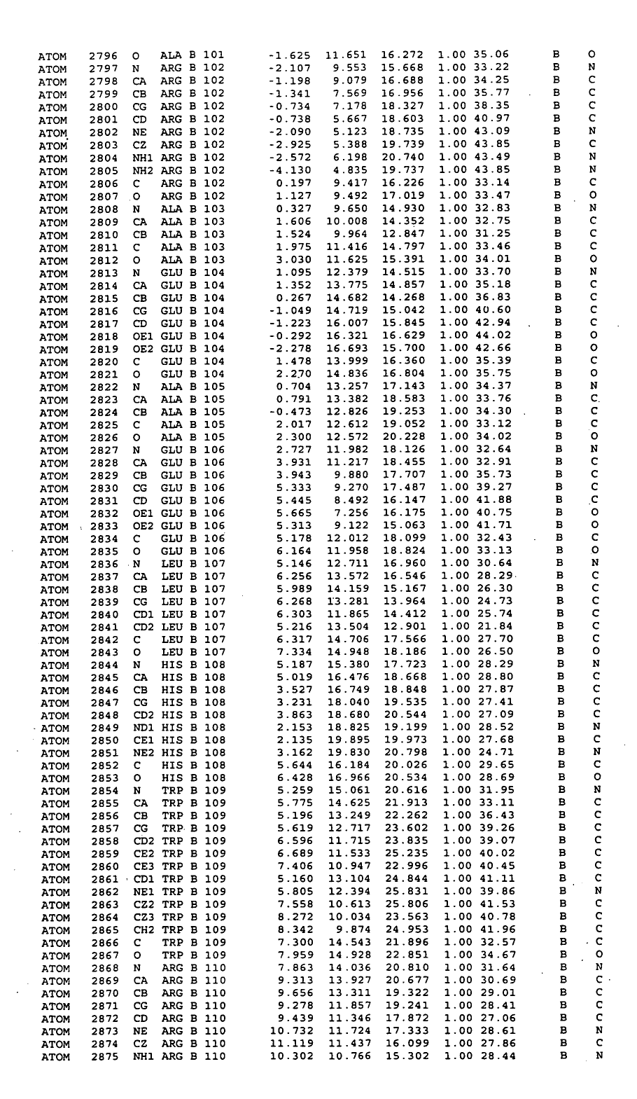

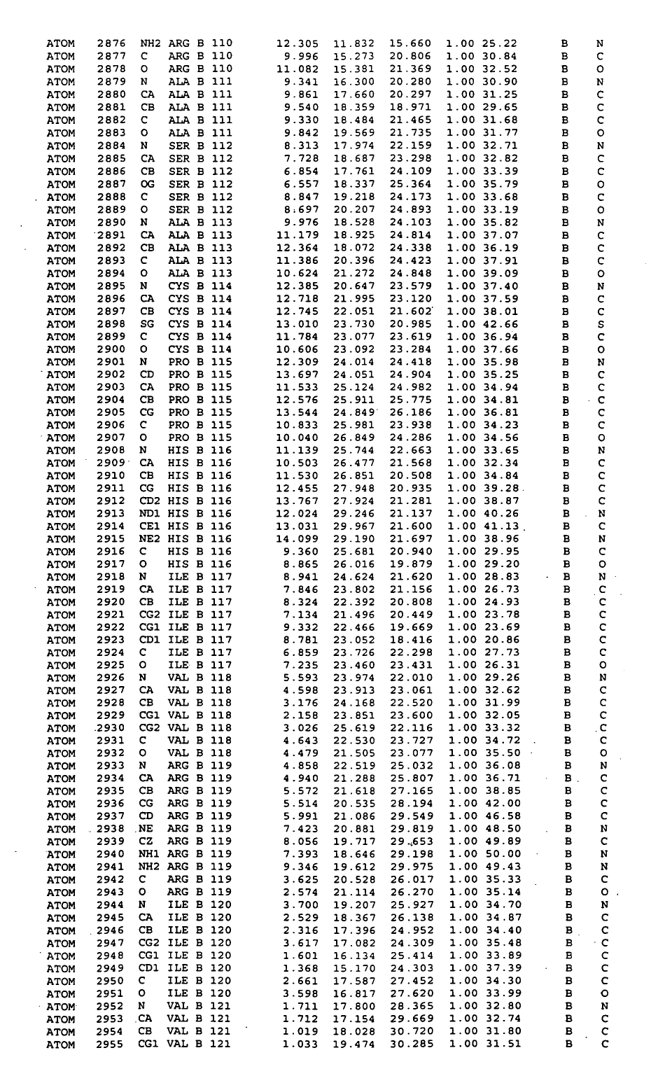

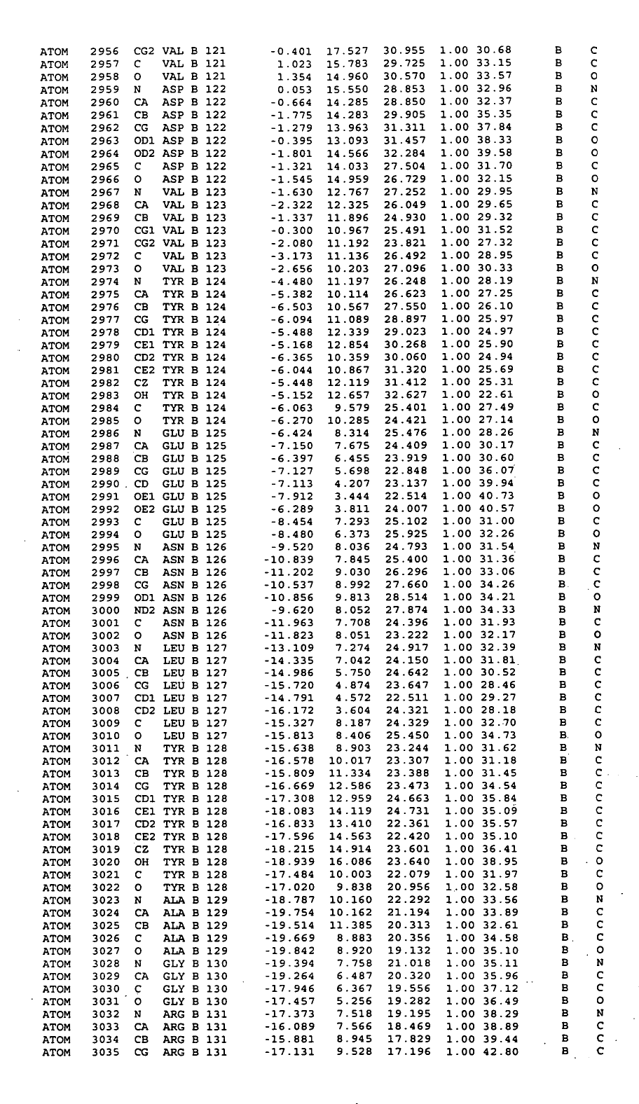

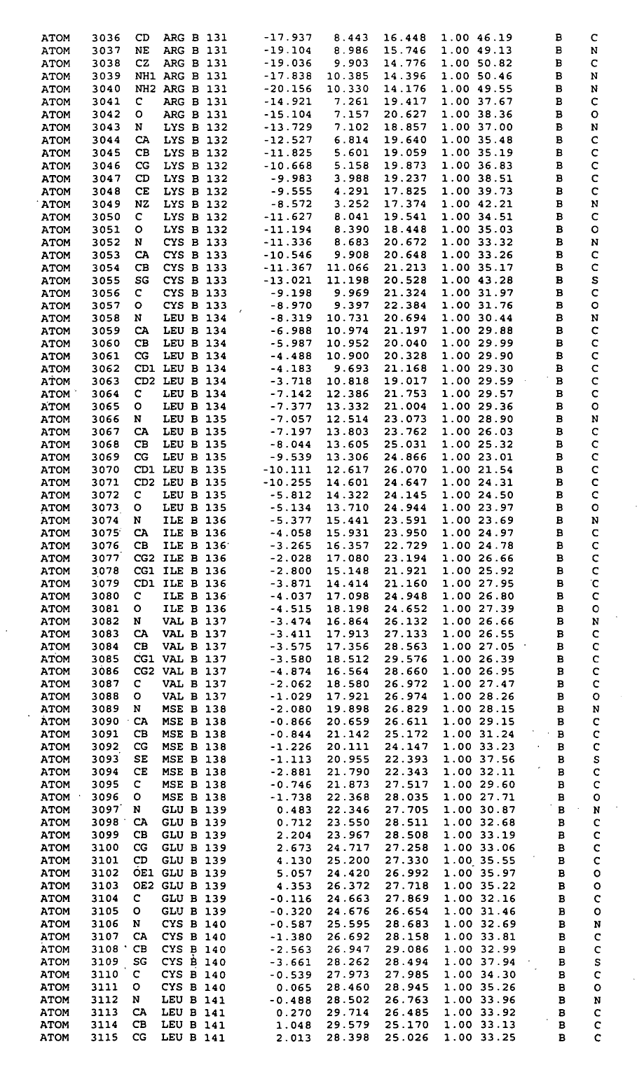

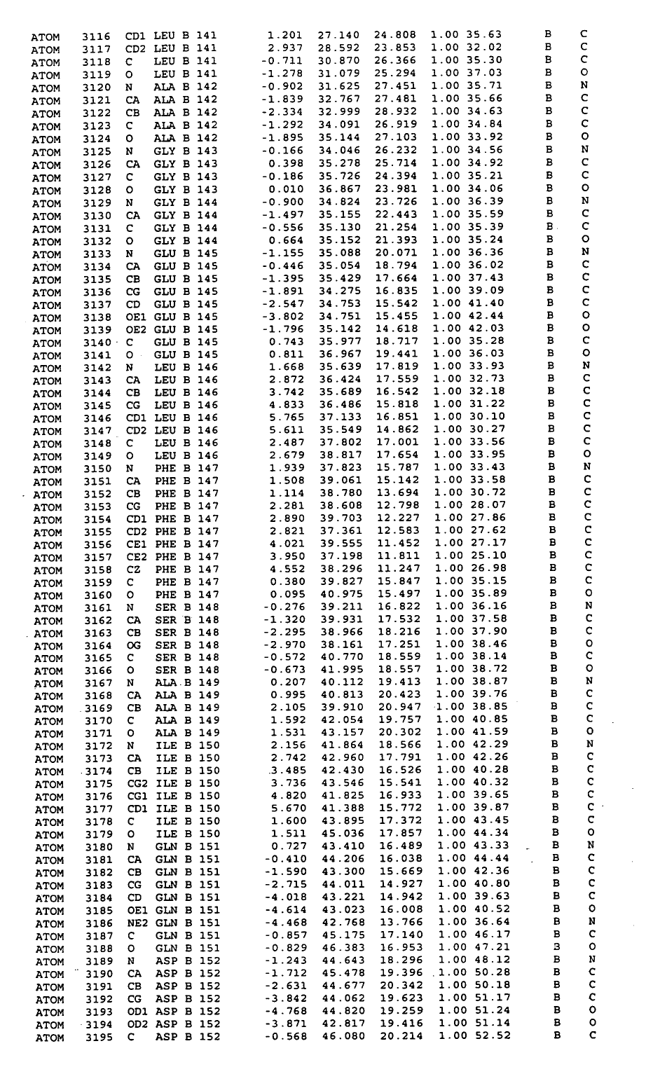

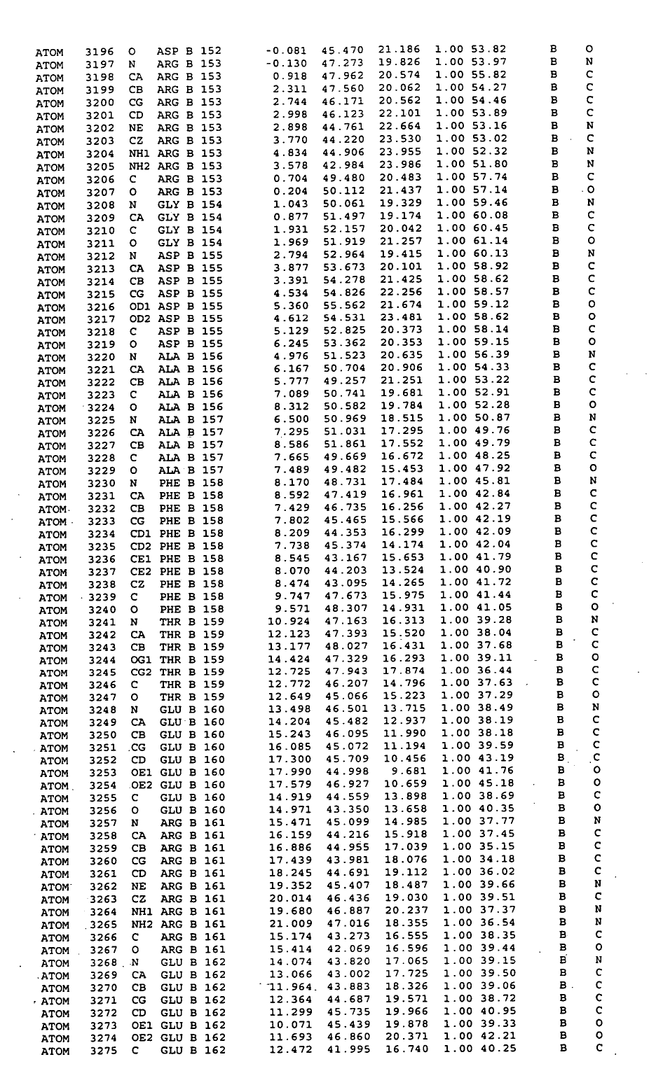

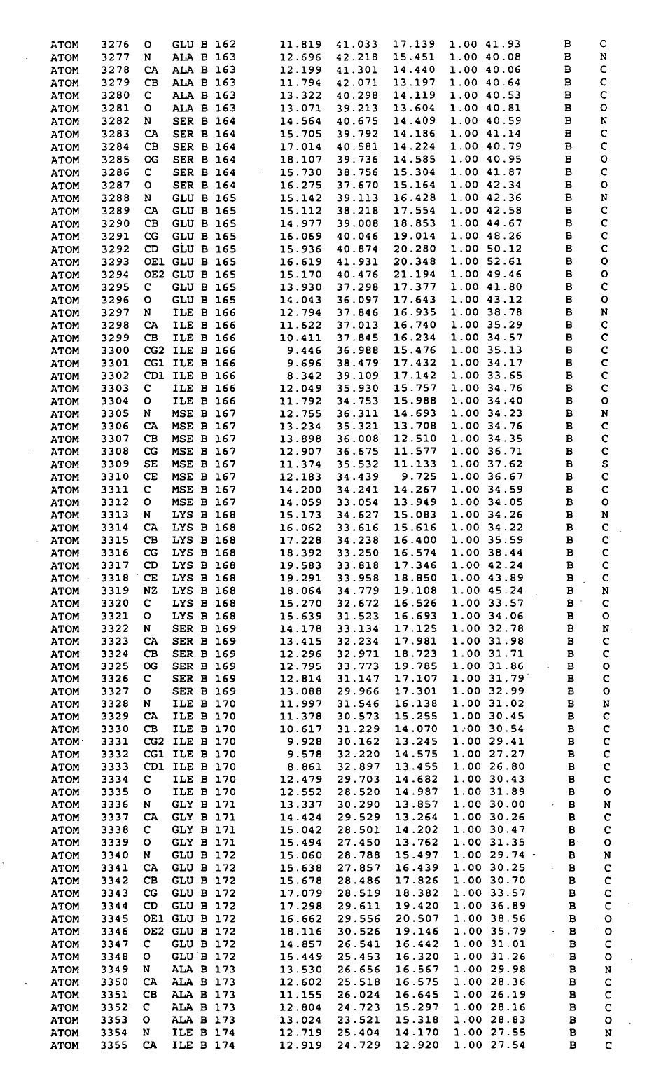

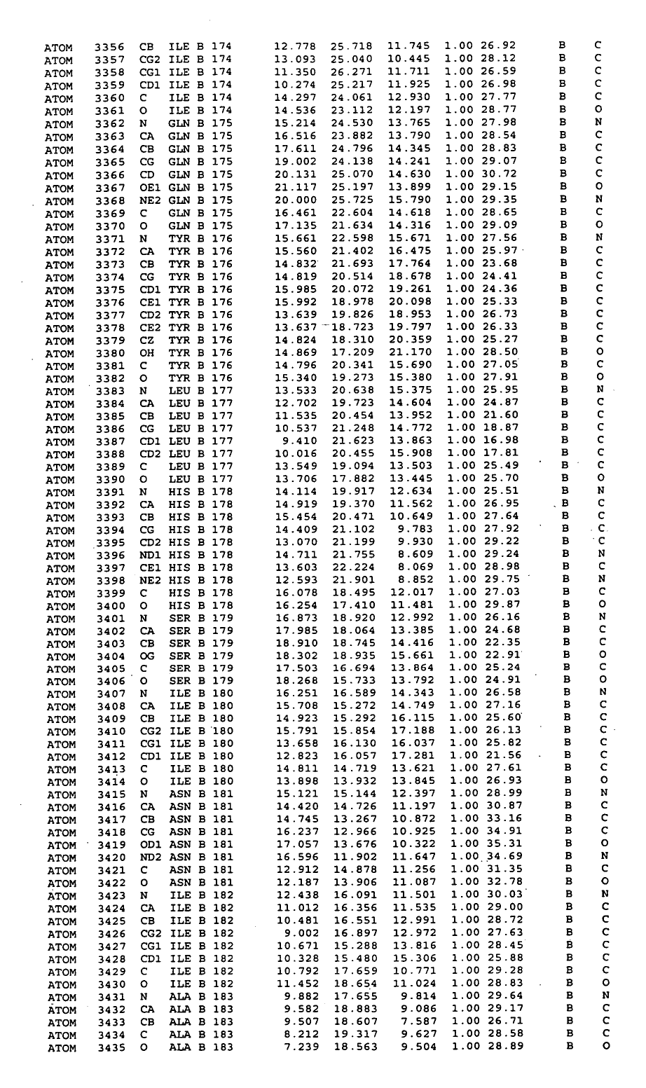

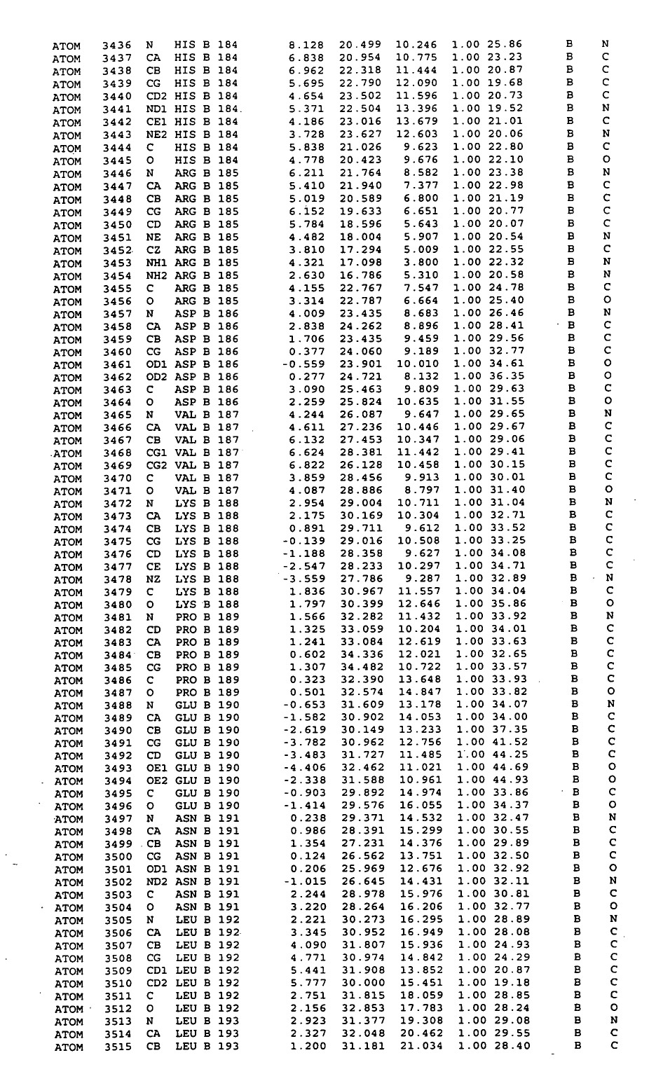

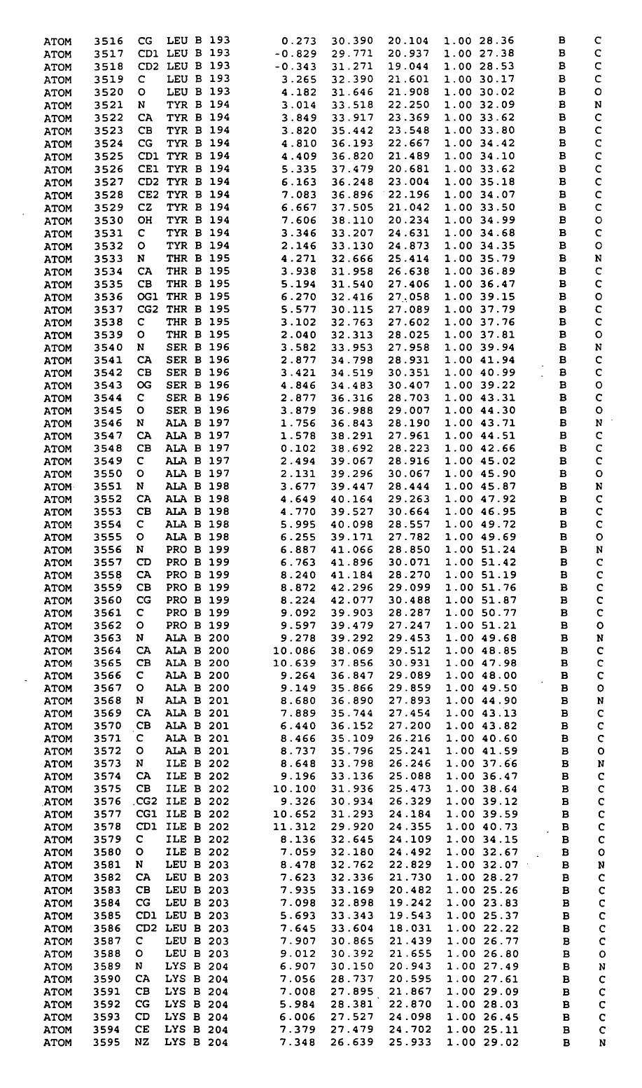

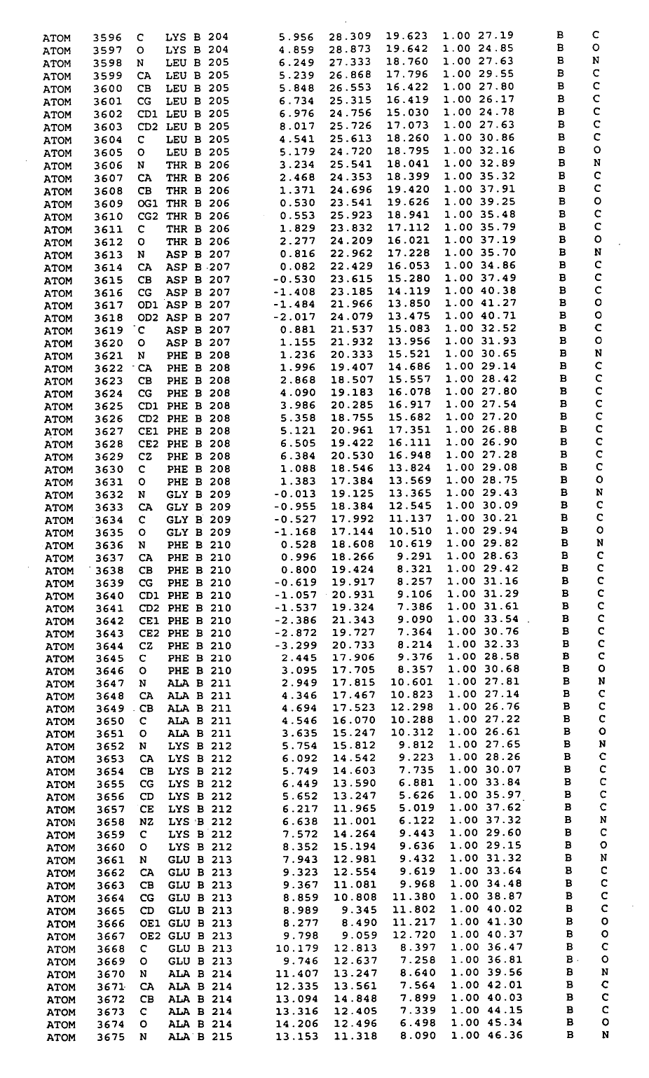

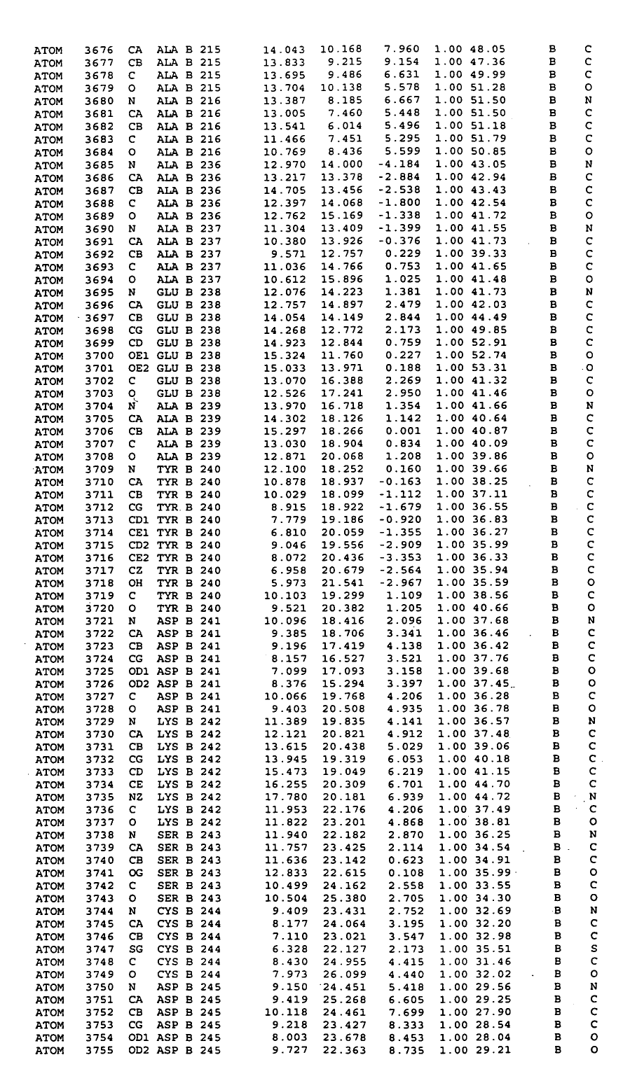

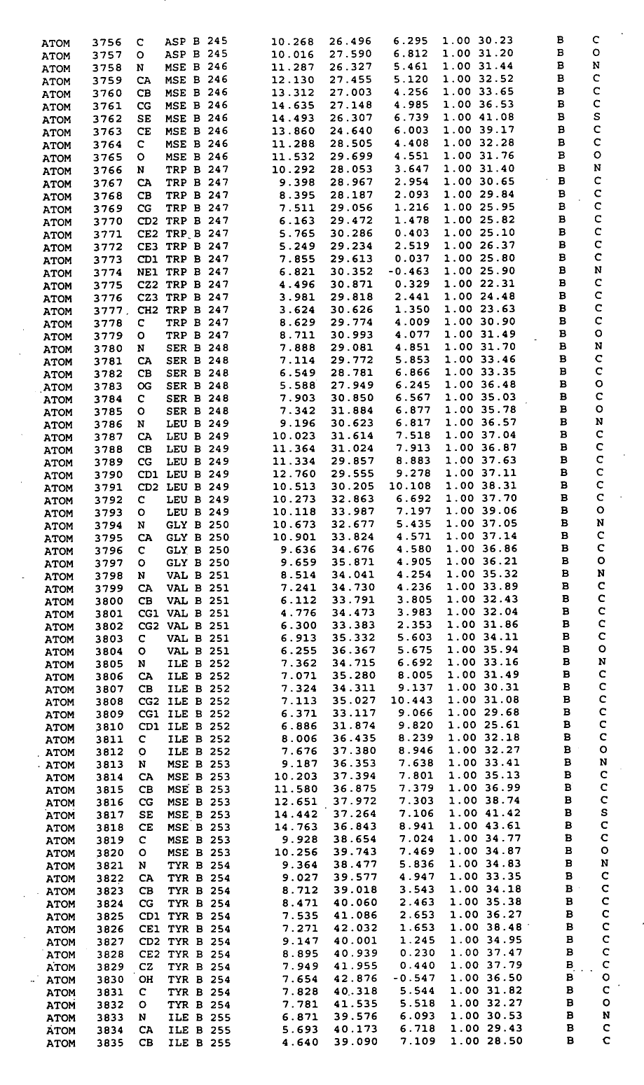

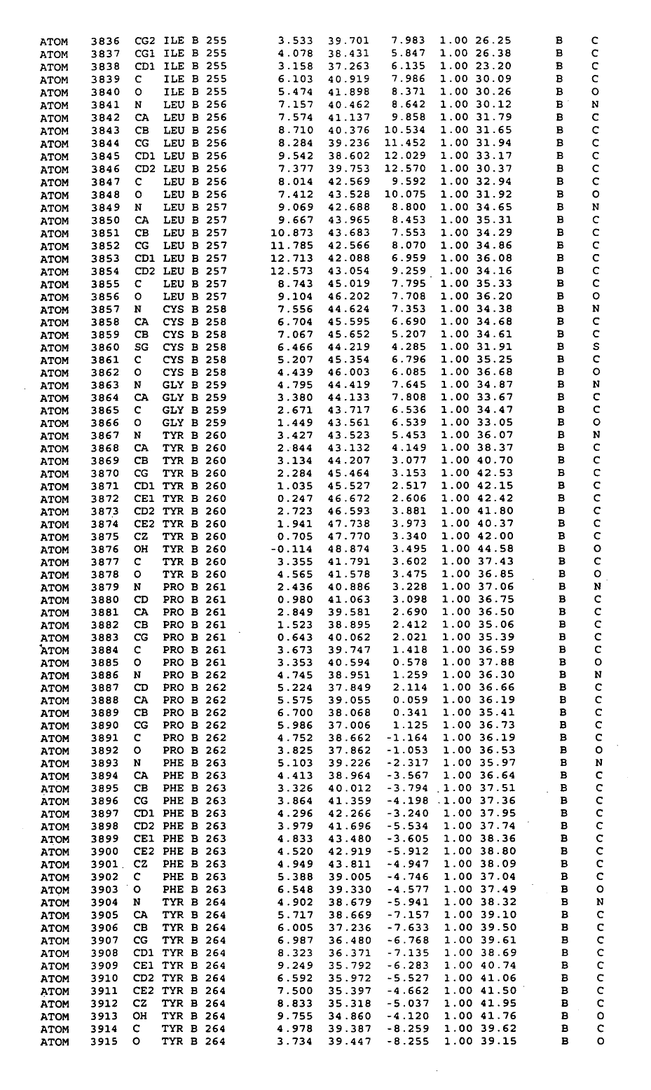

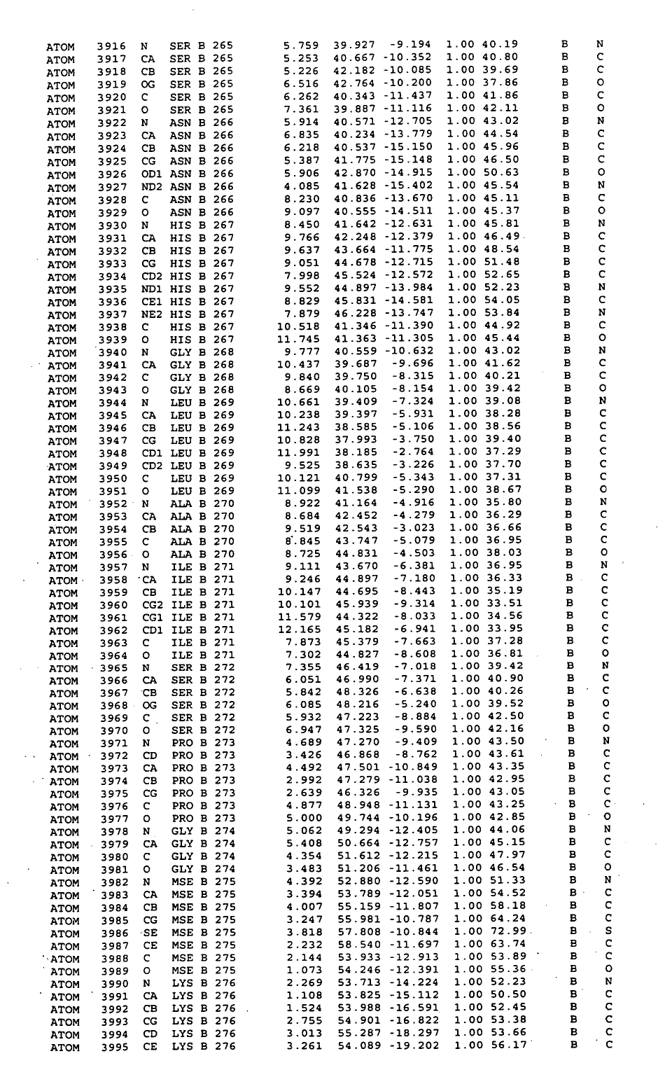

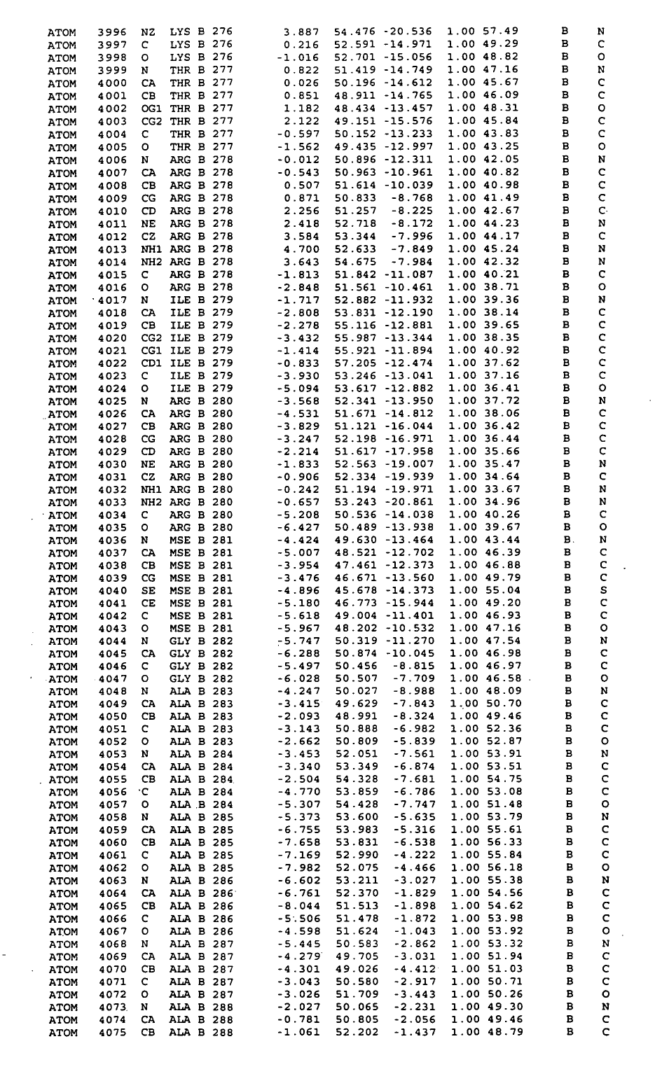

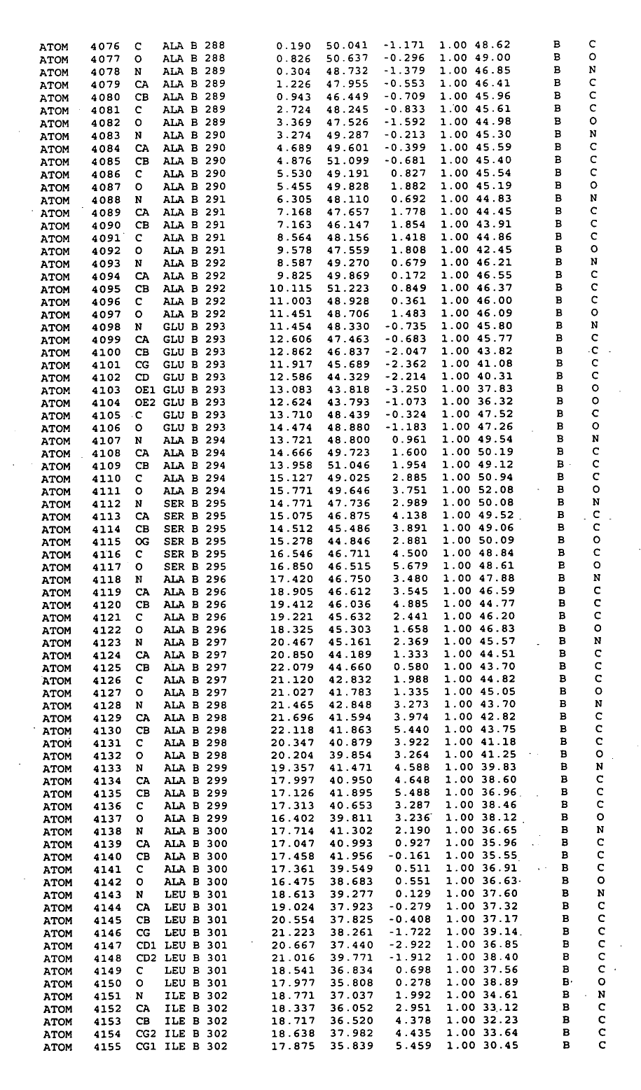

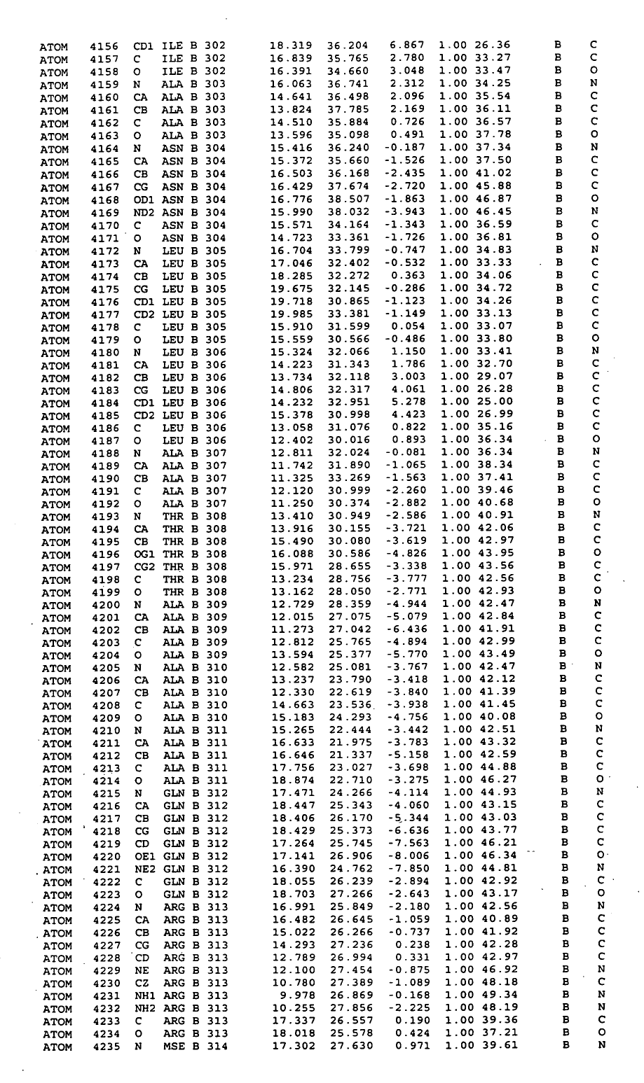

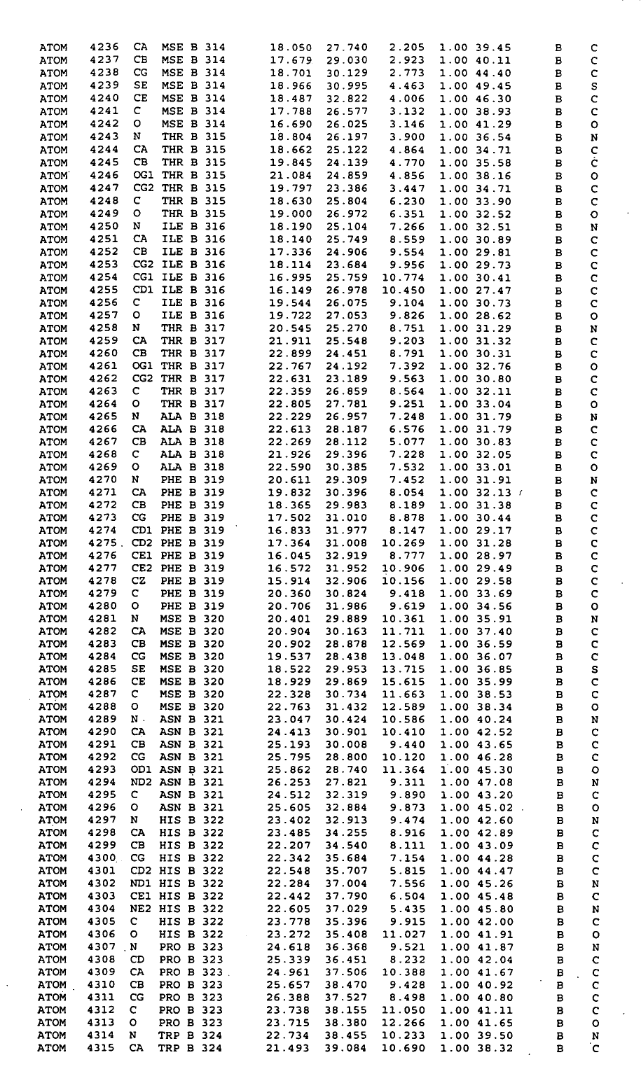

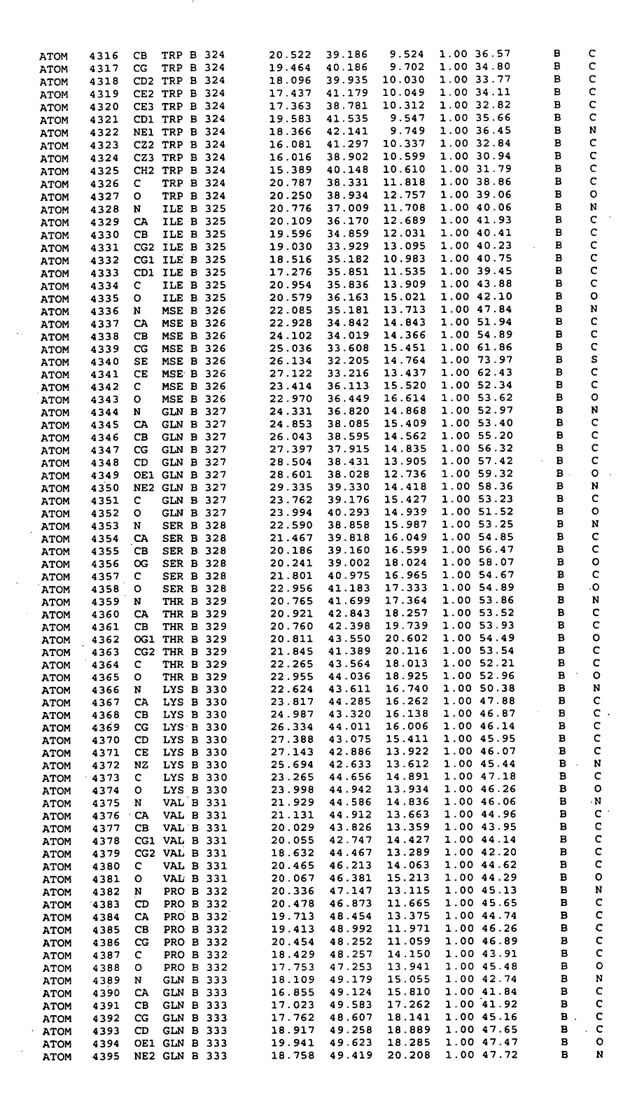

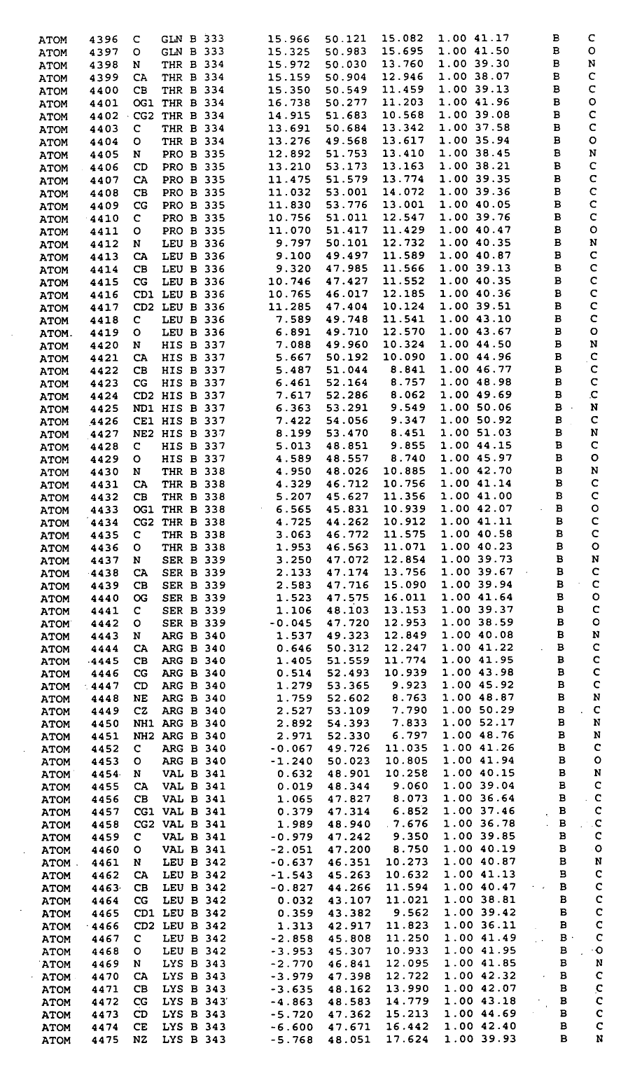

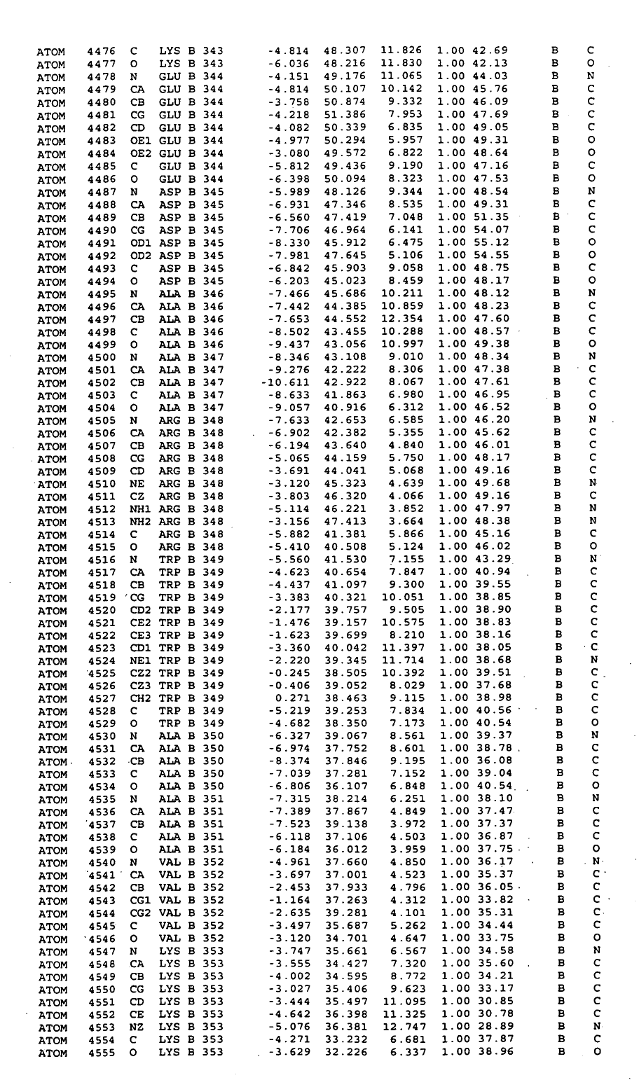

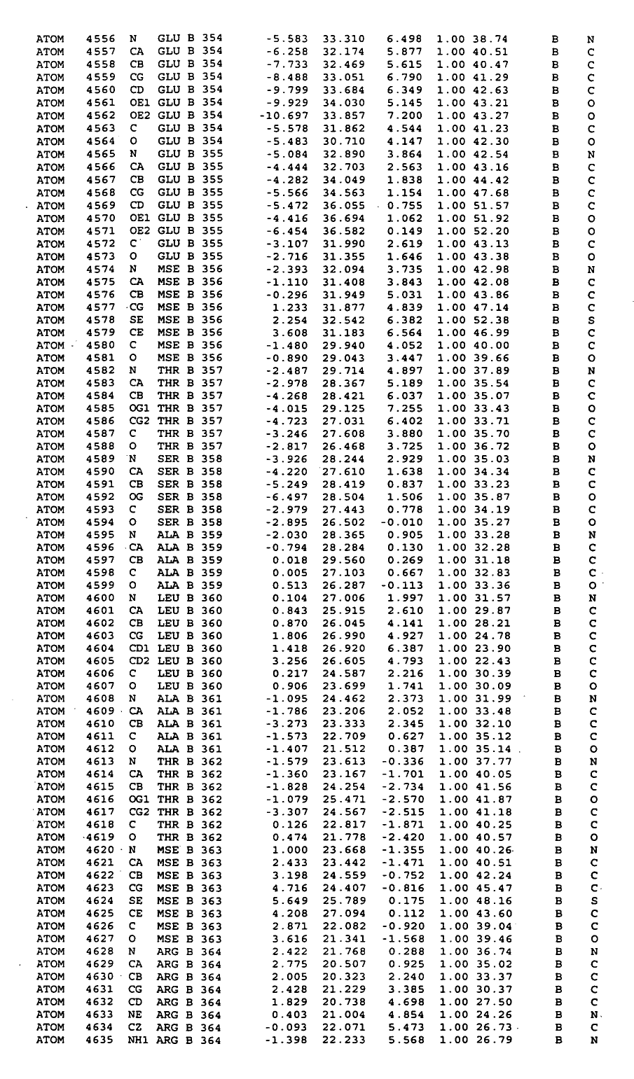

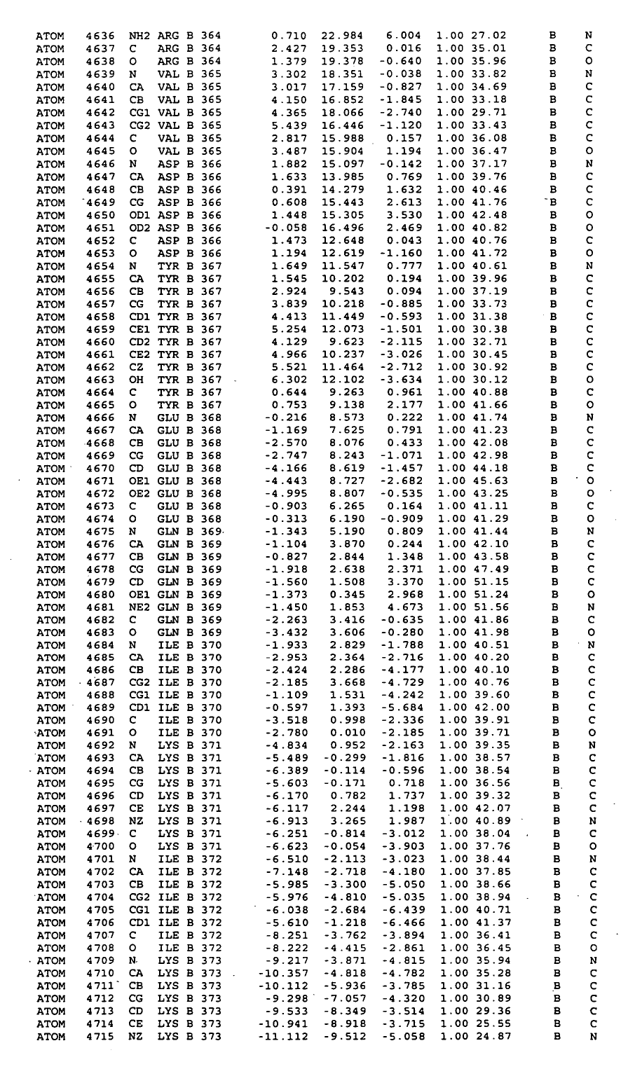

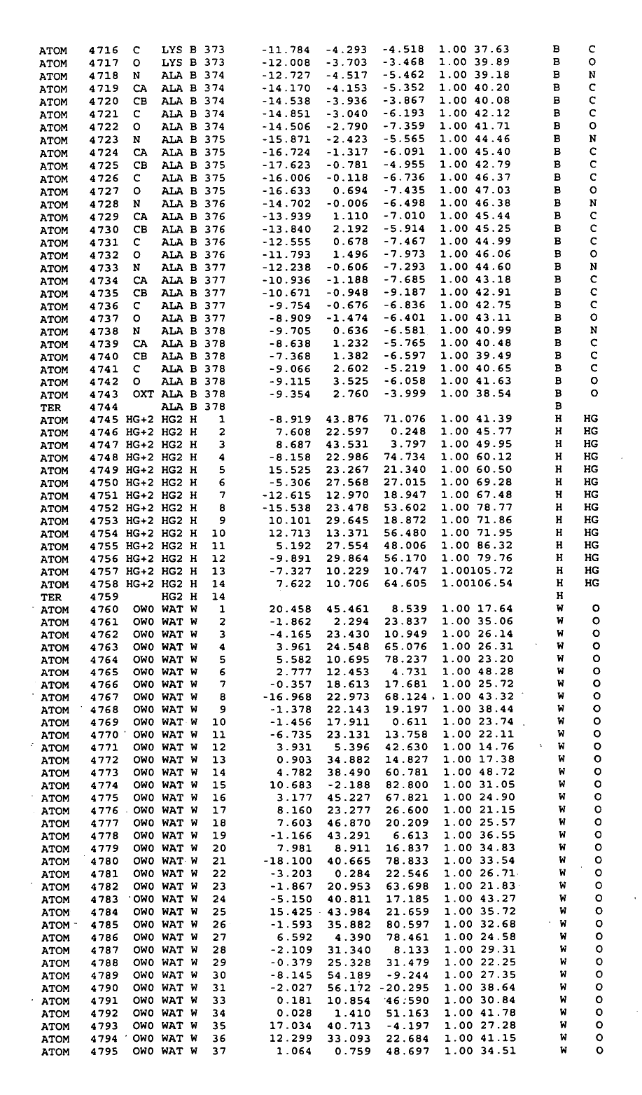

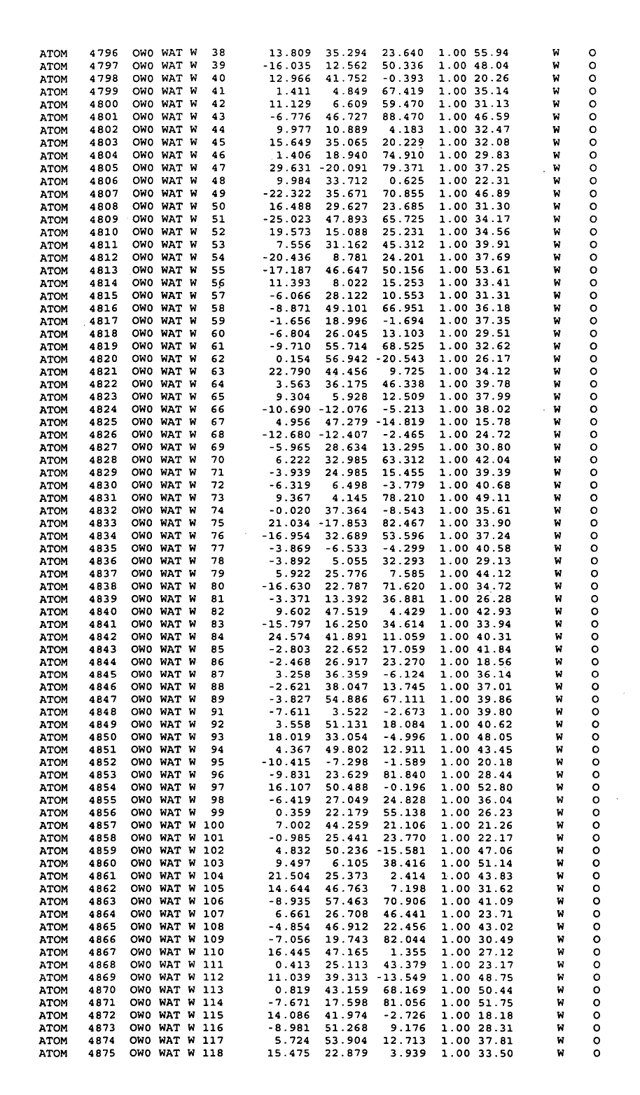

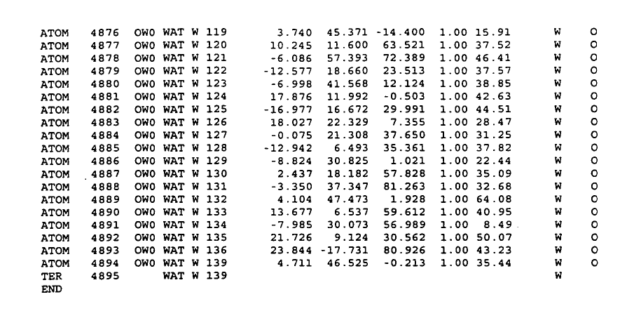

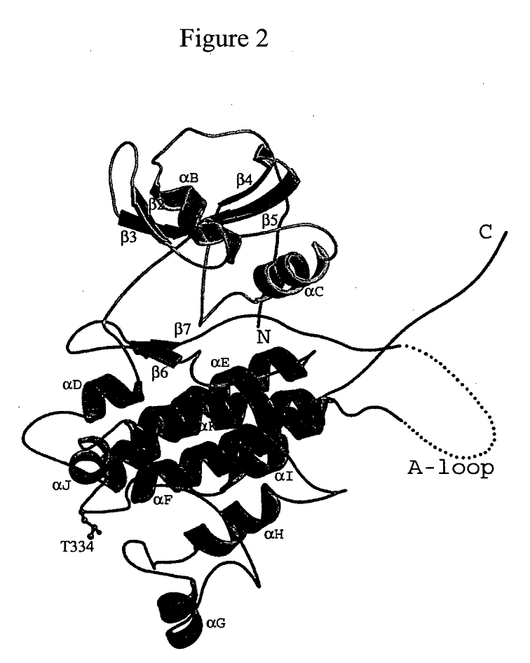

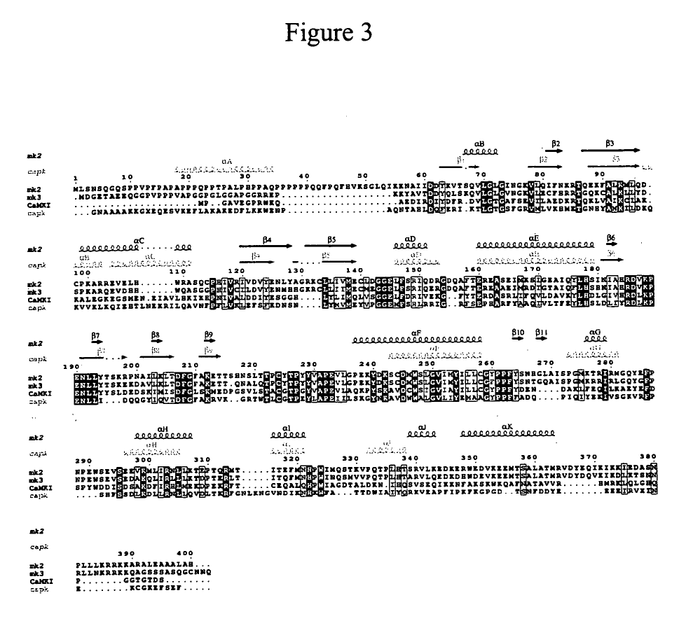

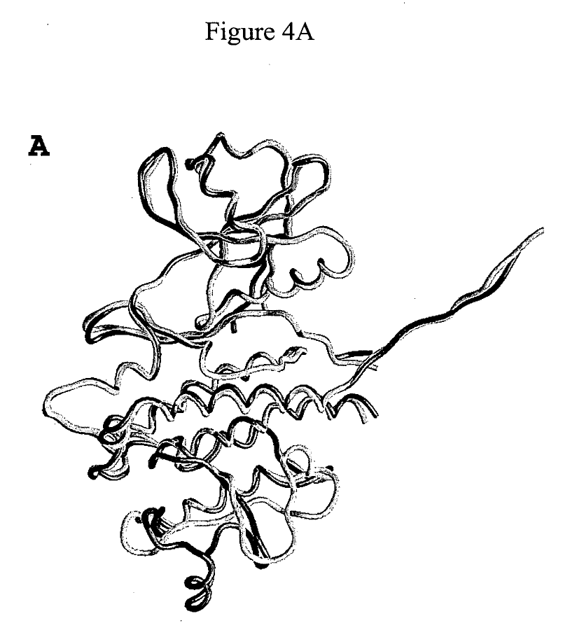

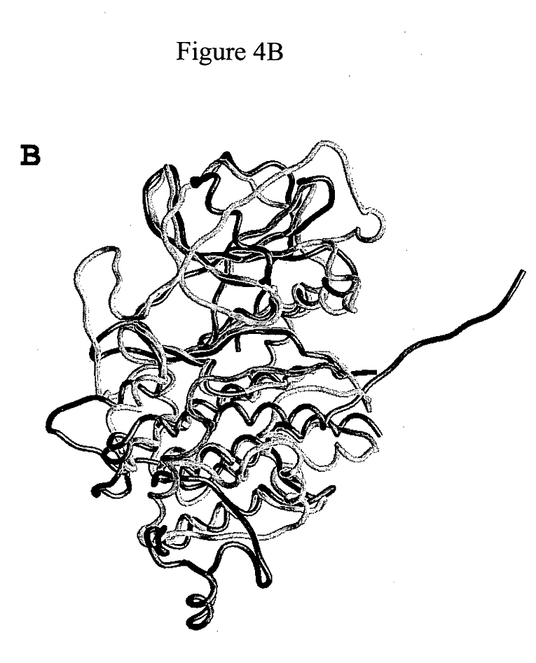

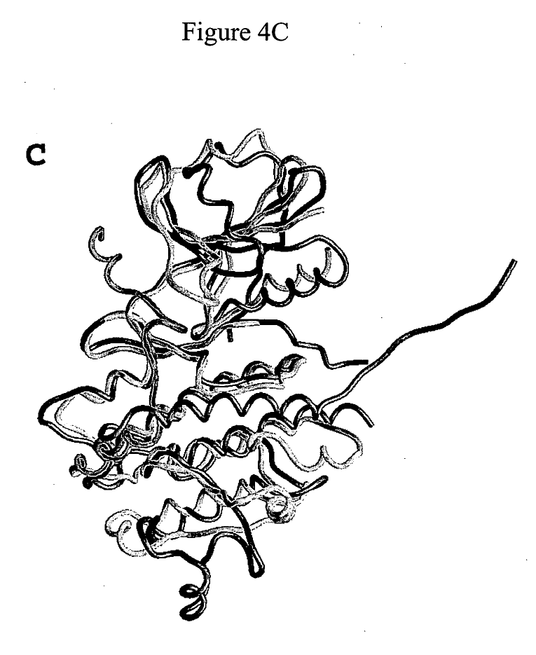

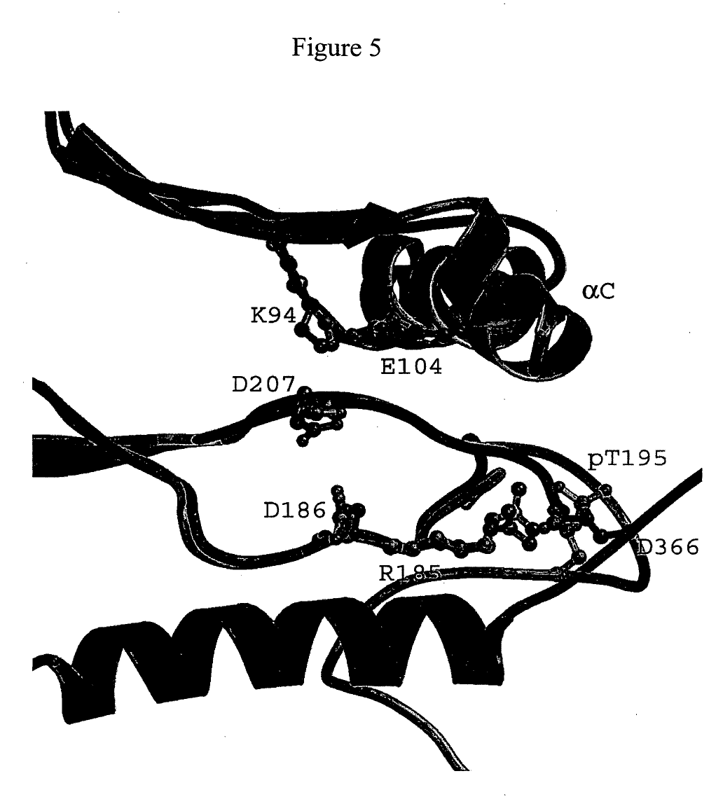

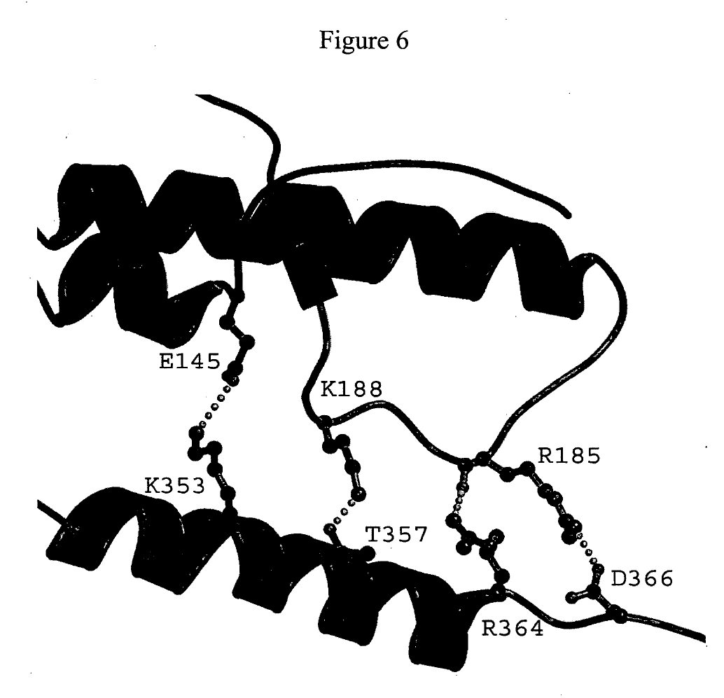

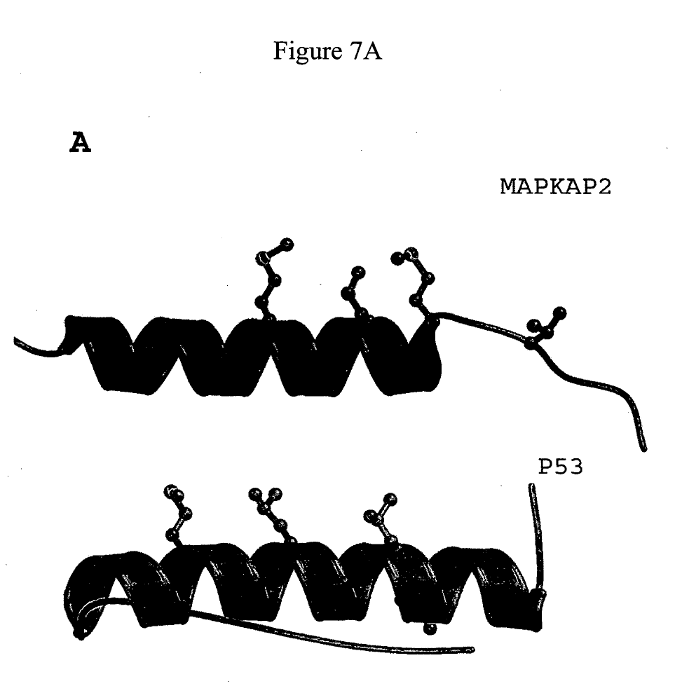

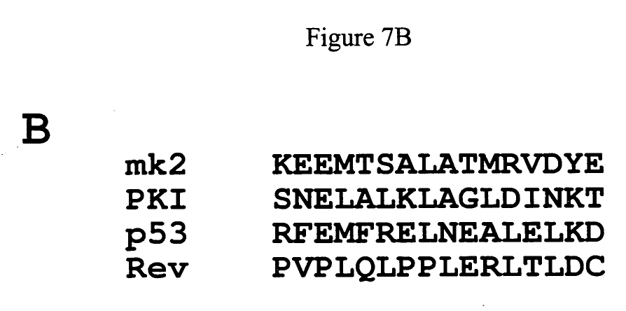

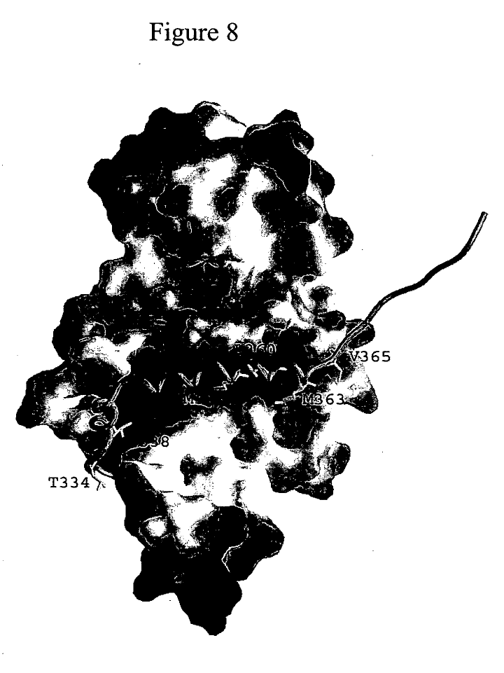

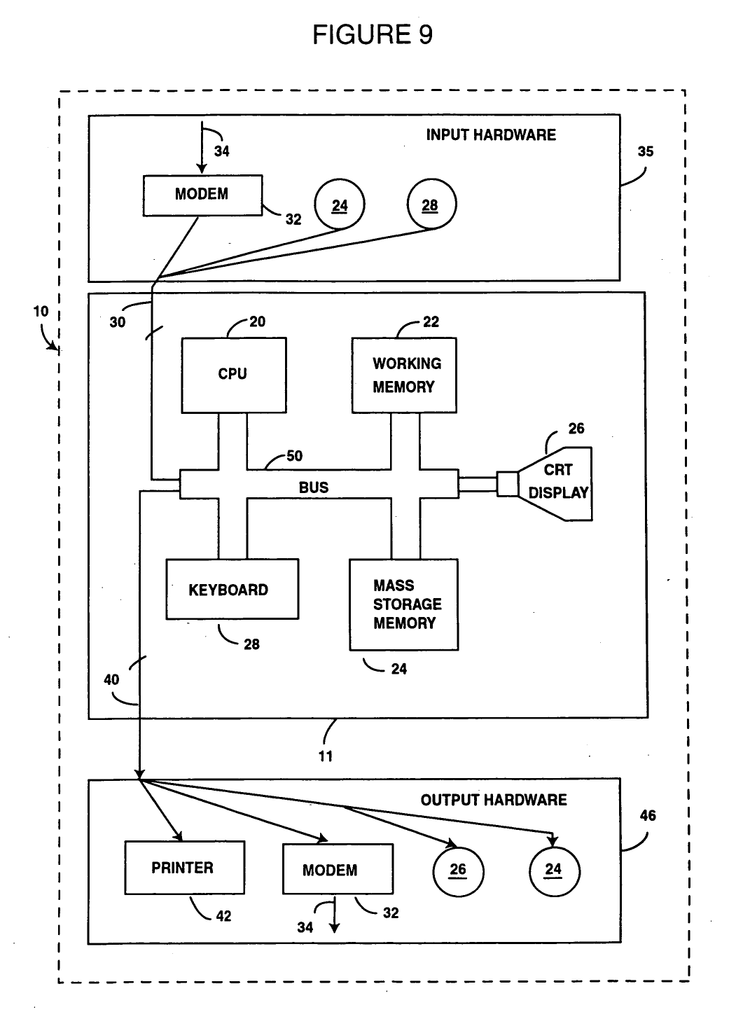

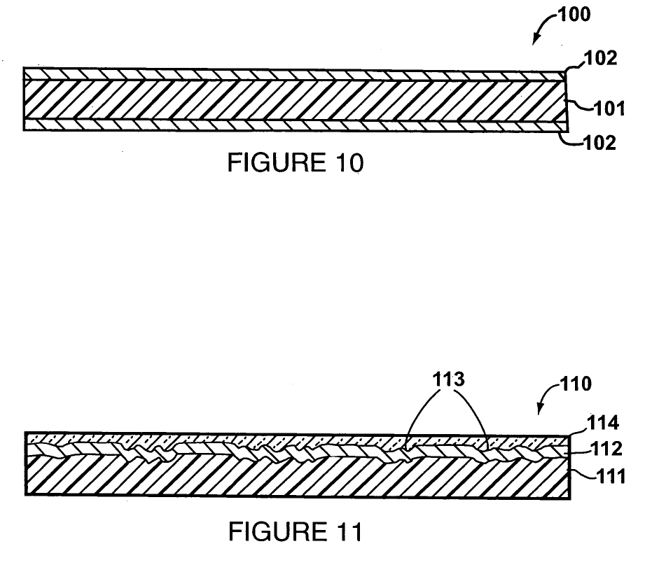

The term “MAPKAPK2-like” refers to all or a portion of a molecule or molecular complex that has a commonality of shape to all or a portion of the MAPKAPK2 protein. In the MAPKAPK2-like ATP binding pocket, the commonality of shape is defined by a root mean square deviation of the structure coordinates of the backbone atoms between the amino acids in the MAPKAPK2-like ATP-binding pocket and the amino acids in the MAPKAPK2 ATP-binding pocket (as set forth in FIG. 1). Compared to an amino acid in the MAPKAPK2 ATP-binding pocket, the corresponding amino acids in the MAPKAPK2-like ATP-binding pocket may or may not be identical.

The term “part of a MAPKAPK2 ATP-binding pocket” or “part of a MAPKAPK2-like ATP-binding pocket” refers to a portion of the amino acid residues that define the MAPKAPK2 or MAPKAPK2-like ATP-binding pocket. The structure coordinates of residues that constitute part of a MAPKAPK2 or MAPKAPK2-like ATP-binding pocket may be specific for defining the chemical environment of the binding pocket, or useful in designing fragments of an inhibitor that may interact with those residues. For example, the portion of residues may be key residues that play a role in ligand binding, or may be residues that are spatially related and define a three-dimensional compartment of the binding pocket. In one embodiment, part of the MAPKAPK2 or MAPKAPK2-like ATP-binding pocket is at least two amino acid residues.

The term “MAPKAPK2 protein nuclear export signal motif” refers to the sequence in MAPKAPK2 protein that triggers nuclear export. In MAPKAPK2, the motif is from around residues 345 to 368.

The term “part of a MAPKAPK2 protein nuclear export signal motif” or “part of a MAPKAPK2-like protein nuclear export signal motif” refers to a portion of the MAPKAPK2 or MAPKAPK2-like protein nuclear export signal motif. The structure coordinates of residues that constitute part of a MAPKAPK2 or MAPKAPK2-like protein nuclear export signal motif may be specific for defining the chemical environment of the motif, or useful in designing fragments of an inhibitor that may interact with those residues. For example, the portion of residues may be key residues that play a role in ligand binding, or may be residues that are spatially related and define a three-dimensional compartment of the motif.

The term “MAPKAPK2 protein nuclear localization signal motif” refers to the sequence in the MAPKAPK2 protein that triggers localization of the protein to the cell nucleus. In MAPKAPK2, the motif is from around residues 373 to 389.

The term “part of a MAPKAPK2 protein nuclear localization signal motif” or “part of a MAPKAPK2-like protein nuclear localization signal motif” refers to the portion of the MAPKAPK2 or MAPKAPK2-like protein nuclear localization signal motif. The structure coordinates of residues that constitute part of a MAPKAPK2 or MAPKAPK2-like protein nuclear localization signal motif may be specific for defining the chemical environment of the motif, or useful in designing fragments of an inhibitor that may interact with those residues. For example, the portion of residues may be key residues that are spatially related and define a three-dimensional compartment of the motif.

The term “MAPKAPK2 kinase domain” refers to the catalytic domain at the N-terminus of a MAPKAPK2 protein. The kinase domain includes, for example, the active site that comprises the catalytic residues, the activation loop, the DFG loop, the glycine-rich phosphate anchor loop [See, Xie et al., Structure, 6, pp. 983-991 (1998), incorporated herein by reference] and the αC helix.

The term “part of a MAPKAPK2 kinase domain” or “part of a MAPKAPK2-like kinase domain” refers to a portion of the MAPKAPK2 or MAPKAPK2-like catalytic domain. The structure coordinates of residues that constitute part of a MAPKAPK2 or MAPKAPK2-like kinase domain may be specific for defining the chemical environment of the domain, or useful in designing fragments of an inhibitor that may interact with those residues. For example, the portion of residues may be key residues that play a role in ligand binding, or may be residues that are spatially related and define a three-dimensional compartment of the domain. For example, part of a MAPKAPK2 kinase domain can be the active site, the DFG loop, activation loop, catalytic loop, glycine-rich phosphate anchor loop or the αC helix.

The term “MAPKAPK2 C-terminal regulatory domain” refers to the regulatory domain at the C-terminus of the MAPKAPK2 protein. The domain includes, for example, regulatory phosphorylation sites, the substrate binding pocket, the nuclear localization signal and nuclear export signal.

The term “part of a MAPKAPK2 protein C-terminal regulatory domain” or “part of a MAPKAPK2-like protein C-terminal regulatory domain” is a portion of the residues in the MAPKAPK2 or MAPKAPK2-like C-terminal regulatory domain. The structure coordinates of residues that constitute part of a MAPKAPK2 or MAPKAPK2-like C-terminal regulatory domain may be specific for defining the chemical environment of this domain, or useful in designing fragments of an inhibitor that may interact with those residues. For example, the portion of residues may be key residues that play a role in ligand binding, or may be residues that are spatially related and define a three-dimensional compartment of the domain. For example, part of the domain can be the regulatory phosphorylation sites, the substrate binding pocket, the nuclear localization signal and nuclear export signal.

The term “part of a MAPKAPK2 protein” or “part of a MAPKAPK2 homologue” refers to a portion of the amino acid residues of a MAPKAPK2 protein or homologue that define the binding pockets, domains and motifs. The structure coordinates of residues that constitute part of a MAPKAPK2 protein or homologue may be specific for defining the chemical environment of the protein, or useful in designing fragments of an inhibitor that may interact with those residues. The portion of residues may also be residues that are spatially related and define a three-dimensional compartment of a binding pocket, motif or domain. For example, the portion of residues may be key residues that play a role in ligand or substrate binding, nuclear export, nuclear localization, catalysis, and structural stabilization.

The term “MAPKAPK2 protein complex” or “MAPKAPK2 homologue complex” refers to a molecular complex formed by associating the MAPKAPK2 protein or MAPKAPK2 homologue with a chemical entity, for example, a ligand, a substrate, nucleotide triphosphate, an agonist or antagonist, inhibitor, drug or compound. In one embodiment, the chemical entity is selected from the group consisting of an ATP, a nucleotide triphosphate and an inhibitor for the ATP-binding pocket. In one embodiment, the inhibitor is an ATP analog such as Mg-AMP-PNP (adenylyl-imidodiphosphate).

The term “motif” refers to a portion of the MAPKAPK2 protein or homologue that defines a structural compartment or carries out a function in the protein, for example, catalysis, structural stabilization, phosphorylation, signaling or anchoring. The motif may be conserved in sequence, structure and function when compared to other kinases or related proteins. The motif can be contiguous in primary sequence or three-dimensional space. The motif can comprise α-helices and β-sheets. Examples of a motif include but are not limited to a binding pocket, active site, phosphorylation lip or activation loop, the glycine-rich phosphate anchor loop, the catalytic loop, the DFG loop [See, Xie et al., Structure, 6, pp. 983-991 (1998)), the nuclear localization signal and the nuclear export signal.

The term “root mean square deviation” or “RMSD” means the square root of the arithmetic mean of the squares of the deviations from the mean. It is a way to express the deviation or variation from a trend or object. For purposes of this invention, the “root mean square deviation” defines the variation in the backbone of a protein from the backbone of MAPKAPK2, a binding pocket, a motif, a domain, or portion thereof, as defined by the structure coordinates of MAPKAPK2 described herein.

The term “soaked” refers to a process in which the crystal is transferred to a solution containing the compound of interest.

The term “structure coordinates” refers to Cartesian coordinates derived from mathematical equations related to the patterns obtained on diffraction of a monochromatic beam of X-rays by the atoms (scattering centers) of a protein or protein complex in crystal form. The diffraction data are used to calculate an electron density map of the repeating unit of the crystal. The electron density maps are then used to establish the positions of the individual atoms of the molecule or molecular complex.

The term “substrate binding pocket” refers to the binding pocket for a substrate of MAPKAPK2 or homologue thereof. A substrate is generally defined as the molecule upon which an enzyme performs catalysis. Natural substrates, such as HSP25/HSP27 and glycogen synthase, synthetic substrates or peptides or mimics of natural substrates of MAPKAPK2 or its homologues may associate with the substrate binding pocket.

The term “sufficiently homologous to MAPKAPK2” refers to a protein that has a sequence homology of at least 20% compared to MAPKAPK2 protein. In other embodiments, the sequence homology is at least 40%, at least 60%, at least 80%, at least 90% or at least 95%.

The term “three-dimensional structural information” refers to information taken from the structure coordinates. Structural information generated can include the three-dimensional structure or graphical representation of the structure. Structural information can also be generated when subtracting distances between atoms in the structure coordinates, calculating chemical energies for a MAPKAPK2 molecule or molecular complex or homologues thereof, calculating or minimizing energies for an association of a MAPKAPK2 molecule or molecular complex or homologues thereof to a chemical entity.

Crystallizable Compositions and Crystals of MAPKAPK2 Protein and Protein Complexes

According to one embodiment, the invention provides a crystallizable composition comprising MAPKAPK2 protein and phosphate ions. The MAPKAPK2 protein may be phosphorylated or unphosphorylated.

In one embodiment, the aforementioned crystallizable composition comprises a precipitant, Na/K phosphate at between about 1-3 M, pH at between about 4.0 and 6.0. In one embodiment, the pH is 5.15 and the Na/K phosphate concentration is 2 M. The MAPKAPK2 protein is preferably 85-100% pure prior to forming the composition.

According to another embodiment, the invention provides a crystal composition comprising MAPKAPK2 protein. Preferably, the crystal has a unit cell dimension of a=b=143.994 Å, c=90.273 Å, α=β=90°, γ=120° and belongs to space group P1 or R3. It will be readily apparent to those skilled in the art that the unit cells of the crystal compositions may deviate ±1-2 Å from the above cell dimensions depending on the deviation in the unit cell calculations.

As used herein, the MAPKAPK2 protein in the crystal or crystallizable composition can be the full length protein (amino acids 1-400 as shown in SEQ ID NO: 1); a truncated protein with amino acids 47-400, 47-385 or 47-378 according to SEQ ID NO: 1; or the full length or truncated protein with conservative substitutions.

The MAPKAPK2 protein or a MAPKAPK2-like protein may be produced by any well-known method, including synthetic methods, such as solid phase, liquid phase and combination solid phase/liquid phase syntheses; recombinant DNA methods, including cDNA cloning, optionally combined with site directed mutagenesis; and/or purification of the natural products. In one embodiment, the protein is overexpressed from an E. Coli system.

The invention also relates to a method of making crystals of MAPKAPK2 protein or a homologue thereof. Such methods comprise the steps of:
a) producing and purifying MAPKAPK2 protein or its homologue;
b) mixing said MAPKAPK2 protein or a homologue thereof with a crystallization solution to produce a crystallizable composition; and
c) subjecting said composition to devices and conditions that promote crystallization.

The invention also relates to the method of making crystals wherein step (b) further comprises adding a chemical entity to the MAPKAPK2 protein or homologue thereof.

In one embodiment, the crystallizable composition is made according to the conditions discussed above. In another embodiment, the chemical entity binds to the substrate binding pocket or ATP-binding pocket.

Devices for promoting crystallization can include but are not limited to the hanging-drop, sitting-drop, dialysis or microtube batch devices. [U.S. Pat. Nos. 4,886,646, 5,096,676, 5,130,105, 5,221,410 and 5,400,741; Pav et al., Proteins: Structure, Function, and Genetics, 20, pp. 98-102 (1994), incorporated herein by reference]. The hanging-drop or sitting-drop methods produce crystals by vapor diffusion. The hanging-drop, sitting-drop, and some adaptations of the microbatch methods (D'Arcy et al., J. Cryst. Growth, 168, pp. 175-180 (1996) and Chayen, J. Appl. Cryst., 30, pp. 198-202 (1997)) produce crystals by vapor diffusion. The hanging drop and sitting drop containing the crystallizable composition is equilibrated in a reservoir containing a higher or lower concentration of the precipitant. As the drop approaches equilibrium with the reservoir, the saturation of protein in the solution leads to the formation of crystals.

Microseeding or seeding may be used to obtain larger, or better quality (i.e., crystals with higher resolution diffraction or single crystals) crystals from initial micro-crystals. Microseeding involves the use of crystalline particles to provide nucleation under controlled crystallization conditions. Microseeding is used to increase the size and quality of crystals. In this instance, micro-crystals are crushed to yield a stock seed solution. The stock seed solution is diluted in series. Using a needle, glass rod or strand of hair, a small sample from each diluted solution is added to a set of equilibrated drops containing a protein concentration equal to or less than a concentration needed to create crystals without the presence of seeds. The aim is to end up with a single seed crystal that will act to nucleate crystal growth in the drop.

It would be readily apparent to one of skill in the art following the teachings of the specification to vary the crystallization conditions disclosed herein to identify other crystallization conditions that would produce crystals of MAPKAPK2, MAPKAPK2 protein complex or homologues thereof. Such variations include, but are not limited to, adjusting pH, protein concentration and/or crystallization temperature, changing the identity or concentration of salt and/or precipitant used, using a different method of crystallization, or introducing additives such as detergents (e.g., TWEEN 20 (monolaurate), LDAO, Brij 30 (4 lauryl ether)), sugars (e.g., glucose, maltose), organic compounds (e.g., dioxane, dimethylformamide), lanthanide ions or polyionic compounds that aid in crystallization. High throughput crystallization assays may also be used to assist in finding or optimizing the crystallization conditions.

Binding Pockets of MAPKAPK2 Protein, or Homologues thereof.

As disclosed above, applicants have provided for the first time the three-dimensional X-ray crystal structure of unphosphorylated MAPKAPK2. The crystal structure of MAPKAPK2 presented here is the first reported within the MAPKAP kinase family. The invention will be useful for inhibitor design in treating diseases associated with MAPKAPK2. The atomic coordinate data is presented in FIG. 1.

In order to use the structure coordinates generated for the MAPKAPK2, one of its binding pockets, domains, motifs, or portions thereof, it is often times necessary to convert them into a three-dimensional shape. This is achieved through the use of commercially available software that is capable of generating three-dimensional structures of molecules or portions thereof from a set of structure coordinates.

Binding pockets, also referred to as binding sites in the present invention, are of significant utility in fields such as drug discovery. The association of natural ligands or substrates with the binding pockets of their corresponding receptors or enzymes is the basis of many biological mechanisms of action. Similarly, many drugs exert their biological effects through association with the binding pockets of receptors and enzymes. Such associations may occur with all or part of the binding pocket. An understanding of such associations of MAPKAPK2 and MAPKAPK2 homologues with the molecules that bind to their binding pockets will help lead to the design of drugs having favorable associations with their target receptor or enzyme, and thus, favorable biological effects. Therefore, in one embodiment, this information is valuable in designing potential inhibitors of the binding pockets of biologically important targets. The MAPKAPK2 and MAPKAPK2 homologue ATP and substrate binding pockets of this invention will be important for drug design.

In one embodiment, the ATP-binding pocket consists of amino acids Lys77, Va178, Gln80, Ala91, Leu92, Lys93, Glu104, His108, Val118, Ile136, Met138, Glu139, Cys140, Leu141, Gly144, Glu145, Asp186, Glu190, Asn191, Leu193, Thr206 and Asp207 of FIG. 1. These amino acid residues were identified as illustrated in Example 8. In the MAPKAPK2 ATP-binding pocket, the electron density of the side chains of Lys77 and Val78 could not be located. Therefore, at these positions, Ala residues were used to build the structure model. For the purpose of this invention, the structure coordinates of Lys77 and Val78 refer to the structure coordinates of Ala77 and Ala78 in FIG. 1, respectively. The corresponding amino acids of these residues may be identified by structural alignment using the structure coordinates of Ala77 and Ala78 in FIG. 1 or by sequence alignment using Lys77 and Val78 at these positions.

In another embodiment, the ATP-binding pocket consists of amino acid residues Lys77, Leu92, His108, Ile136, Glu139 and Cys140 of FIG. 1. These residues were found to be conserved between MAPKAPK2, MAPKAPK3 and MAPKAPK4. In another embodiment, the ATP-binding pocket consists of amino acid residues Lys77, Gln80, Leu92, His108, Ile136, Glu139, Cys140 and Leu 141 of FIG. 1. In MAPKAPK2, Gln80 and Leu141 were found to be unique compared to the amino acid residues in MAPKAPK3 and MAPKAPK4 at corresponding positions.

In one embodiment, the substrate binding pocket consists of amino acid residues Gln145, Phe147, Gln151, Phe158, Glu160, Arg161, Ser164, Arg185, Asp186, Lys188, Pro189, Glu190, Phe210, Tyr240, Cys244, Trp247, Ser248, Val251, Ile252, Leu256, Leu257, Gly259, Tyr260 and Pro261 in FIG. 1. These amino acids were identified as illustrated in Example 9. In another embodiment, the substrate binding pocket consists of amino acid residues Gln151, Phe158, Glu160, Arg185, Lys188, Tyr240, Leu256 and Leu257 in FIG. 1. These amino acid residues were found to be in direct contact with the substrate NES.

In one embodiment, the nuclear export signal (NES) motif of MAPKAPK2 consists of amino acid residues Asp345, Lys346, Glu347, Arg348, Trp349, Glu350, Asp351, Val352, Lys353, Glu354, Glu355, Met356, Thr357, Ser358, Ala359, Leu360, Ala361, Thr362, Met363, Arg364, Val365, Asp366, Tyr367 and Glu368 in FIG. 1. In the MAPKAPK2 NES motif, the electron density of the side chains of Glu347, Glu350 and Asp351 could not be located. Therefore, at these positions, Ala residues were used to build the structure model. For the purpose of this invention, the structure coordinates of Glu347, Glu350 and Asp351 refer to the structure coordinates of Ala347, Ala350 and Ala351 in FIG. 1, respectively. The corresponding amino acids of these residues may be identified by structural alignment using the structure coordinates of Ala347, Ala350 and Ala351 in FIG. 1 or by sequence alignment using Glu347, Glu350 and Asp351 at these positions.

In another embodiment, the nuclear export signal motif consists of amino acid residues Met356, Leu360, Met363 and Val365. Amino acid residues Met356, Leu360 and Met363 are hydrophobic and point to one side of the helix in the NES motif. Val365 is also hydrophobic and points to the other side of the helix.

Thus, the ATP-binding pocket, the substrate binding pocket, and the nuclear export signal motif of this invention are defined by the structural coordinates of the above amino acids, as set forth in FIG. 1.

It will be readily apparent to those of skill in the art that the numbering of amino acids in other homologues of MAPKAPK2 may be different than that set forth for MAPKAPK2. Corresponding amino acids in homologues of MAPKAPK2 are easily identified by visual inspection of the amino acid sequences or by using commercially available sequence homology, structural homology or structure superimposition software programs.

Those of skill in the art understand that a set of structure coordinates for a molecule or a molecular-complex or a portion thereof, is a relative set of points that define a shape in three dimensions. Thus, it is possible that an entirely different set of coordinates could define a similar or identical shape. Moreover, slight variations in the individual coordinates will have little effect on overall shape. In terms of binding pockets, these variations would not be expected to significantly alter the nature of ligands that could associate with those pockets.

The variations in coordinates discussed above may be generated because of mathematical manipulations of the MAPKAPK2 structure coordinates. For example, the structure coordinates set forth in FIG. 1 may undergo crystallographic permutations of the structure coordinates, fractionalization of the structure coordinates, integer additions or subtractions to sets of the structure coordinates, inversion of the structure coordinates or any combination of the above.

Alternatively, modifications in the crystal structure due to mutations, additions, substitutions, and/or deletions of amino acids, or other changes in any of the components that make up the crystal could also account for variations in structure coordinates. If such variations are within a certain root mean square deviation as compared to the original coordinates, the resulting three-dimensional shape is considered encompassed by this invention. Thus, for example, a ligand that bound to the binding pocket of MAPKAPK2 would also be expected to bind to another binding pocket whose structure coordinates defined a shape that fell within the acceptable root mean square deviation.

Various computational analyses maybe necessary to determine whether a binding pocket, motif, domain or portion thereof of a molecule or molecular complex is sufficiently similar to the binding pocket, motif, domain or portion thereof of MAPKAPK2. Such analyses may be carried out in well known software applications, such as ProFit [A. C. R. Martin, SciTech Software, ProFit version 1.8, http://www.bioinf.org.uk/software], Swiss-Pdb Viewer [Guex et al., Electrophoresis, 18, pp. 2714-2723 (1997)], the Molecular Similarity application of QUANTA [Accelrys ©2001, 2002] and as described in the accompanying User's Guide, which are incorporated herein by reference.

The above-identified programs, as well as others known to those of skill in the art, permit comparisons between different structures, different conformations of the same structure, and different parts of the same structure. The procedure used in QUANTA (Accelrys ©2001, 2002) and Swiss-Pdb Viewer to compare structures is divided into four steps: 1) loading the structures to be compared; 2) defining the atom equivalences in these structures; 3) performing a fitting operation on the structures; and 4) analyzing the results.

The procedure used in ProFit to compare structures includes the following steps: 1) loading the structures to be compared; 2) specifying selected residues of interest; 3) defining the atom equivalences in the selected residues; 4) performing a fitting operation on the selected residues; and 5) analyzing the results. Each structure in the comparison is identified by a name. One structure is identified as the target (i.e., the fixed structure); all remaining structures are working structures (i.e., moving structures). Since atom equivalency within the above programs is defined by user input, for the purpose of this invention we will define equivalent atoms as protein backbone atoms (N, Ca, C and O) for MAPKAPK2 amino acids and corresponding amino acids in the structures being compared.

The corresponding amino acids may be identified by sequence alignment programs such as the “bestfit” program available from the Genetics Computer Group that uses the local homology algorithm described by Smith and Waterman in Advances in Applied Mathematics 2, 482 (1981), which is incorporated herein by reference. A suitable amino acid sequence alignment will require that the proteins being aligned share minimum percentage of identical amino acids. Generally, a first protein being aligned with a second protein should share in excess of about 35% identical amino acids with the second protein [Hanks et al., Science, 241, 42 (1988); Hanks and Quinn, Methods in Enzymology, 200, 38 (1991)]. The identification of equivalent residues can also be assisted by secondary structure alignment, for example, aligning the α-helices, β-sheets in the structure. The program Swiss-Pdb viewer has its own best fit algorithm that is based on secondary sequence alignment.

When a rigid fitting method is used, the working structure is translated and rotated to obtain an optimum fit with the target structure. The fitting operation uses an algorithm that computes the optimum translation and rotation to be applied to the moving structure, such that the root mean square difference of the fit over the specified pairs of equivalent atom is an absolute minimum. This number, given in angstroms, is reported by the above programs. The Swiss-Pdb Viewer program sets an RMSD cutoff for eliminating pairs of equivalent atoms that have high RMSD values. For programs that calculate an average of the individual RMSD values of the backbone atoms, an RMSD cutoff value can be used to exclude pairs of equivalent atoms with extreme individual RMSD values. In the program ProFit, the RMSD cutoff value can be specified by the user.

For the purpose of this invention, any molecule, molecular complex, binding pocket, motif, domain thereof or portion thereof that is within a root mean square deviation for backbone atoms (N, Cα, C, O) when superimposed on the relevant backbone atoms described by structure coordinates listed in FIG. 1 are encompassed by this invention.

Therefore one embodiment of this invention provides a crystalline molecule or molecular complex comprising an ATP-binding pocket, wherein said binding pocket is defined by structure coordinates of a set of amino acid residues which are identical to MAPKAPK2 amino acid residues Lys77, Leu92, His108, Ile136, Glu139 and Cys140 according to FIG. 1, wherein the root mean square deviation of the backbone atoms between said set of amino acid residues of said molecule or molecular complex and said MAPKAPK2 amino acid residues is not more than about 3.0 Å, 2.5 Å, 2.0 Å, 1.5 Å or 1.0 Å.

A further embodiment of the invention provides an ATP-binding pocket, wherein said set of amino acid residues further comprise amino acid residues which are identical to MAPKAPK2 amino acid residues Gln80 and Leu141 according to FIG. 1, wherein the root mean square deviation of the backbone atoms between said set of amino acid residues of said molecule or molecular complex and said MAPKAPK2 amino acid residues is not more than about 3.0 Å, 2.5 Å, 2.0 Å, 1.5 Å or 1.0 Å.

Another embodiment of this invention provides a crystalline molecule or molecular complex comprising an ATP-binding pocket, wherein said binding pocket is defined by structure coordinates of a set of amino acid residues which are identical to MAPKAPK2 amino acid residues Lys77, Val78, Gln80, Ala91, Leu92, Lys93, Glu104, His108, Val118, Ile136, Met138, Glu139, Cys140, Leu141, Gly144, Glu145, Asp186, Glu190, Asn191, Leu193, Thr206 and Asp207 according to FIG. 1, wherein the root mean square deviation of the backbone atoms between said set of amino acid residues of said molecule or molecular complex and said MAPKAPK2 amino acid residues is not more than about 3.0 Å, 2.5 Å, 2.0 Å, 1.5 Å or 1.0 Å.

Another embodiment of the present invention provides a crystalline molecule or molecular complex comprising a substrate binding pocket, wherein said binding pocket is defined by structure coordinates of a set of amino acid residues which are identical to MAPKAPK2 amino acid residues Gln151, Phe158, Glu160, Arg185, Lys188, Tyr240, Leu256 and Leu257 according to FIG. 1, wherein the root mean square deviation of the backbone atoms between said set of amino acid residues of said molecule or molecular complex and said MAPKAPK2 amino acid residues is not more than about 3.0 Å, 2.5 Å, 2.0 Å, 1.5 Å or 1.0 Å.

Another embodiment of the invention provides the aforementioned binding pocket, wherein said set of amino acid residues further comprise amino acid residues which are identical to MAPKAPK2 amino acid residues Glu145, Phe147, Arg161, Ser164, Asp186, Pro189, Glu190, Phe210, Cys244, Trp247, Ser248, Val251, Ile252, Gly259, Tyr260 and Pro261 according to FIG. 1, wherein the root mean square deviation of the backbone atoms between said set of amino acid residues of said molecule or molecular complex and said MAPKAPK2 amino acid residues is not more than about 3.0 Å, 2.5 Å, 2.0 Å, 1.5 Å or 1.0 Å.

A further embodiment of the invention provides a crystalline molecule or molecular complex comprising a binding pocket, wherein said binding pocket is defined by structure coordinates of a set of amino acid residues comprising at least two amino acid residues which are identical to MAPKAPK2 amino acid residues Lys77, Val78, Gln80, Ala91, Leu92, Lys93, Glu104, His108, Val118, Ile136, Met138, Glu139, Cys140, Leu141, Gly144, Glu145, Phe147, Gln151, Phe158, Glu160, Arg161, Ser164, Arg185, Asp186, Lys188, Pro189, Glu190, Asn191, Leu193, Thr206, and Asp207, Phe210, Tyr240, Cys244, Trp247, Ser248, Val251, Ile252, Leu256, Leu257, Gly259, Tyr260 and Pro261 according to FIG. 1, wherein the root mean square deviation of the backbone atoms between said set of amino acid residues of said molecule or molecular complex and said MAPKAPK2 amino acid residues is not greater than about 0.2 or 0.1 Å.

Another embodiment of the present invention provides a crystalline molecule or molecular complex comprising a nuclear export signal (NES) binding pocket, wherein said binding pocket is defined by structure coordinates of a set of amino acid residues which are identical to MAPKAPK2 amino acid residues Met356, Leu360, Met363 and Val365 according to FIG. 1, wherein the root mean square deviation of the backbone atoms between said set of amino acid residues of said molecule or molecular complex and said MAPKAPK2 amino acid residues is not more than about 3.0 Å, 2.5 Å, 2.0 Å, 1.5 Å or 1.0 Å.

A further embodiment of the invention provides a crystalline molecule or molecular complex wherein the aforementioned set of amino acid residues of Met356, Leu360, Met363 and Val365 further comprise amino acid residues which are identical to MAPKAPK2 amino acid residues Asp345, Lys346, Glu347, Arg348, Trp349, Glu350, Asp351, Val352, Lys353, Glu354, Glu355, Thr357, Ser358, Ala359, Ala361, Thr362, Arg364, Asp366, Tyr367 and Glu368 according to FIG. 1, wherein the root mean square deviation of the backbone atoms between said set of amino acid residues of said molecule or molecular complex and said MAPKAPK2 amino acid residues is not more than about 3.0 Å, 2.5 Å, 2.0 Å, 1.5 Å or 1.0 Å.

Another embodiment of the invention provides a crystalline molecule or molecular complex comprising a domain defined by structure coordinates of a set of amino acid residues which are identical to MAPKAPK2 amino acids 47-320 according to FIG. 1, wherein the root mean square deviation of the backbone atoms between said set of amino acid residues of said molecule or molecular complex and said MAPKAPK2 amino acid residues is not more than about 5.0 Å, 4.0 Å, 3.0 Å, 2.0 Å or 1.0 Å.

A further embodiment of the invention provides a crystalline molecule or molecular complex comprising a domain defined by structure coordinates of a set of amino acid residues which are identical to MAPKAPK2 amino acids 321-400 according to FIG. 1, wherein the root mean square deviation of the backbone atoms between said set of amino acid residues of said molecule or molecular complex and said MAPKAPK2 amino acid residues is not more than about 5.0 Å, 4.0 Å, 3.0 Å, 2.0 Å or 1.0 Å.

One embodiment of the invention provides a crystalline molecule or molecular complex comprising a protein defined by structure coordinates of a set of amino acid residues which are identical to MAPKAPK2 amino acids according to FIG. 1, wherein the root mean square deviation of the backbone atoms between said set of amino acid residues of said molecule or molecular complex and said MAPKAPK2 amino acid residues is not more than about 5.0 Å, 4.0 Å, 3.0 Å, 2.0 Å or 1.0 Å.

Computer Systems

According to another embodiment, this invention provides a machine-readable data storage medium, comprising a data storage material encoded with machine-readable data, wherein said data defines the above-mentioned molecules or molecular complexes. In one embodiment, the data defines the above-mentioned binding pockets by comprising the structure coordinates of said amino acid residues according to FIG. 1. To use the structure coordinates generated for MAPKAPK2 homologues thereof, or one of its binding pockets, it is at times necessary to convert them into a three-dimensional shape or to extract three-dimensional structural information from them. This is achieved through the use of commercially or publicly available software that is capable of generating a three-dimensional structure of molecules or portions thereof from a set of structure coordinates. In one embodiment, the three-dimensional structure may be displayed as a graphical representation.

Therefore, according to another embodiment, this invention provides a machine-readable data storage medium comprising a data storage material encoded with machine readable data. In one embodiment, a machine programmed with instructions for using said data, is capable of generating a three-dimensional structure of any of the molecule or molecular complexes, or binding pockets thereof, that are described herein.

This invention also provides a computer comprising:
a) a machine-readable data storage medium comprising a data storage material encoded with machine-readable data, wherein the data defines any one of the above binding pockets, domains, or protein of the molecule or molecular complex;
b) a working memory for storing instructions for processing said machine-readable data;
c) a central processing unit (CPU) coupled to the working memory and to the machine-readable data storage medium for processing said machine readable data as well as an instruction or set of instructions for generating three-dimensional structural information of said binding pocket, domain or protein; and
d) output hardware coupled to the CPU for outputting three-dimensional structural information of the binding pocket, domain or protein, or information produced by using the three-dimensional structural information of said binding pocket, domain or protein. The output hardware may include monitors, touchscreens, printers, facsimile machines, modems, disk drives, CD-ROMs, etc.

Three-dimensional data generation may be provided by an instruction or set of instructions such as a computer program or commands for generating a three-dimensional structure or graphical representation from structure coordinates, or by subtracting distances between atoms, calculating chemical energies for a MAPKAPK2 molecule or molecular complex or homologues thereof, or calculating or minimizing energies for an association of a MAPKAPK2 molecule or molecular complex or homologues thereof to a chemical entity. The graphical representation can be generated or displayed by commercially available software programs. Examples of software programs include but are not limited to QUANTA [Accelrys ©2001, 2002], O [Jones et al., Acta Crystallogr. A47, pp. 110-119 (1991)] and RIBBONS [Carson, J. Appl. Crystallogr., 24, pp. 9589-961 (1991)], which are incorporated herein by reference. Certain software programs may imbue this representation with physico-chemical attributes which are known from the chemical composition of the molecule, such as residue charge, hydrophobicity, torsional and rotational degrees of freedom for the residue or segment, etc. Examples of software programs for calculating chemical energies are described in the Rational Drug Design section.

Information about said binding pocket or information produced by using said binding pocket can be outputted through a display terminals, touchscreens, printers, modems, facsimile machines, CD-ROMs or disk drives. The information can be in graphical or alphanumeric form.

FIG. 9 demonstrates one version of these embodiments. System 10 includes a computer 11 comprising a central processing unit (“CPU”) 20, a working memory 22 which may be, e.g., RAM (random-access memory) or “core” memory, mass storage memory 24 (such as one or more disk drives or CD-ROM drives), one or more cathode-ray tube (“CRT”) display terminals 26, one or more keyboards 28, one or more input lines 30, and one or more output lines 40, all of which are interconnected by a conventional bi-directional system bus 50.

Input hardware 35, coupled to computer 11 by input lines 30, may be implemented in a variety of ways. Machine-readable data of this invention may be inputted via the use of a modem or modems 32 connected by a telephone line or dedicated data line 34. Alternatively or additionally, the input hardware 35 may comprise CD-ROM drives or disk drives 24. In conjunction with display terminal 26, keyboard 28 may also be used as an input device.

Output hardware 46, coupled to computer 11 by output lines 40, may similarly be implemented by conventional devices. By way of example, output hardware 46 may include CRT display terminal 26 for displaying a graphical representation of a binding pocket of this invention using a program such as QUANTA as described herein. Output hardware might also include a printer 42, so that hard copy output may be produced, or a disk drive 24, to store system output for later use. Output hardware may also include a display terminal, a CD or DVD recorder, ZIP™ or JAZ™ drive, or other machine-readable data storage device.

In operation, CPU 20 coordinates the use of the various input and output devices 35, 46, coordinates data accesses from mass storage 24 and accesses to and from working memory 22, and determines the sequence of data processing steps. A number of programs may be used to process the machine-readable data of this invention. Such programs are discussed in reference to the computational methods of drug discovery as described herein. Specific references to components of the hardware system 10 are included as appropriate throughout the following description of the data storage medium.

FIG. 10 shows a cross section of a magnetic data storage medium 100 which can be encoded with a machine-readable data that can be carried out by a system such as system 10 of FIG. 9. Medium 100 can be a conventional floppy diskette or hard disk, having a suitable substrate 101, which may be conventional, and a suitable coating 102, which may be conventional, on one or both sides, containing magnetic domains (not visible) whose polarity or orientation can be altered magnetically. Medium 100 may also have an opening (not shown) for receiving the spindle of a disk drive or other data storage device 24.

The magnetic domains of coating 102 of medium 100 are polarized or oriented so as to encode in manner that may be conventional, machine readable data such as that described herein, for execution by a system such as system 10 of FIG. 9.

FIG. 11 shows a cross section of an optically-readable data storage medium 110 which also can be encoded with such a machine-readable data, or set of instructions, which can be carried out by a system such as system 10 of FIG. 9. Medium 110 can be a conventional compact disk read only memory (CD-ROM) or a rewritable medium such as a magneto-optical disk that is optically readable and magneto-optically writable. Medium 100 preferably has a suitable substrate 111, which may be conventional, and a suitable coating 112, which may be conventional, usually of one side of substrate 111.

In the case of CD-ROM, as is well known, coating 112 is reflective and is impressed with a plurality of pits 113 to encode the machine-readable data. The arrangement of pits is read by reflecting laser light off the surface of coating 112. A protective coating 114, which preferably is substantially transparent, is provided on top of coating 112.

In the case of a magneto-optical disk, as is well known, coating 112 has no pits 113, but has a plurality of magnetic domains whose polarity or orientation can be changed magnetically when heated above a certain temperature, as by a laser (not shown). The orientation of the domains can be read by measuring the polarization of laser light reflected from coating 112. The arrangement of the domains encodes the data as described above.

Thus, in accordance with the present invention, data capable of generating the three dimensional structure of the above molecules or molecular complexes, or binding pockets thereof, can be stored in a machine-readable storage medium, which is capable of displaying structural information or a graphical three-dimensional representation of the structure.

Rational Drug Design

The MAPKAPK2 structure coordinates or the three-dimensional graphical representation generated from these coordinates may be used in conjunction with a computer for a variety of purposes, including drug discovery.

For example, the structure encoded by the data may be computationally evaluated for its ability to associate with chemical entities. Chemical entities that associate with MAPKAPK2 may inhibit or activate MAPKAPK2 or its homologues, and are potential drug candidates. Alternatively, the structure encoded by the data may be displayed in a graphical three-dimensional representation on a computer screen. This allows visual inspection of the structure, as well as visual inspection of the structure's association with chemical entities.

Thus, according to another embodiment, the invention provides a method for designing, selecting and/or optimizing a chemical entity that binds to the molecule or molecular complex comprising the steps of:
(a) providing the structure coordinates of said molecule or molecular complex on a computer comprising the means for generating three-dimensional structural information from said structure coordinates; and
(b) designing, selecting and/or optimizing said chemical entity by employing means for performing a fitting operation between said compound and said three-dimensional structural information of said molecular complex.

Three-dimensional structural information in step (a) may be generated by instructions such as a computer program or commands that can generate a three-dimensional structure or graphical representation; subtract distances between atoms; calculate chemical energies for a MAPKAPK2 molecule, molecular complex or homologues thereof; or calculate or minimize energies of an association of MAPKAPK2 molecule, molecular complex or homologues thereof to a chemical entity. These types of computer programs are known in the art. The graphical representation can be generated or displayed by commercially available software programs. Examples of software programs include but are not limited to QUANTA [Accelrys ©2001, 2002], O [Jones et al., Acta Crystallogr. A47, pp. 110-119 (1991)] and RIBBONS (Carson, J. Appl. Crystallogr., 24, pp. 9589-961 (1991)], which are incorporated herein by reference. Certain software programs may imbue this representation with physico-chemical attributes which are known from the chemical composition of the molecule, such as residue charge, hydrophobicity, torsional and rotational degrees of freedom for the residue or segment, etc. Examples of software programs for calculating chemical energies are described herein below.

Another embodiment of the invention provides a method for evaluating the potential of a chemical entity to associate with the molecule or molecular complex as described previously.

This method comprises the steps of: a) employing computational means to perform a fitting operation between the chemical entity and the molecule or molecular complex described before; b) analyzing the results of said fitting operation to quantify the association between the chemical entity and the molecule or molecular complex; and, optionally, c) outputting said quantified association to a suitable output hardware, such as a CRT display terminal, a CD or DVD recorder, ZIP™ or JAZ™ drive, a disk drive, or other machine-readable data storage device, as described previously. The method may further comprise generating a three-dimensional structure, graphical representation thereof, or both, of the molecule or molecular complex prior to step a). In one embodiment, the method is for evaluating the ability of a chemical entity to associate with the binding pocket of a molecule or molecular complex.

In another embodiment, the method comprises the steps of:
a) constructing a computer model of a binding pocket of the molecule or molecular complex;
b) selecting a chemical entity to be evaluated by a method selected from the group consisting of assembling said chemical entity; selecting a chemical entity from a small molecule database; de novo ligand design of said chemical entity; and modifying a known agonist or inhibitor, or a portion thereof, of a MAPKAPK2 protein or homologue thereof;
c) employing computational means to perform a fitting program operation between computer models of said chemical entity to be evaluated and said binding pocket in order to provide an energy-minimized configuration of said chemical entity in the binding pocket; and
d) evaluating the results of said fitting operation to quantify the association between said chemical entity and the binding pocket model, whereby evaluating the ability of said chemical entity to associate with said binding pocket.

In another embodiment, the invention provides a method of using a computer for evaluating the ability of a chemical entity to associate with the molecule or molecular complex, wherein said computer comprises a machine-readable data storage medium comprising a data storage material encoded with said structure coordinates defining said binding pocket and means for generating a three-dimensional graphical representation of the binding pocket, and wherein said method comprises the steps of:
(a) positioning said chemical entity within all or part of said binding pocket using a graphical three-dimensional representation of the structure of the chemical entity and the binding pocket;
(b) performing a fitting operation between said chemical entity and said binding pocket by employing computational means;
(c) analyzing the results of said fitting operation to quantitate the association between said chemical entity and all or part of the binding pocket; and
(d) outputting said quantitated association to suitable output hardware.

The above method may further comprise the steps of:
(e) repeating steps (a) through (d) with another chemical entity; and
(f) selecting at least one of said plurality of chemical entities that associates with said all or part of said binding pocket based on said quantitated association of said chemical entity.

Alternatively, the structure coordinates of the MAPKAPK2 binding pockets may be utilized in a method for identifying an agonist or antagonist of a molecule comprising a binding pocket of MAPKAPK2. This method comprises the steps of:
a) using a three-dimensional structure of the molecule or molecular complex to design, select or optimize a chemical entity;
b) contacting the chemical entity with the molecule or molecular complex, and monitoring the activity of the molecule or molecular complex;
c) monitoring the catalytic activity of the molecule or molecular complex; and
d) classifying the chemical entity as an agonist or antagonist based on the effect of the chemical entity on the activity of the molecule or molecular complex.

In one embodiment, step a) is performed using a three-dimensional structure of the binding pocket or portion thereof of the molecule or molecular complex. In another embodiment, the three-dimensional structure is displayed as a graphical representation.

In another embodiment, the method comprises the steps of:
a) constructing a computer model of a binding pocket of the molecule or molecular complex;
b) selecting a chemical entity to be evaluated by a method selected from the group consisting of assembling said chemical entity; selecting a chemical entity from a small molecule database; de novo ligand design of said chemical entity; and modifying a known agonist or inhibitor, or a portion thereof, of a MAPKAPK2 protein or homologue thereof;
c) employing computational means to perform a fitting program operation between computer models of said chemical entity to be evaluated and said binding pocket in order to provide an energy-minimized configuration of said chemical entity in the binding pocket; and
d) evaluating the results of said fitting operation to quantify the association between said chemical entity and the binding pocket model, whereby evaluating the ability of said chemical entity to associate with said binding pocket;
e) synthesizing said chemical entity; and
f) contacting said chemical entity with said molecule or molecular complex to determine the ability of said compound to activate or inhibit said molecule.

For the first time, the present invention permits the use of molecular design techniques to identify, select and design chemical entities, including inhibitory compounds, capable of binding to MAPKAPK2 or MAPKAP-2 like binding pockets, motifs and domains.

Applicants' elucidation of binding pockets on MAPKAPK2 provides the necessary information for designing new chemical entities and compounds that may interact with MAPKAPK2 or MAPKAPK2-like substrate or ATP-binding pockets, in whole or in part. Due to the homology in the kinase core between MAPKAPK2, MAPKAPK3 and MAPKAPK4, compounds that inhibit MAPKAPK2 are also expected to inhibit MAPKAPK3 and MAPKAPK4, especially those compounds that bind the ATP-binding pocket.

Throughout this section, discussions about the ability of an entity to bind to, associate with or inhibit MAPKAPK2 binding pockets refers to features of the entity alone. Assays to determine if a compound binds to MAPKAPK2 are well known in the art and are exemplified below.

The design of compounds that bind to or inhibit MAPKAPK2 binding pockets according to this invention generally involves consideration of two factors. First, the entity must be capable of physically and structurally associating with parts or all of the MAPKAPK2 binding pockets. Non-covalent molecular interactions important in this association include hydrogen bonding, van der Waals interactions, hydrophobic interactions and electrostatic interactions.

Second, the entity must be able to assume a conformation that allows it to associate with the MAPKAPK2 binding pockets directly. Although certain portions of the entity will not directly participate in these associations, those portions of the entity may still influence the overall conformation of the molecule. This, in turn, may have a significant impact on potency. Such conformational requirements include the overall three-dimensional structure and orientation of the chemical entity in relation to all or a portion of the binding pocket, or the spacing between functional groups of an entity comprising several chemical entities that directly interact with the MAPKAPK2 or MAPKAPK2-like binding pockets.

The potential inhibitory or binding effect of a chemical entity on MAPKAPK2 binding pockets may be analyzed prior to its actual synthesis and testing by the use of computer modeling techniques. If the theoretical structure of the given entity suggests insufficient interaction and association between it and the MAPKAPK2 binding pockets, testing of the entity is obviated. However, if computer modeling indicates a strong interaction, the molecule may then be synthesized and tested for its ability to bind to a MAPKAPK2 binding pocket. This may be achieved by testing the ability of the molecule to inhibit MAPKAPK2 using the assays described in Example 7. In this manner, synthesis of inoperative compounds may be avoided.

A potential inhibitor of a MAPKAPK2 binding pocket may be computationally evaluated by means of a series of steps in which chemical entities or fragments are screened and selected for their ability to associate with the MAPKAPK2 binding pockets.

One skilled in the art may use one of several methods to screen chemical entities or fragments for their ability to associate with a MAPKAPK2 binding pocket. This process may begin by visual inspection of, for example, a MAPKAPK2 binding pocket on the computer screen based on the MAPKAPK2 structure coordinates in FIG. 1 or other coordinates which define a similar shape generated from the machine-readable storage medium. Selected fragments or chemical entities may then be positioned in a variety of orientations, or docked, within that binding pocket as defined supra. Docking may be accomplished using software such as QUANTA and Sybyl, followed by energy minimization and molecular dynamics with standard molecular mechanics force fields, such as CHARMM and AMBER.

Specialized computer programs may also assist in the process of selecting fragments or chemical entities. These include:

1. GRID [P. J. Goodford, “A Computational Procedure for Determining Energetically Favorable Binding Sites on Biologically Important Macromolecules”, J. Med. Chem., 28, pp. 849-857 (1985)]. GRID is available from Oxford University, Oxford, UK.

2. MCSS [A. Miranker et al., “Functionality Maps of Binding Sites: A Multiple Copy Simultaneous Search Method.” Proteins: Structure, Function and Genetics, 11, pp. 29-34 (1991)]. MCSS is available from Molecular Simulations, San Diego, Calif.

3. AUTODOCK [D. S. Goodsell et al., “Automated Docking of Substrates to Proteins by Simulated Annealing”, Proteins: Structure, Function, and Genetics, 8, pp. 195-202 (1990)]. AUTODOCK is available from Scripps Research Institute, La Jolla, Calif.

4. DOCK [I. D. Kuntz et al., “A Geometric Approach to Macromolecule-Ligand Interactions”, J. Mol. Biol., 161, pp. 269-288 (1982)]. DOCK is available from University of California, San Francisco, Calif.

Once suitable chemical entities or fragments have been selected, they can be assembled into a single compound or complex. Assembly may be preceded by visual inspection of the relationship of the fragments to each other on the three-dimensional image displayed on a computer screen in relation to the structure coordinates of MAPKAPK2. This would be followed by manual model building using software such as QUANTA or Sybyl [Tripos Associates, St. Louis, Mo.].

Useful programs to aid one of skill in the art in connecting the individual chemical entities or fragments include:

1. CAVEAT [P. A. Bartlett et al., “CAVEAT: A Program to Facilitate the Structure-Derived Design of Biologically Active Molecules”, in Molecular Recognition in Chemical and Biological Problems”, Special Pub., Royal Chem. Soc., 78, pp. 182-196 (1989); G. Lauri and P. A. Bartlett, “CAVEAT: a Program to Facilitate the Design of Organic Molecules”, J. Comput. Aided Mol. Des., 8, pp. 51-66 (1994)]. CAVEAT is available from the University of California, Berkeley, Calif.

2. 3D Database systems such as ISIS (MDL Information Systems, San Leandro, Calif.). This area is reviewed in Y. C. Martin, “3D Database Searching in Drug Design”, J. Med. Chem., 35, pp. 2145-2154 (1992).

3. HOOK [M. B. Eisen et al., “HOOK: A Program for Finding Novel Molecular Architectures that Satisfy the Chemical and Steric Requirements of a Macromolecule Binding Site”, Proteins: Struct., Funct., Genet., 19, pp. 199-221 (1994)]. HOOK is available from Molecular Simulations, San Diego, Calif.

Instead of proceeding to build an inhibitor of a MAPKAPK2 binding pocket in a step-wise fashion one fragment or chemical entity at a time as described above, inhibitory or other MAPKAPK2 binding compounds may be designed as a whole or “de novo” using either an empty binding pocket or optionally including some portion(s) of a known inhibitor(s). There are many de novo ligand design methods including:

1. LUDI [H. -J. Bohm, “The Computer Program LUDI: A New Method for the De Novo Design of Enzyme Inhibitors”, J. Comp. Aid. Molec. Design, 6, pp. 61-78 (1992)]. LUDI is available from Molecular Simulations Incorporated, San Diego, Calif.

2. LEGEND [Y. Nishibata et al., Tetrahedron, 47, p. 8985 (1991)]. LEGEND is available from Molecular Simulations Incorporated, San Diego, Calif.

3. LeapFrog [available from Tripos Associates, St. Louis, Mo.].

4. SPROUT [V. Gillet et al., “SPROUT: A Program for Structure Generation)”, J. Comput. Aided Mol. Design, 7, pp. 127-153 (1993)]. SPROUT is available from the University of Leeds, UK.

Other molecular modeling techniques may also be employed in accordance with this invention [see, e.g., N. C. Cohen et al., “Molecular Modeling Software and Methods for Medicinal Chemistry, J. Med. Chem., 33, pp. 883-894 (1990); see also, M. A. Navia and M. A. Murcko, “The Use of Structural Information in Drug Design”, Current Opinions in Structural Biology, 2, pp. 202-210 (1992); L. M. Balbes et al., “A Perspective of Modern Methods in Computer-Aided Drug Design”, Reviews in Computational Chemistry, Vol. 5, K. B. Lipkowitz and D. B. Boyd, Eds., VCH, New York, pp. 337-380 (1994); see also, W. C. Guida, “Software For Structure-Based Drug Design”, Curr. Opin. Struct. Biology, 4, pp. 777-781 (1994)].

Once a compound has been designed or selected by the above methods, the efficiency with which that entity may bind to a MAPKAPK2 binding pocket may be tested and optimized by computational evaluation. For example, an effective MAPKAPK2 binding pocket inhibitor must preferably demonstrate a relatively small difference in energy between its bound and free states (i.e., a small deformation energy of binding). Thus, the most efficient MAPKAPK2 binding pocket inhibitors should preferably be designed with a deformation energy of binding of not greater than about 10 kcal/mole, more preferably, not greater than 7 kcal/mole. MAPKAPK2 binding pocket inhibitors may interact with the binding pocket in more than one conformation that is similar in overall binding energy. In those cases, the deformation energy of binding is taken to be the difference between the energy of the free entity and the average energy of the conformations observed when the inhibitor binds to the protein.

An entity designed or selected as binding to a MAPKAPK2 binding pocket may be further computationally optimized so that in its bound state it would preferably lack repulsive electrostatic interaction with the target enzyme and with the surrounding water molecules. Such non-complementary electrostatic interactions include repulsive charge-charge, dipole-dipole and charge-dipole interactions.

Specific computer software is available in the art to evaluate compound deformation energy and electrostatic interactions. Examples of programs designed for such uses include: Gaussian 94, revision C [M. J. Frisch, Gaussian, Inc., Pittsburgh, Pa. ©1995]; AMBER, version 4.1 [P. A. Kollman, University of California at San Francisco, ©1995]; QUANTA/CHARMM [Accelrys, San Diego, Calif. ©2001, 2002]; Insight II/Discover [Accelrys, San Diego, Calif. ©2001, 2002]; DelPhi (Accelrys, San Diego, Calif. ©2001, 2002]; and AMSOL [Quantum Chemistry Program Exchange, Indiana University]. These programs may be implemented, for instance, using a Silicon Graphics workstation such as an Indigo2 with “IMPACT” graphics. Other hardware systems and software packages will be known to those skilled in the art.

Another approach enabled by this invention, is the computational screening of small molecule databases for chemical entities or compounds that can bind in whole, or in part, to a MAPKAPK2 binding pocket. In this screening, the quality of fit of such entities to the binding pocket may be judged either by shape complementarity or by estimated interaction energy [E. C. Meng et al., J. Comp. Chem., 13, pp. 505-524 (1992)].

Another particularly useful drug design technique enabled by this invention is iterative drug design. Iterative drug design is a method for optimizing associations between a protein and a compound by determining and evaluating the three-dimensional structures of successive sets of protein/compound complexes.

In iterative drug design, crystals of a series of protein or protein complexes are obtained and then the three-dimensional structures of each crystal is solved. Such an approach provides insight into the association between the proteins and compounds of each complex. This is accomplished by selecting compounds with inhibitory activity, obtaining crystals of this new protein/compound complex, solving the three-dimensional structure of the complex, and comparing the associations between the new protein/compound complex and previously solved protein/compound complexes. By observing how changes in the compound affected the protein/compound associations, these associations may be optimized.

In some cases, iterative drug design is carried out by forming successive protein-compound complexes and then crystallizing each new complex. Alternatively, a pre-formed protein crystal is soaked in the presence of an inhibitor, thereby forming a protein/compound complex and obviating the need to crystallize each individual protein/compound complex.

Structure Determination of Other Molecules

The structure coordinates set forth in FIG. 1 can also be used to aid in obtaining structural information about another crystallized molecule or molecular complex. This may be achieved by any of a number of well-known techniques, including molecular replacement.

In one embodiment, the structure coordinates of said molecules or molecular complexes are produced by homology modeling of the coordinates of FIG. 1. Homology modeling can be used to generate structural models of MAPKAPK2 homologues or other homologous proteins based on the known structure of MAPKAPK2. This can be achieved by performing one or more of the following steps: performing sequence alignment between the amino acid sequence of an unknown molecule against the amino acid of MAPKAPK2; identifying conserved and variable regions by sequence or structure; generating structure co-ordinates for structurally conserved residues of the unknown structure from those of MAPKAPK2; generating conformations for the structurally variable residues in the unknown structure; replacing the non-conserved residues of MAPKAPK2 with residues in the unknown structure; building side chain conformations; and refining and/or evaluating the unknown structure.

For example, since the protein sequence of the catalytic domains of MAPKAPK2 and MAPKAPK3 can be aligned relative to each other, it is possible to construct models of the structures of MAPKAPK3, particularly in the regions of the active site, using the MAPKAPK2 structure. Software programs that are useful in homology modeling include XALIGN [Wishart, D. S. et al., Comput. Appl. Biosci., 10, pp. 687-88 (1994)] and CLUSTAL W Alignment Tool [Higgins D. G. et al., Methods Enzymol, 266, pp. 383-402 (1996)]. See also, U.S. Pat. No. 5,884,230. These references are incorporated herein by reference.

To perform the sequence alignment, programs such as the “bestfit” program available from the Genetics Computer Group [Waterman in Advances in Applied Mathematics 2, 482 (1981), which is incorporated herein by reference] and CLUSTAL W Alignment Tool [Higgins D. G. et al., Methods Enzymol, 266, pp. 383-402 (1996), which is incorporated by reference] can be used. To model the amino acid side chains of MAPKAPK3, the amino acids in MAPKAPK2 can be replaced, using a computer graphics program such as “O” [Jones et al, (1991) Acta Cryst. Sect. A, 47: 110-119], by those of the homologous protein, where they differ. The same orientation or a different orientation of the amino acid can be used. Insertions and deletions of amino acid residues may be necessary where gaps occur in the sequence alignment. However, certain portions of the active site of MAPKAPK2 and its homologues are highly conserved with essentially no insertions and deletions.

Homology modeling can be performed using, for example, the computer programs SWISS-MODEL available through Glaxo Wellcome Experimental Research in Geneva, Switzerland; WHATIF available on EMBL servers; Schnare et al. (1996) J. Mol. Biol, 256: 701-719; Blundell et al. (1987) Nature 326: 347-352; Fetrow and Bryant (1993) Bio/Technology 11:479-484; Greer (1991) Methods in Enzymology 202: 239-252; and Johnson et al (1994) Crit. Rev. Biochem. Mol. Biol. 29:1-68. An example of homology modeling can be found, for example, in Szklarz G. D (1997) Life Sci. 61: 2507-2520. These references are incorporated herein by reference.

According to an alternate embodiment, the machine-readable data storage medium comprises a data storage material encoded with a first set of machine readable data which comprises the Fourier transform of at least a portion of the structure coordinates set forth in FIG. 1, and which, when using a machine programmed with instructions for using said data, can be combined with a second set of machine readable data comprising the X-ray diffraction pattern of a molecule or molecular complex to determine at least a portion of the structure coordinates corresponding to the second set of machine readable data.

In another embodiment, the invention provides a computer for determining at least a portion of the structure coordinates corresponding to X-ray diffraction data obtained from a molecule or molecular complex, wherein said computer comprises:
a) a machine-readable data storage medium comprising a data storage material encoded with machine-readable data, wherein said data comprises at least a portion of the structural coordinates of MAPKAPK2 according to FIG. 1;
b) a machine-readable data storage medium comprising a data storage material encoded with machine-readable data, wherein said data comprises X-ray diffraction data obtained from said molecule or molecular complex; and
c) instructions for performing a Fourier transform of the machine readable data of (a) and for processing said machine readable data of (b) into structure coordinates.

For example, the Fourier transform of at least a portion of the structure coordinates set forth in FIG. 1 may be used to determine at least a portion of the structure coordinates of MAPKAPK2 homologues.

Therefore, in another embodiment this invention provides a method of utilizing molecular replacement to obtain structural information about a molecule or molecular complex whose structure is unknown comprising the steps of:
a) crystallizing said molecule or molecular complex of unknown structure;
b) generating an X-ray diffraction pattern from said crystallized molecule or molecular complex; and
c) applying at least a portion of the structure coordinates set forth in FIG. 1 to the X-ray diffraction pattern to generate a three-dimensional electron density map of the molecule or molecular complex whose structure is unknown; and
d) generating a structural model of the molecule or molecular complex from the three-dimensional electron density map.

In one embodiment, the method is performed using a computer. In another embodiment, the molecule is selected from the group consisting of MAPKAPK2 and MAPKAPK2 homologues. In another embodiment, the molecule is a MAPKAPK2 molecular complex or homologue thereof.

By using molecular replacement, all or part of the structure coordinates of the MAPKAPK2 as provided by this invention (and set forth in FIG. 1) can be used to determine the structure of a crystallized molecule or molecular complex whose structure is unknown more quickly and efficiently than attempting to determine such information ab initio.

Molecular replacement provides an accurate estimation of the phases for an unknown structure. Phases are a factor in equations used to solve crystal structures that can not be determined directly. Obtaining accurate values for the phases, by methods other than molecular replacement, is a time-consuming process that involves iterative cycles of approximations and refinements and greatly hinders the solution of crystal structures. However, when the crystal structure of a protein containing at least a homologous portion has been solved, the phases from the known structure provide a satisfactory estimate of the phases for the unknown structure.

Thus, this method involves generating a preliminary model of a molecule or molecular complex whose structure coordinates are unknown, by orienting and positioning the relevant portion of the MAPKAPK2 according to FIG. 1 within the unit cell of the crystal of the unknown molecule or molecular complex so as best to account for the observed X-ray diffraction pattern of the crystal of the molecule or molecular complex whose structure is unknown. Phases can then be calculated from this model and combined with the observed X-ray diffraction pattern amplitudes to generate an electron density map of the structure whose coordinates are unknown. This, in turn, can be subjected to any well-known model building and structure refinement techniques to provide a final, accurate structure of the unknown crystallized molecule or molecular complex [E. Lattman, “Use of the Rotation and Translation Functions”, in Meth. Enzymol., 115, pp. 55-77 (1985); M. G. Rossmann, ed., “The Molecular Replacement Method”, Int. Sci. Rev. Ser., No. 13, Gordon & Breach, New York (1972)].

The structure of any portion of any crystallized molecule or molecular complex that is sufficiently homologous to any portion of the MAPKAPK2 can be resolved by this method.

In a preferred embodiment, the method of molecular replacement is utilized to obtain structural information about a MAPKAPK2 homologue. The structure coordinates of MAPKAPK2 as provided by this invention are particularly useful in solving the structure of MAPKAPK2 complexes that are bound by ligands, substrates and inhibitors.

Furthermore, the structure coordinates of MAPKAPK2 as provided by this invention are useful in solving the structure of MAPKAPK2 proteins that have amino acid substitutions, additions and/or deletions (referred to collectively as “MAPKAPK2 mutants”, as compared to naturally occurring MAPKAPK2). These MAPKAPK2 mutants may optionally be crystallized in co-complex with a chemical entity, such as a non-hydrolyzable ATP analog or a suicide substrate. The crystal structures of a series of such complexes may then be solved by molecular replacement and compared with that of wild-type MAPKAPK2. Potential sites for modification within the various binding pockets of the enzyme may thus be identified. This information provides an additional tool for determining the most efficient binding interactions, for example, increased hydrophobic interactions, between MAPKAPK2 and a chemical entity or compound.

The structure coordinates are also particularly useful in solving the structure of crystals of MAPKAPK2 or MAPKAPK2 homologues co-complexed with a variety of chemical entities. This approach enables the determination of the optimal sites for interaction between chemical entities, including candidate MAPKAPK2 inhibitors. For example, high resolution X-ray diffraction data collected from crystals exposed to different types of solvent allows the determination of where each type of solvent molecule resides. Small molecules that bind tightly to those sites can then be designed and synthesized and tested for their MAPKAPK2 inhibition activity.

All of the complexes referred to above may be studied using well-known X-ray diffraction techniques and may be refined versus 1.2-3.4 Å resolution X-ray data to an R value of about 0.30 or less using computer software, such as X-PLOR [Yale University, ©1992, distributed by Molecular Simulations, Inc.; see, e.g., Blundell & Johnson, supra; Meth. Enzymol., vol. 114 & 115, H. W. Wyckoff et al., eds., Academic Press (1985)] or CNS [Brunger et al., Acta Crystallogr. D. Biol. Crystallogr., 54, pp. 905-921, (1998)]. This information may thus be used to optimize known MAPKAPK2 inhibitors, and more importantly, to design new MAPKAPK2 inhibitors.

In order that this invention be more fully understood, the following examples are set forth. These examples are for the purpose of illustration only and are not to be construed as limiting the scope of the invention in any way.

### EXAMPLE 1

### Expression of MAPKAPK2

The expression of human MAPKAPK2 was carried out using standard procedures known in the art. Specifically, MAPKAPK2 residues 47-400 were cloned into pBEV1, a T7 polymerase based E. coli expression vector. BL21(DE3) competent cells were then transformed with pBEV1/(HIS)₆-tag (SEQ ID NO: 5) MAPKAPK2(47-400) via a standard transformation protocol.

The freshly transformed cells were grown at 37° C., for 16 hours, in a complex media supplemented with 100 μg/ml carbenicillin. This culture was used to inoculate additional flasks of M9/carbenicillin(1:10). These cultures were then grown to OD₆₀₀ 0.7-0.9, whereupon amino acids lysine, phenylalanine, and threonine were added to final concentrations of 100 mg/L; amino acids seleno-methionine, isoleucine, and valine were added to final concentrations of 50 mg/L. The growth temperature was then reduced to 30° C. After 30 minutes, induction was initiated by the addition of 1 mM IPTG. The cells were then harvested via centrifugation, approximately 14 hours post induction and flash frozen at −80° C. prior to purification.

### EXAMPLE 2

### Purification of MAPKAPK2

The frozen cell paste from Example 1 was thawed in 10 volumes of Buffer A (50 mM HEPES, pH 7.8, 10% glycerol, 2 mM β-mercaptoethanol, 200 mM NaCl, 0.02% Tween 20)+0.5 mM Pefabloc, 2 μg/ml pepstatin, 1 g/ml E64, 1 μg/ml leupeptin and lysed in a microfluidizer. The lysate was centrifuged at 54,000 g for 1 hour. The supernatant was collected and incubated batchwise with Talon metal affinity resin. After extensive washing with Buffer A, the resin was eluted with Buffer A+150 mM imidazole. One unit of thrombin per mg of His-tagged protein was added to the Talon elute pool and allowed to incubate at room temperature for 1 hour. The thrombin activity was quenched by addition of 0.5 mM pefabloc. The protein was diluted 1:4 to lower the NaCl to 50 mM, and loaded onto a Q-sepharose column pre-equilibrated with Buffer A. The flow-through fractions, containing MAPKAPK2, were collected and directly loaded to a SP-sepharose column pre-equilibrated with Buffer B (25 mM HEPES, pH 7.2, 5% glycerol, 2 mM DTT, 0.5 mM pefabloc). The eluted protein from SP-sepharose column was concentrated in a Centroprep-30 for size exclusion chromatography on a Sepherocryl S-200 column pre-equilibrated with Buffer C (25 mM Tris, pH 7.8, 200 mM NaCl, 2 mM DTT). The peak fractions were collected and concentrated to 5-10 mg/ml for crystallization.

### EXAMPLE 3

### Crystallization of MAPKAPK2

Crystals grew by equilibrating a drop containing 10 mg/ml protein solution and equal volume of reservoir solution (2 M of Na/K phosphate at pH 5.15) against the reservoir. Larger crystals were obtained by multi-step seeding, as small seeding crystals were transferred into drops containing protein and precipitant. Most crystals could only be processed in P1 space group with six molecules in an asymmetrical unit. One crystal, which was soaked in Methyl mercury nitrate for overnight was of the space group R3. Once the crystals were harvested, they were transferred to reservoir solutions containing increasing concentrations of glycerol, starting with 5% and increasing to 10, 15, 20, 25 and 30%. After soaking the crystals in 30% glycerol for less than 5 minutes, the crystals were scooped up with a cryo-loop, frozen in liquid nitrogen and stored for data collection.

### EXAMPLE 4

### X-Ray Data Collection and Structure Determination

Data were collected at beam line 5.0.2 of the Advanced Light Source Lawrence Berkeley Laboratory, Berkeley, California using an ADSC Quantum-4 detector. Data were integrated using MOSFLM [A. G. Leslie, Acta Crystallogr. D Biol. Crystallogr., 55, pp. 1696-1702 (1999)] and scaled using SCALA of CCP4 package [(Collaborative Computational Project, N., Acta Cryst., D50, pp. 760-763 (1994)].

The data statistics of the unphosphorylated MAPKAPK2 are summarized in Table 1. The spacegroup of the unphosphorylated MAPKAPK2 mercury derivative crystal was R3, with unit cell dimensions a=b=143.994 Å, c=90.273 Å, α=β=90°, γ=120°. Single-wavelength (1.1 Å) anomalous dispersion of the mercury derivative was used for the calculation of anomalous difference Patterson. Fourteen sites were located by difference Patterson and difference Fourier maps (CNS, Brunger et al., Acta Crystallogr. D. Biol. Crystallogr., 54, pp. 905-921, (1998)). Phases calculated using these 14 sites were improved by combination of solvent flattening, histogram matching, phase extension and NCS averaging. Several cycles of model building (QUANTA2001, Accelrys) and phase combined refinement led to the initial model. The asymmetric unit contained two molecules. The model was extended by many cycles of rebuilding and refinement. The final model includes 14 mercury atoms and 133 water molecules positioned by ARP/WARP-REFMAC [A. Perrakis et al., Nat. Struct. Bio., 6, pp. 458-463 (1999)], and residues 46 to 385 for molecule A and residues 47 to 378 for molecule B.

In the above models, disordered residues were not included in the model. Alanine or glycine residues were used in the model if the side chains of certain residues could not be located in the electron density.

### EXAMPLE 5

### Overall Structure of Unphosphorylated MAPKAPK2

The structure of MAPKAPK2 used in the discussions and figures below is limited to molecule B coordinates except as otherwise indicated.

The crystal structure of unphosphorylated MAPKAPK2, determined at 2.8 Å resolution, includes a kinase domain and a C-terminal regulatory domain. Specifically, the MAPKAPK2 structure (FIG. 2) has the standard two-lobe kinase architecture plus an extra C-terminal regulatory domain. Although there are two molecules in an asymmetric unit (denoted molecules A and B), the structure shows no evidence of a dimer nor does MAPKAPK2 form dimers in solution. The two molecules are essentially identical, except at the bottom of the C-lobe of the kinase domain (residues 260-290, FIG. 4A). Due to the poor electron density in this region for both molecules, certain residues in the MAPKAPK2 sequence were not included in the model. Ser272, which is in this region, is one of the three major regulatory phosphorylation sites [R. Ben-Levy et al., EMBO. J., 14, pp. 5920-5930 (1995)]. The activation loop (residues 217-235) including Thr222, a common regulatory phosphorylation site in most serine/threonine kinases, is disordered in the structure (dotted line in FIG. 2).

Kinase Domain

The kinase domain of MAPKAPK2 resembles other known structures of kinases such as cAPK. Two lobes of the unphosphorylated MAPKAPK2 kinase domain take a “closed” conformation, which is usually the active form of phosphorylated kinases (FIG. 4B). Compared to the cAPK structure, the N-lobe of the MAPKAPK2 kinase domain starts with a long strand instead of a long helix (αA). The N-terminal part of the glycine rich loop (nucleotide binding loop) flips up 1200 and moves ˜11 Å to form a short helix (αB, corresponding to β1 of cAPK). The αB of cAPK is replaced by a three-residue turn in the MAPKAPK2 structure. The helix αC to residues DFG of the activation loop superimpose very well with the active cAPK structure. Moreover, the helices of the C-lobe superimpose nicely with the corresponding region in cAPK except for residues 260-290, which are poorly ordered and have different conformations in the two molecules in the asymmetric unit.

All catalytically important residues in the MAPKAPK2 structure can be aligned with the active form cAPK (FIG. 5). These residues include Lys93 (corresponding to Lys47 of cAPK), which binds to the phosphate of ATP and is localized by a salt bridge with Glu104 (corresponding to Glu62 of cAPK), Arg185 (corresponding to Arg140 of cAPK,), Asp186 (corresponding to Asp141 of cAPK, a conserved residue in all kinases), Asp207 (corresponding to Asp159 of cAPK, which coordinates Mg⁺²). Asp366 of the C-terminal regulatory domain of MAPKAPK2 occupies the position of phosphothreonine pThr195 in cAPK. A salt bridge between Arg185 and phosphothreonine (or phosphoserine) in the activation loop is critical for promoting the correct conformation of Asp186, the catalytic base, and for stabilizing positively charged residues Arg185 and Lys212 in the active form [L. N. Johnson et al., Cell, 85, pp. 149-158 (1996)].

C-terminal Regulatory Domain

The C-terminal regulatory domain of MAPKAPK2 is around residues 328-400. Deletion of this domain results in a marked increase in catalytic activity either with or without pretreatment by MAP kinase [Y. L. Zu et al., J. Biol. Chem., 270, pp. 202-206 (1995)] [K. Engel et al., J. Biol. Chem., 270, pp. 27213-27221 (1995)]. The C-terminal regulatory domain of MAPKAPK2 has a different conformation compared with that of cyclic AMP-dependent kinase [cAPK, (D. R. Knighton et al., Science, 253, pp. 414-420 (1991)]; 1FMO in Protein Data Bank], Calcium/calmodulin-dependent protein kinase I [CaMKI, J. Goldberg et al., Cell, 84, pp. 875-887 (1996)], and twitchin kinase [S. H. Hu et al., J. Mol. Biol., 236, pp. 1259-1261 (1994)].

There are two phosphorylation sites in this domain, Thr334 and Thr338. Thr334 is a major regulatory phosphorylation site. Thr338 is an auto-phosphorylation site [R. Ben-Levy et al., EMBO. J., 14, pp. 5920-5930 (1995)]. Both Thr334 and Thr338 are located in a very acidic environment. Phosphorylation of the two residues would be expected to weaken or interrupt the binding of the C-terminal regulatory domain to the catalytic domain.

In the MAPKAPK2 structure, the N-terminal part of this regulatory domain including the first helix (aJ) and the three-residue turn (residues 328-345) occupy very similar positions to those of αR1 and adjacent residues of CaMKI (J. Goldberg et al., Cell, 84, pp. 875-887 (1996), FIG. 4C). Although CaMKI does not have any phosphorylation sites in this region, however, Thr286 of CaKMII (corresponding to Val306 of CaMKI) is auto-phosphorylated when the enzyme is activated. The auto-phosphorylation site in MAPKAPK2, Thr338, occupies the same position as Thr286 of CaKMII [A. R. Means et al., Mol. Cell. Biol., 11, pp. 3960-3971 (1991)].

Interaction between conserved residue Glu145 (corresponding to Glu127 of cAPK, Glu102 of CaMKI) and Lys353, which mimics the P-3 arginine of the cAPK substrate analog PKI (Lys18, corresponding to Lys300 of CaMKI) supports the assumption that the C-terminal regulatory segment occupies the substrate binding pocket and may act like a pseudo-substrate. This substrate binding pocket of MAPKAPK2 is shown in FIG. 8. Phosphorylation of MAPKAPK2 by p38 at threonine 334 disrupts the interaction between the kinase domain and the C-terminal regulatory domain thus making the NES available for nuclear receptor binding. The tail of the second helix in MAPKAPK2 overlaps with the activation loop of cAPK (FIG. 4B, FIG. 6). The position of the cAPK phosphorylation site pThr195 is replaced by Asp366 as indicated above (FIG. 5, FIG. 6). In the MAPKAPK2 structure, the conformation of the long C-terminal strand, which appears to adopt its conformation solely for crystal packing, is flexible in solution.

MAPKAPK2 and its activator p38, are both located predominantly in the nucleus before stimulation. After stimulation, the proteins quickly translocate to the cytoplasm together [R. Ben-Levy et al., Curr. Biol., 8, pp. 1049-1057 (1998); K. Engel et al., EMBO. J., 17, pp. 3363-3371 (1998)]. The C-terminal regulatory domain of MAPKAPK2 (also MAPKAPK3/3pk) contains both a functional nuclear localization signal and a functional nuclear export signal [R. Ben-Levy et al., Curr. Biol., 8, pp. 1049-1057 (1998); K. Engel et al., EMBO. J., 17, pp. 3363-3371 (1998); T. Tanoue et al., EMBO. J., 20, pp. 466-479 (2001)]. NLS (residues ³⁷³KKX (10) KRRKK³⁸⁹) (SEQ ID NO: 6) of MAPKAPK2 is required for its activation by p38 in the nucleus. The NES of MAPKAPK2 (residues ³⁴⁵DKERWEDVKEEM TSALATMRVDYE³⁶⁸) (SEQ ID NO: 7) is sufficient to trigger nuclear export, which can be inhibited by leptomycin B, an inhibitor of the interaction between crm1/exportin 1 and Rev-type leucine-rich NES. The structure of the MAPKAPK2 NES is very similar to the NES of p53 [J. M. Stommel et al., EMBO. J., 18, pp. 1660-1672 (1999)] and the NES of 14-3-3 proteins [K. Rittinger et al., Mol. Cell, 4, pp. 153-166 (1999)] (FIG. 7A). All of them have three hydrophobic residues (Leu, Ile or Met) pointing to one side of the helix and another hydrophobic residue (Leu or Val) pointing to the other side of the helix (FIG. 7A, FIG. 8). Certain well known leucine-rich NES sequences are aligned with that of MAPKAPK2 in FIG. 7B.

Recent work [T. Tanoue et al., EMBO. J., 20, pp. 466-479 (2001)]) shows two distinct negatively charged regions of p38, namely the CD domain (Asp313, Asp315, Asp316 of p38) and the ED domain (Glu160, Asp161 of p38), are involved in the docking interaction with MAPKAPK3/3pk. A basic region of MAPKAPK3³⁶⁴KRRKK³⁶⁸ (SEQ ID NO: 8) (corresponding to ³⁸⁵KRRKK³⁸⁹ (SEQ ID NO: 9) of MAPKAPK2), which overlaps with the NLS, has been identified as the direct docking site for p38. Both the CD/ED domain of p38 and the KRRKK (SEQ ID NO: 8) residues are required for efficient phosphorylation of MAPKAPK3 by p38. It is possible that another basic region of MAPKAPK3 (³⁵⁰KIKD³⁵³) (SEQ ID NO: 10) (³⁷¹KIKK³⁷⁴ (SEQ ID NO: 11) of MAPKAPK2) is also involved in the interaction with p38. The distance between the carbon alpha atoms of Glu161^{p38} and Asp316^{p38} (18.99 Å) is similar to the distance between carbon alpha atoms of Lys374^{MAPKAPK2} and Lys385^{MAPKAPK2} (16.90 Å, within molecule A).

### EXAMPLE 6

### The Use of MAPKAPK2 Coordinates for Inhibitor Design

The coordinates of FIG. 1 are used to design compounds, including inhibitory compounds that associate with MAPKAPK2 or homologues of MAPKAPK2. This process may be aided by using a computer comprising a machine-readable data storage medium encoded with a set of machine-executable instructions, wherein the recorded instructions are capable of producing a three-dimensional structure or displaying a three-dimensional graphical representation of MAPKAPK2 or a portion thereof. The three-dimensional structure or graphical representation is used according to the methods described herein to design compounds. Such compounds associate with the MAPKAPK2 at the ATP or substrate binding pocket.

### EXAMPLE 7

### MAPKAPK2 Activity Inhibition Assay

Compounds were screened for their ability to inhibit MAPKAPK2 kinase 2 (MAPKAPK2) using a standard coupled enzyme assay [Fox et al Protein Sci. 7, 2249 (1998)]. Reactions were carried out in 100 mM HEPES 7.5, 10 mM MgCl₂, 25 mM NaCl, 1 mM DTT and 1.5% DMSO. Final substrate concentrations in the assay were 30 μM ATP (Sigma chemicals, St. Louis, Mo.) and 100 μM peptide (KKVNRTLSVA (SEQ ID NO: 12), American Peptide, Sunnyvale, Calif.). Assays were carried out at 30° C. with 20 nM MAPKAPK2. Final concentrations of the components of the coupled enzyme system were 2.5 mM phosphoenolpyruvate, 300 μM NADH, 30 μg/ml pyruvate kinase and 10 μg/ml lactate dehydrogenase.

An assay stock buffer solution was prepared containing all of the reagents listed above, with the exception of ATP and the test compound of interest. 59 μl of the reaction was placed in a 96 well plate followed by the addition of 1 μl of 3 mM DMSO stock containing the test compound (final compound concentration 30 μM). The plates were preincubated at 30° C. for 5 minutes, then the reaction was initiated by the addition of 7 μl of ATP (final concentration 30 μM). Rates of reaction were obtained by following the change in absorbance at 340 nm using a Molecular Devices plate reader (Sunnyvale, Calif.) over a 5 minute read time at 30° C. Standard wells contained DMSO but no test compound. Test compounds showing >50% inhibition compared to standard wells were titrated. Then, the IC₅₀'s of the test compounds were determined using a similar protocol in 96 well plates.

### EXAMPLE 8

### Identification of Residues in the ATP-Binding Pocket

Amino acid residues in the ATP-binding pocket were identified by superimposing the crystal structures of MAPKAPK2 and protein kinases such as cAMP-dependent protein kinase complexed with adenosine [Narayana et al., Biochemistry, 36, pp. 4438-48 (1997)] in the program QUANTA. First, residues that are in direct contact with the adenosine in the cAMP-dependent protein kinase structure are identified. Then, based on the superposition of the crystal structures of MAPKAPK2 and cAMP-dependent protein kinase, corresponding residues in MAPKAPK2 were identified. These residues include Lys77, Val78, Gln80, Ala91, Leu92, Lys93, Glu104, His108, Val18, Ile136, Met138, Glu139, Cys140, Leu141, Gly144, Glu145, Asp186, Glu190, Asn191, Leu193, Thr206 and Asp207.

### EXAMPLE 9

### Identification of Residues in the Substrate Binding Pocket

The program QUANTA was used to determine which residues in the MAPKAPK2 substrate binding site can interact with a substrate. Using the substrate NES bound to the substrate binding site as a model, residues Gln151, Phe158, Glu160, Arg185, Lys188, Tyr240, Leu256 and Leu257 were found to directly interact with the substrate. Residues Glu145, Phe147, Arg161, Ser164, Asp186, Pro189, Glu190, Phe210, Cys244, Trp247, Ser248, Val251, Ile252, Gly259, Tyr260 and Pro261 are proximal to the substrate binding pocket and are surface residues of that pocket.

While we have described a number of embodiments of this invention, it is apparent that our basic constructions may be altered to provide other embodiments that utilize the products, processes and methods of this invention. Therefore, it will be appreciated that the scope of this invention is to be defined by the appended claims, rather than by the specific embodiments that have been presented by way of example.

TABLE 1
Summary of data collection

| | |
|---|---|
| Wavelength (Å) | 1.1 |
| Resolution (Å) | 32.6-2.8 |
| No. of Reflections | 386,514/17,189 |
| (total/unique) | |
| Completeness (%) | 100.00 |
| R_{sym} (%) | 10.6 |
| Space group | R3 |
| Molecules per | 2 |
| asymmetric unit | |
| Unit cell | a = b = 143.994 Å, c = 90.273 Å, |
| | α = β = 90°, γ = 120° |

R_{sym} = Σ|I − <I>|/Σ/, where I is the observed intensity, <I> is the average intensity of the multiple observations of symmetry-related reflections.

Phasing

| | |
|---|---|
| R ₐₙₒ (%) | 0.066 |
| R _{cullis} (%) | 0.605 |
| Figure of Merit | 0.397 |
| Phasing Power | 1.833 |
| Number of heavy atom sites | 14 |

R ₐₙₒ = Σ|<I+> − <I−>|/Σ|<I+> + <I−>|
Phasing Power = rms. (|F_{H}|/E)ₚ, where |F_{H}| is the heavy atom structure factor amplitude and E is the residual lack of closure error.

Structure refinement

| | |
|---|---|
| R_{cryst} | 0.233 |
| Free R factor | 0.245 |
| RMS deviations (bond/angle) | 0.014 Å/3.2° |

Free F factor was calculated for a randomly chosen 5% of reflections;
R_{cryst} was calculated for the remaining 95% of the reflections used for structure refinement.

1. A crystalline molecule or molecular complex comprising a binding pocket, wherein said binding pocket is defined by structure coordinates of a set of amino acid residues which are identical to MAPKAPK2 amino acid residues Lys77, Leu92, His108, Ile136, Glu139 and Cys140 according to FIG. 1, wherein the root mean square deviation of the backbone atoms between said set of amino acid residues of said molecule or molecular complex and said MAPKAPK2 amino acid residues is not greater than about 3 Å.

2. The crystalline molecule or molecular complex of claim 1, wherein said set of amino acid residues further comprise amino acid residues which are identical to MAPKAPK2 amino acid residues Gln80 and Leu141 according to FIG. 1, wherein the root mean square deviation of the backbone atoms between said set of amino acid residues of said molecule or molecular complex and said MAPKAPK2 amino acid residues is not greater than about 3 Å.

3. A crystalline molecule or molecular complex comprising a binding pocket, wherein said binding pocket is defined by structure coordinates of a set of amino acid residues which are identical to MAPKAPK2 amino acid residues Gln151, Phe158, Glu160, Arg185, Lys188, Tyr240, Leu256 and Leu257 according to FIG. 1, wherein the root mean square deviation of the backbone atoms between said set of amino acid residues of said molecule or molecular complex and said MAPKAPK2 amino acid residues is not greater than about 3 Å.

4. The crystalline molecule or molecular complex of claim 3, wherein said set of amino acid residues further comprise amino acid residues which are identical to MAPKAPK2 amino acid residues Glu145, Phe147, Arg1601, Ser164, Asp186, Pro189, Glu190, Phe210, Cys244, Trp247, Ser248, Val251, Ile252, Gly259, Tyr260, Pro261 according to FIG. 1, wherein the root mean square deviation of the backbone atoms between said set of amino acid residues of said molecule or molecular complex and said MAPKAPK2 amino acid residues is not greater than about 3 Å.

5. A crystalline molecule or molecular complex comprising a binding pocket, wherein said binding pocket is defined by structure coordinates of a set of amino acid residues comprising at least two amino acid residues which are identical to MAPKAPK2 amino acid residues Lys77, Val78, Gln80, Ala91, Leu92, Lys93, Glu104, His108, Val118, Ile136, Met138, Glu139, Cys140, Leu141, Gly144, Glu145, Phe147, Gln151, Phe158, Glu160, Arg161, Ser164, Arg185, Asp186, Lys188, Pro189, Glu190, Asn191, Leu193, Thr206, and Asp207, Phe210, Tyr240, Cys244, Trp247, Ser248, Val251, Ile252, Leu256, Leu257, Gly259, Tyr260 and Pro261 according to FIG. 1, wherein the root mean square deviation of the backbone atoms between said set of amino acid residues of said molecule or molecular complex and said MAPKAPK2 amino acid residues is not greater than about 0.2 Å.

6. A crystalline molecule or molecular complex comprising a binding pocket, wherein said binding pocket is defined by structure coordinates of a set of amino acid residues which are identical to MAPKAPK2 amino acid residues Met356, Leu360, Met363 and Val365 according to FIG. 1, wherein the root mean square deviation of the backbone atoms between said set of amino acid residues of said molecule or molecular complex and said MAPKAPK2 amino acid residues is not greater than about 3 Å.

7. The crystalline molecule or molecular complex of claim 6, wherein said set of amino acid residues further comprise amino acid residues which are identical to MAPKAPK2 amino acid residues Asp345, Lys346, Glu347, Arg348, Trp349, Glu350, Asp351, Val352, Lys353, Glu354, Glu355, Thr357, Ser358, Ala359, Ala361, Thr362, Arg364, Asp366, Tyr367 and Glu368 according to FIG. 1, wherein the root mean square deviation of the backbone atoms between said set of amino acid residues of said molecule or molecular complex and said MAPKAPK2 amino acid residues is not greater than about 3 Å.

8. A crystalline molecule or molecular complex comprising a domain defined by structure coordinates of a set of amino acid residues which are identical to MAPKAPK2 amino acids 47-320 according to FIG. 1, wherein the root mean square deviation of the backbone atoms between said set of amino acid residues of said molecule or molecular complex and said MAPKAPK2 amino acid residues is not greater than about 5 Å.

9. A crystalline molecule or molecular complex comprising a domain defined by structure coordinates of a set of amino acid residues which are identical to MAPKAPK2 amino acids 321-400 according to FIG. 1, wherein the root mean square deviation of the backbone atoms between said set of amino acid residues of said molecule or molecular complex and said MAPKAPK2 amino acid residues is not greater than about 5 Å.

10. A crystalline molecule or molecular complex comprising a protein defined by structure coordinates of a set of amino acid residues which are identical to MAPKAPK2 amino acids according to FIG. 1, wherein the root mean square deviation of the backbone atoms between said set of amino acid residues of said molecule or molecular complex and said MAPKAPK2 amino acid residues is not greater than about 5 Å.

11. The crystalline molecule or molecular complex according to any one of claims 1-10, wherein the molecule is a MAPKAPK2 protein or a MAPKAPK2 protein homologue.

12. A crystal comprising a MAPKAPK2 protein or homologue thereof.

13. The crystal of claim 12, wherein the MAPKAPK2 protein or homologue is unphosphorylated or phosphorylated.

14. The crystal according to claim 12, wherein said MAPKAPK2 protein is selected from the group consisting of full length MAPKAPK2 protein, MAPKAPK2 protein with amino acid residues 47-400, amino acid residues 47-385 and amino acid residues 47-374 according to SEQ ID NO:

15. A method of obtaining a crystal comprising MAPKAPK2 protein or homologue thereof, comprising the steps of:
(a) producing and purifying MAPKAPK2 protein or homologue thereof;
(b) mixing said MAPKAPK2 protein or homologue thereof with a crystallization solution to produce a crystallizable composition; and
(c) subjecting said crystallizable composition to conditions that promote crystallization.

16. The method according to claim 15, wherein step (b) further comprises adding a chemical entity to the MAPKAPK2 protein or homologue thereof.

17. A computer comprising:
(a) a machine-readable data storage medium, comprising a data storage material encoded with machine-readable data, wherein said data defines the binding pocket according to any one of claims 1-7, the domain according to any one of claims 8-9, or the protein according to claim 10;
(b) a working memory for storing instructions for processing said machine-readable data;
(c) a central processing unit coupled to said working memory and to said machine-readable data storage medium for processing said machine-readable data and means for generating three-dimensional structural information of said binding pocket, domain or protein; and
(d) output hardware coupled to said central processing unit for outputting three-dimensional structural information of said binding pocket, domain or protein, or information produced using said three-dimensional structural information of said binding pocket, domain or protein.

18. The computer according to claim 17, wherein said means for generating three-dimensional structural information is provided by means for generating a three-dimensional structure of said binding pocket, domain or protein.

19. The computer according to claim 17 or 18, wherein said output hardware is a display terminal, a printer, CD or DVD recorder, ZIP™ or JAZ™ drive, a disk drive, or other machine-readable data storage device.

20. A method for designing, selecting and/or optimizing a chemical entity that binds to the molecule or molecular complex according to any one of the claims 1-10 comprising the steps of:
(a) providing the structure coordinates of said molecule or molecular complex on a computer comprising the means for generating three-dimensional structural information from said structure coordinates; and
(b) designing, selecting and/or optimizing said chemical entity by performing a fitting operation between said compound and said three-dimensional structural information of said molecule or molecular complex.

21. A method for evaluating the ability of a chemical entity to associate with the molecule or molecular complex according to any one of claims 1-10 comprising the steps of:
(a) employing computational means to perform a fitting operation between the chemical entity and the molecule or molecular complex; and
(b) analyzing the results of said fitting operation to quantitate the association between the chemical entity and the molecule or molecular complex.

22. The method according to claim 21, further comprising generating a three-dimensional graphical representation of the molecule or molecular complex prior to step (a).

23. The method of claim 21, wherein the method is for evaluating the ability of a chemical entity to associate with the binding pocket of the molecule or molecular complex.

24. A method of using a computer for evaluating the ability of a chemical entity to associate with the molecule or molecular complex according to any one of claims 1-7, wherein said computer comprises a machine-readable data storage medium comprising a data storage material encoded with said structure coordinates defining said binding pocket and means for generating a three-dimensional graphical representation of the binding pocket, and wherein said method comprises the steps of:
(a) positioning said chemical entity within all or part of said binding pocket using a graphical three-dimensional representation of the structure of the chemical entity and the binding pocket;
(b) performing a fitting operation between said chemical entity and said binding pocket by employing computational means;
(c) analyzing the results of said fitting operation to quantitate the association between said chemical entity and all or part of the binding pocket; and
(d) outputting said quantitated association to suitable output hardware.

25. The method according to claim 24, further comprising the steps of:
(e) repeating steps (a) through (d) with another chemical entity; and
(f) selecting at least one of said plurality of chemical entities that associates with said all or part of said binding pocket based on said quantitated association of said chemical entity.

26. A method for identifying an agonist or antagonist of a molecule or molecular complex according to any one of claims 1-10 comprising the steps of:
(a) using a three-dimensional structure of the molecule or molecular complex to design or select a chemical entity;
(b) contacting the chemical entity with the molecule or the molecular complex;
(c) monitoring the catalytic activity of the molecule or molecular complex; and
(d) classifying the chemical entity as an agonist or antagonist based on the effect of the chemical entity on the catalytic activity of the molecule or molecular complex.

27. A method of utilizing molecular replacement to obtain a structural model of a molecule or a molecular complex of unknown structure, comprising the steps of:
(a) crystallizing said molecule or molecular complex;
(b) generating an X-ray diffraction pattern from said crystallized molecule or molecular complex;
(c) applying at least a portion of the structure coordinates set forth in FIG. 1 or in a homology model thereof to the X-ray diffraction pattern to generate a three-dimensional electron density map of at least a portion of the molecule or molecular complex whose structure is unknown; and
(d) generating a structural model of the molecule or molecular complex from the three-dimensional electron density map.

28. The method according to claim 27, wherein the method is performed using a computer.

29. The method according to claim 27, wherein the molecule is a MAPKAPK2 homologue.

30. The method according to claim 27, wherein the molecular complex is selected from the group consisting of a MAPKAPK2 protein complex and a MAPKAPK2 homologue complex.


SEQUENCE LISTING (15 sequences)

SEQ ID NO: 1 - protein, 400 aa, Homo sapiens
MLSNSQGQSPPVPFPAPAPPPQPPTPALPHPPAQPPPPPPQQFPQFHVKSGLQIKKNAIIDDYKVTSQVLGLGINGKVLQIFNKRTQEKFALKMLQDCPKARREVELHWRASQCPHIVRIVDVYENLYAGRKCLLIVMECLDGGELFSRIQDRGDQAFTEREASEIMKSIGEAIQYLHSINIAHRDVKPENLLYTSKRPNAILKLTDFGFAKETTSHNSLTTPCYTPYYVAPEVLGPEKYDKSCDMWSLGVIMYILLCGYPPFYSNHGLAISPGMKTRIRMGQYEFPNPEWSEVSEEVKMLIRNLLKTEPTQRMTITEFMNHPWIMQSTKVPQTPLHTSRVLKEDKERWEDVKEEMTSALATMRVDYEQIKIKKIEDASNPLLLKRRKKARALEAAALAH

SEQ ID NO: 2 - protein, 382 aa, Homo sapiens
MDGETAEEQGGPVPPPVAPGGPGLGGAPGGRREPKKYAVTDDYQLSKQVLGLGVNGKVLECFHRRTGQKCALKLLYDSPKARQEVDHHWQASGGPHIVCILDVYENMHHGKRCLLIIMECMEGGELFSRIQERGDQAFTEREAAEIMRDIGTAIQFLHSHNIAHRDVKPENLLYTSKEKDAVLKLTDFGFAKETTQNALQTPCYTPYYVAPEVLGPEKYDKSCDMWSLGVIMYILLCGFPPFYSNTGQAISPGMKRRIRLGQYGFPNPEWSEVSEDAKQLIRLLLKTDPTERLTITQFMNHPWINQSMVVPQTPLHTARVLQEDKDHWDEVKEEMTSALATMRVDYDQVKIKDLKTSNNRLLNKRRKKQAGSSSASQGCNNQ

SEQ ID NO: 3 - protein, 332 aa, Homo sapiens
MPGAVEGPRWKQAEDIRDIYDFRDVLGTGAFSEVILAEDKRTQKLVAIKCIAKKALEGKEGSMENEIAVLHKIKHPNIVALDDIYESGGHLYLIMQLVSGGELFDRIVEKGFYTERDASRLIFQVLDAVKYLHDLGIVHRDLKPENLLYYSLDEDSKIMISDFGLSKMEDPGSVLSTACGTPGYVAPEVLAQKPYSKAVDCWSIGVIAYILLCGYPPFYDENDAKLFEQILKAEYEFDSPYWDDISDSAKDFIRHLMEKDPEKRFTCEQALQHPWIAGDTALDKNIHQSVSEQIKKNFAKSKWKQAFNATAVVRHMRKLQLGHQPGGTGTDS

SEQ ID NO: 4 - protein, 350 aa, Homo sapiens
  MOD_RES (10): Variable amino acid
GNAAAAKKGXEQESVKEFLAKAKEDFLKKWENPAQNTAHLDQFERIKTLGTGSFGRVMLVKHMETGNHYAMKILDKQKVVKLKQIEHTLNEKRILQAVNFPFLVKLEFSFKDNSNLYMVMEYVPGGEMFSHLRRIGRFSEPHARFYAAQIVLTFEYLHSLDLIYRDLKPENLLIDQQGYIQVTDFGFAKRVKGRTWTLCGTPEYLAPEIILSKGYNKAVDWWALGVLIYEMAAGYPPFFADQPIQIYEKIVSGKVRFPSHFSSDLKDLLRNLLQVDLTKRFGNLKNGVNDIKNHKWFATTDWIAIYQRKVEAPFIPKFKGPGDTSNFDDYEEEEIRVXINEKCGKEFSEF

SEQ ID NO: 5 - protein, 6 aa, Artificial Sequence
  Description of Artificial Sequence 6X-His tag
HHHHHH

SEQ ID NO: 6 - protein, 17 aa, Homo sapiens
  MOD_RES (3)..(12): Variable amino acid
KKXXXXXXXXXXKRRKK

SEQ ID NO: 7 - protein, 24 aa, Homo sapiens
DKERWEDVKEEMTSALATMRVDYE

SEQ ID NO: 8 - protein, 5 aa, Homo sapiens
KRRKK

SEQ ID NO: 9 - protein, 5 aa, Homo sapiens
KRRKK

SEQ ID NO: 10 - protein, 4 aa, Homo sapiens
KIKD

SEQ ID NO: 11 - protein, 4 aa, Homo sapiens
KIKK

SEQ ID NO: 12 - protein, 10 aa, Artificial Sequence
  Description of Artificial Sequence Synthetic peptide
KKVNRTLSVA

SEQ ID NO: 13 - protein, 16 aa, Homo sapiens
SNELALKLAGLDINKT

SEQ ID NO: 14 - protein, 16 aa, Homo sapiens
RFEMFRELNEALELKD

SEQ ID NO: 15 - protein, 16 aa, Homo sapiens
PVPLQLPPLERLTLDC